SEQUENCE LISTING (7 sequences)

SEQ ID NO: 1 - protein, 17 aa, Artificial Sequence
  Synthetic construct
MKKISSVIAIALFGTIA

SEQ ID NO: 2 - protein, 17 aa, Artificial Sequence
  Synthetic construct
VIAIALFGTIATANAAD

SEQ ID NO: 3 - protein, 17 aa, Artificial Sequence
  Synthetic construct
HQFTTKVIGKDSRDFDI

SEQ ID NO: 4 - protein, 17 aa, Artificial Sequence
  Synthetic construct
VIGKDSRDFDISPKVNG

SEQ ID NO: 5 - protein, 17 aa, Artificial Sequence
  Synthetic construct
RDFDISPKVNGENLVGD

SEQ ID NO: 6 - DNA, 21 bp, Artificial Sequence
  Synthetic construct
agctggtaaagcctggggcct

SEQ ID NO: 7 - DNA, 23 bp, Artificial Sequence
  Synthetic construct
cggtgactgaggttccttgaccc

(19) United States
(12) Patent Application Publication
(10) Pub. No.: US 2018/0360953 A1
(43) Pub. Date: Dec. 20, 2018

(54) METHODS TO IMPROVE INDUCTION OF IGG AND IGA ANTIBODIES BY VACCINES

(71) Applicant: OHIO STATE INNOVATION FOUNDATION, Columbus, OH (US)

(72) Inventors: Prosper Boyaka, Columbus, OH (US); Estelle Cormet-Boyaka, Columbus, OH (US)

(21) Appl. No.: 15/944,039

(22) Filed: Apr. 3, 2018

Related U.S. Application Data
(63) Continuation-in-part of application No. 15/566,884, filed on Oct. 16, 2017.
(60) Provisional application No. 62/148,044, filed on Apr. 15, 2015.

(51) Int. Cl. A61K 39/39; A61K 39/02; A61K 39/07; A61K 9/00

(52) U.S. Cl. CPC A61K 39/39; A61K 39/0291; A61K 2039/545; A61K 9/0056; A61K 9/0019; A61K 39/07

(57) ABSTRACT

A vaccine composition is disclosed that contains a vaccine antigen and a neutrophil inhibitor in amounts effective to promote an IgA and/or an IgG response to the antigen in a subject. Also disclosed is a method for enhancing immune response to a vaccine antigen in a subject that involves co-administering to the subject the vaccine antigen and an adjuvant composition comprising a neutrophil inhibitor in an amount effective to promote an IgA and/or IgG response to the vaccine antigen in the subject.

### CROSS REFERENCE TO RELATED APPLICATIONS

This application is a continuation-in-part application of U.S. application Ser. No. 15/566,884 filed Oct. 16, 2017, which is a national phase application filed under 35 U.S.C. § 371 of PCT/US2016/027891 filed Apr. 15, 2016, which claims the benefit of U.S. Provisional Patent Application Ser. No. 62/148,044 filed Apr. 15, 2015, the disclosures of which are expressly incorporated herein by reference.

### STATEMENT REGARDING FEDERALLY SPONSORED RESEARCH

This invention was made with Government Support under Grants No. R01AI043197 and R01DK101323 awarded by the National Institutes of Health. The Government has certain rights in the invention.

### BACKGROUND

Most infectious agents enter the body through mucosal tissues. Unfortunately, very few vaccines induce immunoglobulin-A (IgA), the type of immunoglobulins (antibodies) capable of protecting mucosal sites. Induction of mucosal IgA capable of providing a first line of defense against bacterial and viral pathogens remains a major goal of vaccines given via mucosal routes.

Also, most current vaccines are injected vaccines (parenterally delivered). Many of these vaccines contain alum (aluminum salts) as adjuvant. Current issues with vaccines are that they often require multiple boosts to achieve high serum antibody titers and to induce high affinity antibodies needed for protection. In addition, they do not stimulate expression of mucosal homing receptors by effector T and B cells and fail to induce mucosal IgA.

What is needed are improved vaccines and adjuvant compositions that can rapidly induce high affinity or functionally protective antibodies in the bloodstream, induce expression of mucosal homing receptors by effector B and T cells to generate two layers of protections against infectious agents, and induce IgA and/or IgG production.

### SUMMARY

A subset of myeloid cells is shown herein to provide a signal(s) that limits the ability of B lymphocytes to produce IgA and/or IgG antibodies. Disclosed herein are compositions and methods to limit recruitment of these cells in the sites where antibody responses develop after immunization and/or block the function of these cells and thus, limiting their ability to interfere with production of IgA antibodies by B lymphocytes. The methods described herein are not specific to a single vaccine and are expected to improve induction of IgA and/or IgG by most vaccine formulations regardless of the route of delivery.

A vaccine composition is therefore disclosed that contains a vaccine antigen and a neutrophil inhibitor in amounts effective to promote an IgA response to the antigen in a subject.

Also disclosed is an adjuvant composition comprising a neutrophil inhibitor and an adjuvant for combination with a vaccine antigen. Also disclosed is a method for enhancing immune response to a vaccine antigen in a subject that involves co-administering to the subject the vaccine antigen and an adjuvant composition comprising a neutrophil inhibitor in an amount effective to promote an IgA response to the vaccine antigen in the subject.

In some embodiments, the disclosed compositions and methods can be used to make nearly any vaccine a good inducer of IgA antibodies and high affinity IgG. For example, in some cases, the disclosed compositions and methods can be used to enhance or induce an IgA response for any vaccine antigen that is known to promote an IgG response in a subject. Therefore, the vaccine antigen can be a bacterial or viral antigen. Other useful antigens include protozoa, fungi, helminth, and ectoparasites.

In some embodiments, the disclosed adjuvant composition induces antigen-specific IgA in both the serum and mucosal secretions. Currently only vaccines given via mucosal routes (oral or intranasal) have the potential to induce mucosal IgA (i.e., if they contain appropriate adjuvants). In some embodiments, the disclosed adjuvant can enhance the ability of the vaccine antigen to induce IgA response in the mucosa when they are administered to a mucosal surface, such as by oral or intranasal administration. However, in other embodiments, the disclosed adjuvant allows induction of mucosal IgA for vaccines administered by other routes, such as by intramuscular or subcutaneous injection, by topical application under the tongue (sublingual), on the skin (i.e., epicutaneous as a patch), or by intravaginal administration.

The disclosed adjuvant can be co-administered with the vaccine antigen, e.g., in the same composition or contemporaneously by the same route of administration. In other embodiments, the disclosed adjuvant is administered contemporaneously by an alternative route of administration, e.g., intranasally for an injected vaccine or by injection for an intranasal vaccine. In other embodiments, the vaccine antigen is administered prior to the adjuvant composition. In an alternative embodiment, the vaccine antigen is administered after the adjuvant composition.

In some embodiments, the neutrophil inhibitor of the disclosed adjuvant is any agent that inhibits neutrophil elastase, phosphodiesterase type 4, neutrophil extracellular trap (NET), or neutrophil recruitment.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIGS. 1A to 1D show cholera toxin and Bacillus anthracis edema toxin promote different profiles of myeloid cell subsets in sublingual tissues. Sublingual tissues were collected 3 h after sublingual administration of phosphate-buffered saline (PBS), cholera toxin (CT) (2 μg) or Bacillus anthracis edema toxin (EdTx) (15 μg). FIG. 1A shows flow cytometry analysis of total myeloid cells. Top: absolute number; Bottom: frequency of CD11 b⁺ cells. FIG. 1B shows gating strategy for identification of myeloid cell subsets. FIG. 1C shows detailed flow cytometry analysis of myeloid cell subsets. FIG. 1D shows radar plots to summarize the profile of myeloid cell subsets in sublingual tissues. Data are expressed as mean±s.d. (n=3). *p≤0.05 compared with PBS.

FIGS. 2A to 2C show expression of phospho-NF-κB, phospho-STAT3, and profile of myeloid cell subsets in sublingual tissues after application of edema toxin (EdTx) to wild-type mice or genetically modified mice lacking IKKβ signaling in myeloid cells. Sublingual tissues were collected at different time points after sublingual administration of phosphate-buffered saline (PBS) or EdTx (15 μg). FIG. 2A is a representative Western-blot picture (from three independent experiments) of β-actin and overall (cytoplasmic and nuclear) phospho-NF-κB p65 (pNF-κB p65), and pSTAT3 levels in sublingual tissues 1 and 2 h after administration of EdTx. FIGS. 2B and 2C show flow cytometry analysis of myeloid cells (FIG. 2B) and cell subsets (FIG. 2C) 3 and 6 h after sublingual application of EdTx. All data are expressed as mean±s.d. (n=3). *p≤0.05 compared with PBS, and ▾p≤0.05 compared with C57BL/6.

FIGS. 3A to 3C show expression of chemokine and leukotriene B4 receptors by myeloid cell subsets in sublingual tissues. Sublingual tissues were collected 3 h after sublingual application of phosphate-buffered saline (PBS) or edema toxin (EdTx) (15 μg). Expression of CCR2, CXCR2, and LTB4R2 by myeloid cell subsets was analyzed by flow cytometry. FIG. 3A shows gating strategy for identification of chemoattractant receptors on myeloid cell subsets. FIG. 3B shows percentage of receptor-positive cells. FIG. 3C are pie diagrams of relative expression of individual receptors were generated using the formula: Relative number=(% positive cells for a receptor×1/sum of % positive cells for all receptors)×100. Data are expressed as mean±s.d. (n=4). *p≤0.05 receptor expression compared with C57BL/6 mice and ▾p≤0.05 compared with other receptors in the same group.

FIGS. 4A to 4C show lack of IKKβ signaling in myeloid cells improves the adjuvant activity of edema toxin (EdTx) after sublingual immunization and promotes antigen-specific secretory immunoglobulin-A (SIgA) responses. Mice were immunized three times at weekly intervals by sublingual application of the Yersinia pestis antigen F1-V alone or F1-V plus EdTx as an adjuvant. Serum, vaginal washes, and fecal samples were collected 1 week (Day 21), and saliva samples were collected 2 weeks (Day 28) after the last immunization. F1-V-specific Ab responses were analyzed by enzyme-linked immunosorbent assay (ELISA). FIGS. 4A and 4B show F1-V-specific serum antibody responses (FIG. 4A) and F1-V-specific SIgA responses in mucosal secretions (FIG. 4B). The end-point titers were expressed as Log 2 GMTs. ±s.d. from C57BL/6 (n=5), and IKKβ^{ΔMye} mice (n=3−5). *P≤0.05 compared with C57BL/6 mice and ▾p≤0.05 compared with group immunized with F1-V alone. FIG. 4C shows F1 epitope-specific serum IgG responses in C57BL/6 (n=5) and IKKβ^{ΔMye} mice (n=5). Sera were diluted 1:50 (groups immunized with F1-V alone) or 1:500 (groups immunized with F1-V plus EdTx), and IgG responses against linear epitopes of the capsular F1 antigen were analyzed by epitope-specific ELISA. Results were expressed as mean OD_{405 nm}±s.d. *p≤0.05 compared with group immunized with F1-V alone.

FIG. 5 shows lack of IKKβ in myeloid cells broadens antigen-specific T-helper cytokine responses to sublingual immunization with edema toxin as an adjuvant. Spleen cells were collected three weeks after the last immunization and cultured for 5 days in the presence of recombinant F1-V (5 μg m1⁻¹). The numbers of CD4⁺ T cells expressing Th1, Th2, and Th17 cytokines were analyzed by flow cytometry. Data are expressed as mean±s.d. from C57BL/6 (n=4) and IKKβ^{ΔMye} mice (n=4). *p≤0.05 compared with C57BL/6, and ▾p≤0.05 compared with mice immunized with F1-V alone.

FIGS. 6A to 6D show inverse correlation between the number of neutrophils and immunoglobulin-A (IgA) responses to sublingual immunization. Cervical lymph nodes were collected at 6 h after sublingual administration of edema toxin (EdTx) (15 μg). FIG. 6A shows flow cytometry analysis of myeloid cell subsets. FIG. 6B shows CLN cells further cultured for 3 days in the presence of lipopolysaccharide (5 μg ml⁻¹) and Ab-secreting cells analyzed by ELISPOT. FIG. 6C shows linear regression models to correlate the frequency of neutrophils and number of Ig isotype-secreting cells. Data are expressed as mean±s.d. (n=4). * p≤0.05 compared with C57BL/6. In FIG. 6D, wild-type C57BL/6 mice treated by IP administration of the neutrophil Ly6G-specific 1A8 monoclonal Ab (1A8¹ C57BL/6 mice). Two days later, control C57BL/6 and IKKβ^{ΔMye} mice were immunized three times at weekly intervals by sublingual application of F1-V plus EdTx. F1-V-specific IgA Ab responses in serum and fecal samples were analyzed by enzyme-linked immunosorbent assay (ELISA) and endpoint titers were expressed as Log₂ GMTs.±s.d. *p≤0.05 compared with C57BL/6 in each day (n=5).

FIGS. 7A to 7D show neutrophils suppress the production of immunoglobulin A (IgA) by B cells. In FIG. 7A, CD11b⁻ spleen cells from C57BL/6 mice were co-cultured with autologous CD11b⁺ cells from C57BL/6 mice or heterologous CD11b⁺ IKKβ^{ΔMye} cells in the presence of lipopolysaccharide (LPS; 5 μg ml⁻¹) with or without edema toxin (EdTx; 2 μg ml⁻¹). The frequencies of expression of IgA⁺ among B220⁺ IL-17RA^{low} and B220⁺ IL-17RA^{high} subpopulations were analyzed by flow cytometry after 5 days of co-culture with autologous or heterologous CD11b⁺ cells. Data are expressed as mean±s.d. (n=4). *p≤0.05 compared with C57BL/6 CD11b⁺ cells. In FIGS. 7B and 7C, CD19⁺ splenocytes from C57BL/6 mice were incubated overnight with 100 ng ml⁻¹ of LPS, washed extensively and then co-cultured with autologous neutrophils from C57BL/6 mice or heterologous neutrophils from IKKβ^{ΔMye} mice without additional stimuli. FIG. 7B shows IgM, IgG, and IgA levels in 5-day culture supernatants as determined by enzyme-linked immunosorbent assay. Data are expressed as mean±s.d. (n=4). *p≤0.05 compared with CD19⁺ B cells cultured alone. (c, d) Messenger RNA (mRNA) levels of IgA heavy chain determined by realtime reverse transcriptase-PCR after 24 h of co-culture. FIG. 7C shows individual mRNA levels in three independent experiments. FIG. 7D shows relative mRNA levels from co-culture of B cells with neutrophils as a percentage of mRNA levels in cultures of CD19⁺ B cells alone (n=3). *p≤0.05 compared with CD19⁺ B cells cultured alone.

FIGS. 8A to 8C show analysis of myeloid cell subsets recruited to sublingual tissues after sublingual application of EdTx. Sublingual tissues of C57BL/6 and IKKβ^{Mye} mice were collected 3 hours after sublingual administration of PBS or EdTx (15 μg). FIG. 8A shows gating strategy for identification of myeloid cell subsets, i.e., F4/80⁻ cells for neutrophils (I) and F4/80+ cells for the other myeloid subsets (II). FIG. 8B shows Geimsa staining and representative flow cytometry plots (SSC×FSC) of representative myeloid-lineage cells found in sublingual tissues of mice. FIG. 8C shows characteristic side scatter (SSC) of myeloid cell subsets. All data are expressed as mean±SD (n=6). * p≤0.05.

FIGS. 9A and 9B show expression of phospho-NF-κB and phospho-STAT3 in sublingual tissues after application of EdTx. Sublingual tissues of C57BL/6 or IKKβ^{ΔMye} mice were collected at different time points after sublingual administration of PBS or EdTx (15 μg). Expression of pSTAT3 and phosphoNF-κB p65 was analyzed by western blotting. FIG. 9A shows densitometry analysis of pNF-κB p65 and pSTAT3 protein in sublingual tissues 1 and 2 hours after administration of EdTx as normalized with β-actin. FIG. 9B shows variation of pSTAT3 and phosphoNF-κB p65 expression before (PBS) and 2 hours after administration of EdTx. Data are expressed as mean relative densitometry units±SD (n=3).

FIG. 10 shows profile of myeloid cell subsets in sublingual tissues. Sublingual tissues of C57BL/6 and IKKβ^{ΔMye} mice were collected at 3 and 6 hours after sublingual administration of PBS or EdTx (15 μg). Flow cytometry analysis of myeloid cell subsets determined by expression of CD11b, F4/80 and Gr-1. Results are expressed as mean percentage of three independent experiments and represented as radar plots to facilitate visual comparison between groups.

FIGS. 11A and 11B show kinetics of CCL2 (monocyte chemoattractant)-specific (FIG. 11A) and CXCL2 (neutrophils chemoattractant)-specific (FIG. 11B) mRNA responses in sublingual tissues of C57BL/6 or IKKβ^{ΔMye} mice exposed to EdTx in vitro. Single-cell suspensions of sublingual tissues from naïve mice were incubated with EdTx (2 μg/ml). At indicated time points, mRNA responses for CCL2 and CXCL2 were analyzed by real-time RT-PCR. Data represent mean±SD (n=3). * p≤0.05 compared to cultures without EdTx.

FIGS. 12A and 12B show lack of IKKβ^{ΔMye} signaling in myeloid cells enhances PA-specific responses after sublingual immunization with EdTx. Control C57BL/6 and IKKβ^{ΔMye} mice were immunized three times at weekly intervals by sublingual application of F1-V alone or F1-V plus EdTx as adjuvant. Serum samples were collected 1 week (Day 21) after the last immunization. FIG. 12A shows PA-specific IgG responses measured by ELISA. The end-point titers were expressed as Log₂ GMTs. ±SD from C57BL/6 (n=5), and IKKβ^{ΔMye} mice (n=5). * p≤0.05 compared with C57BL/6. FIG. 12B shows PA-specific neutralizing Ab titers determined by in vitro toxicity assay. Neutralizing Ab titers are expressed as Log₂ GMTs. ±SD from C57BL/6 (n=5), and IKKβ^{ΔMye} mice (n=5). *p≤0.05 compared with control C57BL/6 mice.

FIGS. 13A and 13B show Ag-specific serum (FIG. 13A) and fecal IgA (FIG. 13B) responses of IKKβ^{ΔMye} mice to sublingual vaccination. Control C57BL/6 and IKKβ^{ΔMye} mice were immunized three times at weekly intervals by sublingual application of F1-V alone plus cholera toxin (5 μg) as adjuvant. Serum and fecal extract samples were collected weekly, and antigen-specific IgA responses were measured by ELISA. The end-point titers are expressed as Log₂ GMTs. ±SD from C57BL/6 (n=5), and IKKβ^{ΔMye} mice (n=5). *p≤0.05 compared with C57BL/6. No response was detected in naïve mice.

FIGS. 14A and 14B show frequencies of myeloid cells in sublingual tissues and cervical lymph nodes after treatment with 1A8 mAb. Wild-type C57BL/6 mice were treated by ip administration of the neutrophil Ly6G-specific 1A8 monoclonal Ab or PBS (control). FIG. 14A shows the percentage of CD11b⁺F4/80⁻Gr-1^{high} (neutrophils) in the cervical lymph nodes was analyzed 1 (D1), 2 (D2) or 3 (D3) days later by flow cytometry. FIG. 14B shows the percentages of myeloid subsets in the sublingual tissues were analyzed by flow cytometry 3 hours after sublingual administration of PBS and EdTx (15 μg) in mice pretreated by ip injection of 1A8 (1A8=EdTx) or not (EdTx). Results are expressed as mean±SD (n=3).

FIGS. 15A to 15C show CD11b⁺IKKβ^{ΔMye} cells increase expression of surface IgA on B220⁺IL-17RA^{high} cells. CD11b⁻ spleen cells from C57BL/6 mice were co-cultured with autologous CD11b⁺ cells from C57BL/6 or heterologous CD11b⁺ IKKβ^{ΔMye} cells in the presence of LPS (5 μg/ml) with or without EdTx (2 μg/ml) for 5-day co-culture. FIG. 15A shows B cell (B220+) populations based on IL-17RA expression. FIG. 15B shows frequency of B220⁺IL-17RA^{low} and B220⁺IL-17RA^{high}. FIG. 15C shows APRIL, BAFF, and AID mRNA responses. Spleen cells from C57BL/6 mice were depleted of CD11b cells (CD11b⁻ spleen) and cultured for 24 hours at 37° C. with either autologous CD11b⁺ cells from C57BL/6 or heterologous CD11b⁺ cells from IKKβ^{ΔMye} mice (IKKβ^{ΔMye}CD11b⁺cells) in the presence or absence of EdTx (2 μg/ml). mRNA levels were determined by real time RT-PCR. Data are expressed as mean±SD and are representative of three independent experiments. *p≤0.05 compared with C57BL/6 CD11b⁺ cells, and ▾p≤0.05 compared with co-culture in the absence of EdTx.

FIGS. 16A to 16C show IKKβ^{ΔMye} CD11b⁺ cells and edema toxin regulate mRNA expression of cytokines and enzymes implicated in immunoglobulin class switch and Ab production. Spleen cells from C57BL/6 mice were depleted of CD11b cells (CD11b⁻ spleen) and cultured for 24 hours at 37° C. with either autologous CD11b⁺ cells from C57BL/6 or heterologous CD11b⁺ cells from IKKβ^{ΔMye} mice (IKKβ^{ΔMye} CD11b+ cells) in the presence or absence of EdTx (2 μg/ml). The mRNA levels of pro-inflammatory cytokines (FIG. 16A), anti-inflammatory cytokines (FIG. 16B) and Caspase 1 (FIG. 16C) were determined by real time RT-PCR. Data are expressed as mean±SD and are representative of three independent experiments. *p≤0.05 compared with C57BL/6 CD11b+ cells, and ▾p≤0.05 compared with co-culture in the absence of EdTx.

FIG. 17 shows OVA-specific serum IgG1 increases in mice treated with the 1A8 mAb before epicutaneous immunization. Neutrophils were depleted using the 1A8 mAb (administered to C57BL/6 mice i.p. on days −3, 4, and 11). The OVA antigen was given by epicutaneous immunization on intact skin (abdomen) using a volume of 100 μl. Mice were divided into three groups: 1) OVA only (1 mg) 2) OVA+cholera toxin (CT, 25 μg) and 3) OVA+B. anthracis edema toxin (EdTx: 50 μg of PA+50 μg of EF). Antibody responses in serum and mucosal secretion were examined at days 14, 21, and 28.

FIG. 18 shows OVA-specific serum IgG2c increases in mice treated with the 1A8 mAb. Neutrophils were depleted using the 1A8 mAb (administered to C57BL/6 mice i.p. on days −3, 4, and 11). The OVA antigen was given by epicutaneous immunization on intact skin (abdomen) using a volume of 100 μl. Mice were divided into three groups: 1) OVA only (1 mg) 2) OVA+cholera toxin (CT, 25 μg) and 3) OVA+B. anthracis edema toxin (EdTx: 50 μg of PA+50 μg of EF). Antibody responses in serum and mucosal secretion were examined at days 14, 21, and 28.

FIG. 19 shows OVA-specific serum IgA increases in mice treated with the 1A8 mAb. Neutrophils were depleted using the 1A8 mAb (administered to C57BL/6 mice i.p. on days −3, 4, and 11). The OVA antigen was given by epicutaneous immunization on intact skin (abdomen) using a volume of 100 μl. Mice were divided into three groups: 1) OVA only (1 mg) 2) OVA+cholera toxin (CT, 25 μg) and 3) OVA+B. anthracis edema toxin (EdTx: 50 μg of PA+50 μg of EF). Antibody responses in serum and mucosal secretion were examined at days 14, 21, and 28.

FIG. 20 shows induction of OVA-specific fecal IgA in mice treated with the −1A8 mAb. Neutrophils were depleted using the 1A8 mAb (administered to C57BL/6 mice i.p. on days −3, 4, and 11). The OVA antigen was given by epicutaneous immunization on intact skin (abdomen) using a volume of 100 μl Mice were divided into three groups: 1) OVA only (1 mg) 2) OVA +cholera toxin (CT, 25 μg) and 3) OVA+B. anthracis edema toxin (EdTx: 50 μg of PA+50 μg of EF). Antibody responses in serum and mucosal secretion were examined at days 14, 21, and 28.

FIG. 21 shows the neutrophil elastase inhibitor sivelestat enhances PA-specific serum IgG1 response induced by experimental intranasal vaccine containing CpG as adjuvant. C57BL/6 mice were given a nasal immunization of protective antigen of Bacillus anthracis (PA) (50 μg/dose given at a volume of 15 μl per nostril) three times at weekly intervals (days 0, 7, and 14). CpG adjuvant was administered at 5 μg/dose (CpG ODN 1826). Mice were divided into four treatment groups: (1) PA only (no Sivelestat) (2) PA+CpG (3) PA+neutrophil elastase inhibitor (Sivelestat) and (4) PA+CpG+neutrophil elastase inhibitor (Sivelestat). Antibody responses in serum and mucosal secretions were examined at day 21.

FIG. 22 shows the neutrophil elastase inhibitor sivelestat does not enhance PA-specific serum IgG2c response induced by experimental intranasal vaccine containing CpG as adjuvant. C57BL/6 mice were given a nasal immunization of protective antigen of Bacillus anthracis (PA) (50 μg/dose given at a volume of 15 μl per nostril) three times at weekly intervals (days 0, 7, and 14). CpG adjuvant was administered at 5 μg/dose (CpG ODN 1826). Mice were divided into four treatment groups: (1) PA only (no Sivelestat) (2) PA+CpG (3) PA+neutrophil elastase inhibitor (Sivelestat) and (4) PA+CpG+neutrophil elastase inhibitor (Sivelestat). Antibody responses in serum and mucosal secretions were examined at day 21.

FIG. 23 shows the neutrophil elastase inhibitor sivelestat enhances PA-specific serum IgA response induced by experimental intranasal vaccine containing CpG as adjuvant. C57BL/6 mice were given a nasal immunization of protective antigen of Bacillus anthracis (PA) (50 μg/dose given at a volume of 15 μl per nostril) three times at weekly intervals (days 0, 7, and 14). CpG adjuvant was administered at 5 μg/dose (CpG ODN 1826). Mice were divided into four treatment groups: (1) PA only (no Sivelestat) (2) PA+CpG (3) PA+neutrophil elastase inhibitor (Sivelestat) and (4) PA+CpG+neutrophil elastase inhibitor (Sivelestat). Antibody responses in serum and mucosal secretions were examined at days 14, 21, and 28.

FIG. 24 shows the neutrophil elastase inhibitor sivelestat enhances PA-specific salivary IgA response induced by experimental intranasal vaccine containing CpG as adjuvant. C57BL/6 mice were given a nasal immunization of protective antigen of Bacillus anthracis (PA) (50 μg/dose given at a volume of 15 μl per nostril) three times at weekly intervals (days 0, 7, and 14). CpG adjuvant was administered at 5 μg/dose (CpG ODN 1826). Mice were divided into four treatment groups: (1) PA only (no Sivelestat) (2) PA+CpG (3) PA+neutrophil elastase inhibitor (Sivelestat) and (4) PA+CpG+neutrophil elastase inhibitor (Sivelestat). Antibody responses in serum and mucosal secretions were examined at days 14, 21, and 28.

FIG. 25 is a schematic of genes and markers involved in the regulation of Ig class switching.

FIGS. 26A to 26D show inhibitors of neutrophil function differentially regulate immunoglobulin class switching and production of IgA responses. Spleen cells of mice cultured 24 hrs without or with neutrophil inhibitor (roflumilast (Phosphodiesterase-4 inhibitor), sivelestat (Neutrophil elastase inhibitor), sulfasalazine (IKKb inhibitor), or aspirin (COX inhibitor)). Gene expression was analyzed by qRT-PCR for BAFF, AID, TGFb, and IL-10. Mice were divided into 5 groups: (1) Control (Media) (2) Roflumilast (6.5 uM) (3) Sivelestat (46 uM) (4) Sulfasalazine (100 uM) and (5) Aspirin (100 uM). The different neutrophil inhibitors tested (roflumilast, sivelestat, sulfasalazine, and aspirin) differentially regulate genes involved in immunoglobulin class switching as shown for the genes B-cell activating factor (BAFF) (FIG. 26A), activation-induced cytidine deaminase (AID) (FIG. 26B), TGFβ (FIG. 26C), or IL-10 (FIG. 26D).

FIG. 27 shows neutrophil inhibitors enhance IgA antibody producing spleen cells cultured in the presence of the adjuvant cholera toxin B subunit (CTB). Spleen cells of nice mice were cultured for 4 days in the presence of cholera toxin B subunit (CT-B) in the presence or absence of a neutrophil inhibitor (roflumilast (6.5 μM), sivelestat (100 μM), sulfasalazine (100 μM), or aspirin (100 μM)). The frequency of IgA secreting cells was analyzed by ELISPOT.

FIGS. 28A to 28D show the neutrophil elastase inhibitor Sivelestat stimulates immunoglobulin class switching in vitro. Spleen cells were cultured for 5 days (A and D) or 2 days (B and C) in the presence of increasing doses of Sivelestat. (A) IgM, IgG and IgA secreted in culture supernatants were measured by ELISA. (B and C) Flow cytometry analysis. Cells were stained extracellularly with anti-CD19 and anti-CD138 and intracellularly with anti-IgG and anti-IgA Abs. (B) Gating strategy for analysis of IgG+CD138+ and IgA+CD138+ cells among CD19+ cells. (C) Percentage of CD138⁺IgG⁺ and CD138⁺IgA⁺ among CD19⁺ B cells. (D) Spleen cells were cultured for 5 days in the presence of LPS (0.05 μg/m1) and increasing doses of Sivelestat. The amounts of IgM, IgG and IgA secreted culture supernatants were measured by ELISA. Results are expressed as means±SD and are from at least 4 independent experiments. *P≤0.05, **P≤0.01, ***P≤0.05.

FIGS. 29A to 29D show the fluorinated elastase inhibitor Alvelestat stimulates immunoglobulin class switching and production of IgG and IgA in vitro. Spleen cells were cultured for 5 days (A and B) or 2 days (C and D) in the presence of increasing doses of Sivelestat. (A) IgM, IgG and IgA secreted in supernatants of cells cultured alone, or (B) IgA secreted in supernatants of cells cultured in the presence of LPS (0.05 μg/ml) were measured by ELISA. (C and D) Flow cytometry analysis. Cells were stained extracellularly with anti-CD19 and anti-CD138, and intracellularly with anti-IgG and anti-IgA Abs. (C) Gating strategy for analysis of IgG+CD138+ and IgA+CD138+ cells among CD19+ cells. (D) Percentage of CD138⁺IgG⁺ and CD138^{|} IgA^{|} among CD19^{|} B cells. (C) IgM, IgG and IgA secreted in supernatants of mesenteric lymph node cells after 5-days culture in the presence of Alvelestat were measured by ELISA. Results are expressed as means±SD and are from at least 4 independent experiments. *≤0.05, **P≤0.01, ***P≤0.05.

FIG. 30 shows the mechanisms underlying the stimulatory effect of neutrophil elastase inhibitor on IgG and IgA production. (A) Spleen cells were cultured for 24 hrs in the presence of Alvelestat (48 μμM) or Sivelestat (48 μM) and AID mRNA responses were measured by quantitative real-time RT-PCR. (B and C) Spleen cells were cultured for 24 hrs in the presence of Alvelestat or Sivelestat at the indicated doses. BAFF, APRIL (B) and IL-10 mRNA responses were measured by quantitative real-time RT-PCR. Results are expressed as means±SD and are from at least 3 independent experiments. *P≤0.05, **P≤0.01,***P≤0.05 compared to negative control.

FIGS. 31A to 31D show the neutrophil elastase inhibitor Sivelestat does not affect main immune cell subsets and but stimulates expression of surface IgG and surface IgA by cells of the B cell lineage. Spleen cells were cultured for 2 days in the presence of increasing doses of Sivelestat. The relative frequencies of immune cell subsets and surface expression of IgG or IgA were analyzed by flow cytometry after staining with combinations of the following Abs: anti-CD11b, anti-CD3e, anti-CD4, anti-CD8a, anti-CD19, anti-CD138, anti-IgG, and anti-IgA. (A) Frequency of myeloid cells. (B) Frequencies of T cell subsets. (C) Representative plots of IgG+CD138+ and IgA+CD138+ cells among CD19+ cells. (D) Percentage of CD138⁺IgG⁺ and CD138⁺IgA⁺ among CD19⁺ B cells. Results are expressed as means±SD (n=4 independent experiments). *P≤0.05, **P≤0.01, ***P≤0.05.

FIGS. 32A to 32D show the effects of the neutrophil elastase inhibitor Alvelestat on immune cell subsets and expression of surface IgG and surface IgA by cells of the B cell lineage. Spleen cells were cultured for 2 days in the presence of increasing doses of Alvelestat. The relative frequencies of immune cell subsets and surface expression of IgG or IgA were analyzed by flow cytometry after staining with combinations of the following Abs: anti-CD11b, anti-CD3e, anti-CD4, anti-CD8a, anti-CD19, anti-CD138, anti-IgG, and anti-IgA. (A) Frequency of myeloid cells. (B) Frequencies of T cell subsets. (C) Representative plots of IgG+CD138+ and IgA+CD138+ cells among CD19+ cells. (D) Percentage of CD138⁺IgG⁺ and CD138⁺IgA⁺ among CD19⁺ B cells. Results are expressed as means±SD (n=4 independent experiments). *P≤0.05, **P≤0.01, ***P≤0.05.

FIG. 33 shows that sublingual co-application of a NEI enhances antigen-specific serum IgG1 and IgG2b responses. Mice were immunized three times at weekly intervals by sublingual application of antigens (OVA plus PA) alone or antigens plus the NEI Alvelestat. To assess the effect of the NEI on the adjuvanticity of Bacillus anthraces edema toxin [EdTx, which results from combination of PA plus edema factor (EF)], mice were immunized with antigens plus EF or antigens plus EF plus NEI. Serum samples were collected weekly and OVA-specific Ab responses were analyzed by ELISA. (A) Kinetic of IgG1 Ab responses. (B and C) IgG2a/c, IgG2b and IgG3 Ab responses assessed: (B) one week (Day 21), or (C) two weeks (D28) after the last immunization. Data are expressed as mean Ab titers±SD (n=5 per group) and are from three independent experiments. *p≤0.05; **p≤0.01; ***p≤0.001.

FIG. 34 shows sublingual co-application of a NEI differentially affects antigen-specific serum IgE and IgA responses. Mice were immunized three times at weekly intervals by sublingual application of antigens (OVA plus PA) alone, antigens plus the NEI, antigens plus EF, or antigens plus EF plus NEI. Serum were collected weekly and OVA-specific Ab responses were analyzed by ELISA. (A) OVA-specific IgE responses on day 14. (B) OVA-specific IgA responses on days 21 and 28. Data are expressed as mean Ab titers±SD (n=5 per group) and are from three independent experiments. *p≤0.05; **p≤0.01; ***p≤0.001.

FIG. 35 shows sublingual co-application of a NEI promotes high affinity Abs with enhanced neutralizing activity. Mice were immunized three times at weekly intervals by sublingual application of antigens (OVA plus PA) alone, antigens plus the NEI, antigens plus EF, or antigens plus EF plus NEI. (A) Serum anti-PA neutralizing Ab responses. Serum samples were collected one week after the last immunization (Day 21), and PA-specific neutralizing Ab titers were determined by the in vitro macrophage toxicity assay. (B) PA-specific IgG1 and IgA Ab responses were determined by classical ELISA. (C) High affinity PA-specific IgG1 and IgA Ab responses. Antibody titers were assessed by a modified ELISA where low affinity Abs were removed by incubation with urea. Data are expressed as mean Ab titers±SD (n=5 per group) and are from three independent experiments. *p≤0.05; **p≤0.01; ***p≤0.001.

FIG. 36 shows sublingual co-application of a NEI promotes fecal IgA responses. Mice were immunized three times at weekly intervals by sublingual application of antigens (OVA plus PA) alone, antigens plus the NEI, antigens plus EF, or antigens plus EF plus NEI. Fecal pellets and vaginal washes were collected weekly and OVA-specific Ab responses were analyzed by ELISA. (B) OVA-specific IgA responses in fecal extracts on days 21 and 28. (B) OVA-specific IgA responses in vaginal washes on days 21 and 28. (C) OVA-specific IgG responses in vaginal washes on days 21 and 28. Data are expressed as mean Ab titers±SD (n=5 per group) and are from three independent experiments. *p≤0.05; **p≤0.01; ***p≤0.001.

FIG. 37 shows addition of NEI to a sublingual vaccine broaden the profile of CD4+ T helper cell responses. Mice were immunized three times at weekly intervals by sublingual application of antigens (OVA plus PA) alone, antigens plus the NEI, antigens plus EF, or antigens plus EF plus NEI. Spleen were collected two weeks after the last immunization and single cell suspensions were cultured for 5 days in the presence of OVA (1 mg/ml). Frequencies of (A) CD4+ Th1 cells; (B) CD4+ Th2 cells; (C) CD4+ Th17 and CD4+ Tfh cells were then analyzed by flow cytometry. Data are expressed as mean±SD (n=6 per group). *p≤0.05; **p≤0.01; ***p≤0.001.

FIG. 38 shows co-administration of NEI enhances the kinetic and magnitude of serum IgG in mice immunized parenterally immunized with alum as adjuvant. Mice were immunized three times at weekly intervals by i.p. injection of alum-adsorbed vaccine antigens (40 mg of OVA plus 20 mg of Bacillus anthracis PA) in the absence or presence of the NEI Alvelestat (100 or 200 μM). Blood was collected weekly and titers of OVA-specific serum Ab responses were determined by ELISA. Data are presented as mean antibody titers±one SD and represent one of at least four independent experiments with five mice per group. Data are expressed as mean±SD. *p≤0.05; **p≤0.01, **p≤0.001.

FIG. 39 shows co-administration of NEI or ELANE deficiency promotes high affinity Abs with enhanced neutralizing activity. Mice were immunized three times at weekly intervals by i.p. injection of alum-adsorbed vaccine antigens (40 mg of OVA plus 20 mg of Bacillus anthracis PA) alone or in the presence of the NEI Alvelestat (100 or 200 μM). ELANE deficient mice were immunized i.p. injection of alum-adsorbed vaccine antigens (40 mg of OVA plus 20 mg of Bacillus anthracis PA) alone. Serum samples were collected one week after the last immunization (Day 21). (A) High affinity PA-specific serum IgG1 and IgA Ab responses were determined by a modified ELISA where low affinity Abs were removed by incubation with urea (4 mM). (B) Serum anti-PA neutralizing Ab responses were determined by the Bacillus anthracis lethal toxin in vitro macrophage toxicity assay. Data are expressed as mean Ab titers±SD (n=5 per group) and are from three independent experiments. *p≤0.05; **p≤0.01; ***p≤0.001.

FIG. 40 shows co-administration of NEI promotes mucosal IgA responses in the gut of mice immunized parenterally with alum as adjuvant. Mice were immunized three times at weekly intervals by i.p. injection of alum-adsorbed vaccine antigens (40 mg of OVA plus 20 mg of Bacillus anthracis PA) in the absence or presence of the NEI Alvelestat. OVA-specific IgA and IgG were analyzed in fecal extracts (A) and vaginal washes (B) collected two weeks after the last immunization (day 28). Data are presented as mean antibody titers±one SD and represent one of at least four independent experiments with five mice per group. Data are expressed as mean±SD. *p≤0.05; **p≤0.01, **p≤0.001.

FIG. 41 shows co-administration of NEI promotes expression of gut homing receptors in mice immunized parenterally (i.p. injection) with alum as adjuvant. Mice were given alum-adsorbed vaccine antigens (40 mg of OVA plus 20 mg of Bacillus anthracis PA) alone or in the presence of the NEI Alvelestat (200 μM). Control mice were injected saline (PBS). (A) Spleens, (B) mesenteric lymph nodes, and (C) Peyer's patches were collected 24 hours later and the expression of mucosal homing receptors (i.e., CCR9 and a4b7) by B and T cells was analyzed by flow cytometry. Data are expressed as mean±SD (n=5). *P≤0.05; **p≤0.01, **p≤0.001.

FIG. 42 shows NEI promotes expression of gut homing receptors by B and T cells in vitro. Spleen (A) and mesenteric lymph node (B) cells of naïve mice were cultures for 48 hours in the absence or in the presence of the NEI Alvelestat (50 or 200 mM). The expression of mucosal homing receptors CCR9 and a4b7 by B lymphocytes, and T lymphocytes was analyzed by flow cytometry. Data are expressed as mean±SD (n=5). *P≤0.05; **p≤0.01, **p≤0.001.

FIG. 43 shows sublingual co-application of a NEI enhances antigen-specific serum IgG1 and IgG2b responses. Mice were immunized three times at weekly intervals by sublingual application of antigens (OVA plus PA) alone or antigens plus the NEI Alvelestat. To assess the effect of the NEI on the adjuvanticity of Bacillus anthraces edema toxin [EdTx, which results from combination of PA plus edema factor (EF)], mice were immunized with antigens plus EF or antigens plus EF plus NEI. Serum samples were collected weekly and OVA-specific Ab responses were analyzed by ELISA. (A) Kinetic of IgG1 Ab responses. (B) IgG2b responses two weeks (D28) after the last immunization. Data are expressed as mean Ab titers±SD (n=5 per group) and are from three independent experiments. *p≤0.05; **p≤0.01; ***p≤0.001.

FIG. 44 shows sublingual co-application of a NEI promotes high affinity Abs with enhanced neutralizing activity. Mice were immunized three times at weekly intervals by sublingual application of antigens (OVA plus PA) alone, antigens plus the NEI, antigens plus EF, or antigens plus EF plus NEI. (A) Serum anti-PA neutralizing Ab responses. Serum samples were collected one week after the last immunization (Day 21). (A) High affinity PA-specific IgG1 and IgA Ab responses. Antibody titers were assessed by a modified ELISA where low affinity Abs were removed by incubation with urea. (B) PA-specific neutralizing Ab titers were determined by the in vitro macrophage toxicity assay. Data are expressed as mean Ab titers±SD (n=5 per group) and are from three independent experiments. *p≤0.05; **p≤0.01; ***p≤0.001.

FIG. 45 shows sublingual co-application of a NEI suppresses antigen-specific serum IgE responses. Mice were immunized three times at weekly intervals by sublingual application of antigens (OVA plus PA) alone, antigens plus the NEI, antigens plus EF, or antigens plus EF plus NEI. Serum were collected weekly and OVA-specific IgE responses were analyzed by ELISA. Data are from samples collected on day 14, are expressed as mean Ab titers±SD (n=5 per group) and are from three independent experiments. *p≤0.05; **p≤0.01; ***p≤0.001.

### DETAILED DESCRIPTION

Definitions

The term “subject” or “patient” refers to any individual who is the target of administration or treatment. The subject can be a vertebrate, for example, a mammal. Thus, the subject can be a human or veterinary subject or patient. The term patient refers to a subject under the treatment of a clinician, e.g., physician. The term “subject” or “patient” includes humans and other mammals, including but not limited to murine (mice), rats, rabbit, horse, sheep, bovine, canines, cats, birds and any other warm-blooded animals classified as mammals. The term “subject” or “patient” also includes reptiles and amphibians.

The term “therapeutically effective” or “effective amount” refers to the fact that the amount of the composition used is of sufficient quantity to induce a protective immune response.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “inhibit” refers to a decrease in an activity, response, condition, disease, or other biological parameter. This can include but is not limited to the complete ablation of the activity, response, condition, or disease. This may also include a partial reduction, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The term “adjuvant” refers to a substance that enhances, augments or potentiates the host's immune response to a vaccine antigen.

The term “vaccine” refers to an organism or material that contains an antigen in an innocuous form. The vaccine may be recombinant or non-recombinant. When inoculated into a non-immune host, the vaccine will provoke active immunity to the antigen in the organism or material, but will not cause disease. Vaccines may take the form, for example, of a toxoid, which is defined as a toxin that has been detoxified but that still retains its major immunogenic determinants; killed organism, such as typhoid, cholera and poliomyelitis; or attenuated organisms that are the live, but non-virulent, forms of pathogens; or an antigen from such organism.

The term “antigen” or “immunogen” refers to a substance that has the ability to induce a specific immune response.

Vaccines

Neutrophil Inhibitor

In some embodiments, the neutrophil inhibitor of the disclosed vaccine adjuvant is any agent that inhibits neutrophil elastase. For example, in some embodiments, the neutrophil elastase inhibitor comprises sivelestat (Elaspol®, LY544349, ONO 5046, CAS # 127373-66-4).

In some embodiments, the neutrophil elastase inhibitor comprises a compound described in U.S. Pat. No. 8,063,073, which is incorporated by reference for the teaching of neutrophil elastase inhibitors. For example, in some embodiments, the neutrophil elastase inhibitor comprises alvelestat (AZD9668, CAS#848141-11-7).

In some embodiments, the neutrophil elastase inhibitor comprises the protein elafin (peptidase inhibitor 3, skin-derived antileukoprotease (SKALP)), or a functional fragment and/or variant thereof capable of inhibiting neutrophil elastase.

In some embodiments, the neutrophil elastase inhibitor comprises a protein of the serpin family, or a functional fragment and/or variant thereof capable of inhibiting neutrophil elastase. Examples of serpin family members are known in the art (See, for example, Law, et al. An overview of the serpin superfamily. Genome Biol. 2006; 7(5): 216).

In some embodiments, the neutrophil inhibitor is any agent that inhibits phosphodiesterase type 4 (PDE4). Examples of PDE4 inhibitors include Apremilast (Otezla®), Cilomilast (Ariflo®, SB-207,499), Diazepam (Valium®), Ibudilast (AV-411, MN-166), Luteolin, Mesembrenone, Piclamilast (RP 73401), Roflumilast (Daxas®, Daliresp®), and Rolipram.

In some embodiments, the neutrophil inhibitor is any agent that inhibits neutrophil extracellular trap (NET), such as sulfasalazine (Azulfidine®, Salazopyrin®, Sulazine®) or thalidomide (Immunoprin®, Talidex®, Talizer®, Thalomid®). Extracellular trap formation (ETosis) is a recently discovered form of cell death distinct from necrosis or apoptosis where a lattice of DNA strands is extruded from innate immune cells. Sulfasalazine and thalidomide have been shown to significantly inhibit ET formation in neutrophils and mast cells in a dose-dependent fashion.

In some embodiments, the neutrophil inhibitor is any agent that inhibits neutrophil recruitment. For example, the neutrophil inhibitor can be a CXCR1/2 antagonist, such as reparixin.

In some embodiments, the neutrophil inhibitor is any agent that inhibits IL-1R. For example, the IL-1R inhibitor can be anakinra (Kineret®). Anakinra blocks the biologic activity of naturally occurring IL-1, including inflammation and cartilage degradation associated with rheumatoid arthritis, by competitively inhibiting the binding of IL-1 to the Interleukin-1 type receptor, which is expressed in many tissues and organs.

In some embodiments, the neutrophil inhibitor is a non-steroidal anti-inflammatory drug (NSAID). NSAIDS include Salicylates, such as Aspirin (acetylsalicylic acid), Diflunisal (Dolobid®), Salicylic acid and other salicylates, and Salsalate (Disalcid); N-acetyl-para-aminophenol (APAP) derivatives, such as acetaminophen/paracetamol (Tylenol®); Propionic acid derivatives, such as Ibuprofen (Advil®, Motrin®, Nuprin®, and Medipren®), Dexibuprofen, Naproxen (Aleve®, Anaprox®, Antalgin®, Feminax Ultra®, Flanax®, Inza®, Midol® Extended Relief, Nalgesin®, Naposin®, Naprelan®, Naprogesic®, Naprosyn®, Naprosyn® suspension, EC-Naprosyn®, Narocin®, Proxen®, Synflex® and Xenobid®), Fenoprofen, Ketoprofen, Dexketoprofen, Flurbiprofen, Oxaprozin, and Loxoprofen; Acetic acid derivatives, such as Indomethacin (Indocin®), Tolmetin, Sulindac, Etodolac, Ketorolac, Diclofenac, Aceclofenac, and Nabumetone (Relafen®, Relifex®, Gambaran®); Enolic acid (Oxicam) derivatives, such as Piroxicam, Meloxicam, Tenoxicam, Droxicam, Lornoxicam, and Isoxicam; Anthranilic acid derivatives (Fenamates), such as Mefenamic acid, Meclofenamic acid, Flufenamic acid, and Tolfenamic acid; Selective COX-2 inhibitors (Coxibs), such as Celecoxib, Rofecoxib, Valdecoxib, Parecoxib, Lumiracoxib, Etoricoxib, and Firocoxib; and Sulfonanilides, such as Nimesulide.

In some embodiments, when an NSAID is used as neutrophil inhibitor, it is added to the organism or material that contains an antigen and co-administered by intranasal route, intramuscular or subcutaneous injection, or topical application under the tongue (sublingual) or on the skin (i.e., epicutaneous as a patch).

Antigens

In some embodiments, the disclosed compositions and methods can be used to enhance or induce an IgA response for any vaccine antigen that is known to promote an IgG response in a subject. Therefore, the vaccine antigen can be a bacterial or viral antigen.

Vaccines antigens for use in the disclosed compositions and methods can include one or more bacterial antigens from a particular bacteria. Bacteria for which vaccines can be formulated include, but are not limited to: Helicobacter pylori, Chlamydia pneumoniae, Chlamydia trachomatis, Ureaplasma urealyticum, Mycoplasma pneumoniae, Staphylococcus spp., Staphylococcus aureus, Streptococcus spp., Streptococcus pyogenes, Streptococcus pneumoniae, Streptococcus viridans, Enterococcus faecalis, Neisseria meningitidis, Neisseria gonorrhoeae, Bacillus anthraces, Salmonella spp., Salmonella typhi, Vibrio cholera, Pasteurella pestis, Pseudomonas aeruginosa, Campylobacter spp., Campylobacter jejuni, Clostridium spp., Clostridium difficile, Mycobacterium spp., Mycobacterium tuberculosis, Treponema spp., Borrelia spp., Borrelia burgdorferi, Leptospira spp., Hemophilus ducreyi, Corynebacterium diphtheria, Bordetella pertussis, Bordetella parapertussis, Bordetella bronchiseptica, hemophilus influenza, Escherichia coli, Shigella spp., Erlichia spp., Rickettsia spp., Yersinia pestis, Yersinia enterocolitica. Bacterial antigens can be native, recombinant or synthetic. Such bacterial antigens include, but are not limited to, selectins or lectins from bacteria that bind to carbohydrate determinants present on cell surfaces; and bacteria receptors for proteins, such as fibronectin, laminin, and collagens.

Vaccines antigens for use in the disclosed compositions and methods can include one or more antigens from a particular virus to form a vaccine. Viruses for which vaccines can be formulated include, but are not limited to: Influenza viruses, Parainfluenza viruses, Mumps virus, Adenoviruses, Respiratory syncytial virus, Epstein-Barr virus, Rhinoviruses, Polioviruses, Coxsackieviruses, rotaviruses, Echoviruses, Rubeola virus, Rubella virus, Varicella-zoster virus, Herpes viruses (human and animal), Herpes simplex virus, Parvoviruses (human and animal), Cytomegalovirus, Hepatitis viruses, Human papillomavirus, Alphaviruses, Flaviviruses, Bunyaviruses, Rabies virus, Arenaviruses, Filoviruses, HIV 1, HIV 2, HTLV-1, HTLV-II, FeLV, Bovine LV, FeIV, Canine distemper virus, Canine contagious hepatitis virus, Feline calicivirus, Feline rhinotracheitis virus, TGE virus (swine), and Foot and mouth disease virus. Viral antigens can be native, recombinant or synthetic. Such viral antigens include, but are not limited to, viral proteins that are responsible for attachment to cell surface receptors to initiate the infection process, such as (i) envelope glycoproteins of retroviruses (HIV, HTLV, FeLV and others) and herpes viruses, and (ii) the neuramidase of influenza viruses. Additionally, peptides derived from such viral proteins can be employed, either free, or associated non-covalently, or conjugated covalently to a suitable carrier.

Additional Adjuvants

In some embodiments, the disclosed adjuvant is used in combination with one or more other adjuvants. Adjuvants that have been used to enhance an immune response include aluminum compounds (all generally referred to as “alum”), oil-in-water emulsions (often containing other compounds), Freund's adjuvant (CFA or IFA), an oil-in-water emulsion containing dried, heat-killed Mycobacterium tuberculosis organisms), and pertussis adjuvant (a saline suspension of killed Bordetella pertussis organisms). These adjuvants generally are thought to have their mechanism of action by causing a depot of antigen and permitting a slow release of the antigen to the immune system, and by producing nonspecific inflammation thought to be responsible for their observed activity (Cox, J. C., et al., Vaccine 15:248-256 (1997)). Some saponins have been shown to have different types of immune stimulating activities, including adjuvant activity. These activities have been reviewed previously (Shibata, S., New Nat. Prod. Plant Pharmacol. Biol. Ther. Act., Proc. Int. Congr. 1st, 177-198 (1977); Price, K. R., et al. CRC Crit. Rev. Food Sci. Nutr. 26:27-135 (1987); Schopke, Th., & Hiller, K., Pharmazie 45:313-342 (1990); Lacaille-Dubois, M. A., et al., Phytomedicine 2:363-386 (1996).

Adjuvant could also consist of bacterial toxin derivatives either as subunit of toxins or detoxified derivatives of bacterial toxins, DNA with or without CpG oligodeoxynucleotides (ODN) (TLR9 ligands), or cyclic dinucleotides (STING ligands).

In some cases, the additional adjuvant is an aluminum salt (i.e., alum), TLR9 agonists (i.e., CpG oligodeoxynucleotides (ODN), TLR4 agonist (i.e., Monophosphoryl lipid A or MPL), agonist of other TLR molecules (i.e., TLR3 or TLR5), or a squalene-based oil-in-water emulsion (i.e., AS03, MF59).

Pharmaceutical Compositions

Disclosed is a pharmaceutical composition containing therapeutically effective amounts of one or more of the disclosed adjuvants and a pharmaceutically acceptable carrier. Pharmaceutical carriers suitable for administration of the adjuvants provided herein include any such carriers known to those skilled in the art to be suitable for the particular mode of administration. In addition, the adjuvants may be formulated as the sole active ingredient in the composition or may be combined with other active ingredients.

The compositions contain one or more adjuvants provided herein and may also contain one more vaccine antigens, as provide herein. The adjuvants are, in one embodiment, formulated into suitable pharmaceutical preparations such as solutions, suspensions, tablets, dispersible tablets, pills, capsules, powders, sustained release formulations or elixirs, for oral or sublingual administration or in sterile solutions or suspensions for parenteral administration, as well as transdermal patch preparation and dry powder inhalers. In one embodiment, the adjuvants described above are formulated into pharmaceutical compositions using techniques and procedures well known in the art (See, e.g., Ansel, Introduction to Pharmaceutical Dosage Forms, 4th Edition, 1985, 126).

In one embodiment, the compositions are formulated for single dosage administration. To formulate a composition, the weight fraction of compound is dissolved, suspended, dispersed or otherwise mixed in a selected carrier at an effective concentration such that the treated condition is relieved, or one or more symptoms are ameliorated.

The adjuvant is included in the pharmaceutically acceptable carrier in an amount sufficient to exert a therapeutically useful effect in the absence of undesirable side effects on the subject or patient treated. The therapeutically effective concentration may be determined empirically by testing the compounds in in vitro, ex vivo and in vivo systems, and then extrapolated therefrom for dosages for the subject or patient.

The concentration of adjuvants in the pharmaceutical composition will depend on absorption, inactivation and excretion rates of the adjuvants, the physicochemical characteristics of the adjuvants, the dosage schedule, and amount administered as well as other factors known to those of skill in the art.

Pharmaceutical dosage unit forms are prepared to provide from about 0.01 mg, 0.1 mg or 1 mg to about 500 mg, 1000 mg or 2000 mg, and in one embodiment from about 10 mg to about 500 mg of the active ingredient or a combination of essential ingredients per dosage unit form.

In instances in which the adjuvants exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN®, or dissolution in aqueous sodium bicarbonate.

Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, solubilizing agents, pH buffering agents and the like, for example, acetate, sodium citrate, cyclodextrin derivatives, sorbitan monolaurate, triethanolamine sodium acetate, triethanolamine oleate, and other such agents.

In one embodiment, the adjuvant is administered in a dose equivalent to parenteral administration of about 0.1 ng to about 100 g per kg of body weight, about 10 ng to about 50 g per kg of body weight, about 100 ng to about 1 g per kg of body weight, from about 1 μg to about 100 mg per kg of body weight, from about 1 μg to about 50 mg per kg of body weight, from about 1 mg to about 500 mg per kg of body weight; and from about 1 mg to about 50 mg per kg of body weight. Alternatively, the amount of adjuvant administered to achieve a therapeutic effective dose is about 0.1 ng, 1 ng, 10 ng, 100 ng, 1 μg, 10 μg, 100 μg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 30 mg, 40 mg, 50 mg, 60 mg, 70 mg, 80 mg, 90 mg, 100 mg, 500 mg per kg of body weight or greater.

Administration

The disclosed adjuvant compositions can be administered topically (patch), orally, parenterally, rectally, sublingually, by intravaginal administration, by inhalation or by direct gene transfer. The term “parenteral” includes subcutaneous injections, intravenous, intramuscular, intracisternal injection, or infusion techniques. Administration of the disclosed adjuvant can be done prior to, concurrent with or subsequent to the administration of the vaccine to the host. In any of the slow, controlled or sustained release formulations, the disclosed adjuvant will normally be administered concurrently with the vaccine, although the release characteristics of the disclosed adjuvant and the vaccine may differ in vivo. The disclosed adjuvant can be administered from 5 days prior to vaccine administration to about 30 days post vaccine administration. Such compositions will typically contain an immunogenicity-augmenting amount of the disclosed adjuvant, alone or in combination with an effective amount of any other active material. The dosages and desired drug concentrations contained in the compositions may vary depending upon many factors, including the intended use, patient's body weight and age, and route of administration. Preliminary doses can be determined according to animal tests, and the scaling of dosages for administration can be performed according to art-accepted practices without undue experimentation.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

### Neutrophils Negatively Regulate Induction of Mucosal IgA Responses After Sublingual Immunization.

Mucosal surfaces are constantly exposed to microorganisms and represent the main portal of entry of pathogens and toxins. Mucosal immunoglobulin-A (IgA) or secretory IgA (SIgA) neutralizes pathogenic microorganisms and toxins, interferes with bacterial or viral colonization of the epithelium, and participates in homeostasis of mucosal tissues (Macpherson, A. J., et al. Mucosal Immunol. 1, 11-22 (2008)). Ideally, vaccines capable of promoting both IgG in the bloodstream and SIgA in mucosal tissues would provide two layers of defense for optimal protection against infectious agents. Injected vaccines containing alum, the most widely used adjuvant, induce serum IgG responses, but unlike experimental mucosal adjuvants, fails to promote SIgA responses (Jackson, E. M., et al. PLoS One 7, e41529 (2012); Lo, D. D., et al. BMC Biotechnol. 12, 7 (2012)). Cholera toxin (CT) and the related heat labile toxin (LT) I of Escherichia coli are the most studied experimental adjuvants for induction of SIgA, however, their inherent toxicity precludes their use in oral or nasal vaccines.

Cytokines play a crucial role in shaping the profile of T-helper (Th) cytokine responses as well as the Ig isotype and subclass responses. Previous studies have shown that the mucosal adjuvant CT induces pro-inflammatory cytokine (i.e., interleukin-6 (IL-6) or IL-1β) secretion by antigen-presenting cells (i.e., macrophages and dendritic cells) (Cong, Y., et al. Eur. J. Immunol. 31, 64-71 (2001); Datta, S. K., et al. Proc. Natl Acad. Sci. USA 107, 10638-10643 (2010)). CT also induces transforming growth factor beta (TGF-β) and IL-10, two anti-inflammatory cytokines that play a central role in the induction of SIgA (Datta, S. K., et al. Proc. Natl Acad. Sci. USA 107, 10638-10643 (2010); Defrance, T., et al. J. Exp. Med. 175, 671-682 (1992); Kim, P. H., et al. J. Immunol. 160, 1198-1203 (1998)). Studies with live bacterial and viral vectors as well as immunization studies with Th1-inducing cytokines (i.e., IL-12 and IL-18) have now established that SIgA can also be induced in the context of Th1-biased responses. More recently, the ability of CT as an adjuvant to promote SIgA responses was shown to be impaired in mice lacking IL-17A, suggesting a role for IL-17A or related signaling in SIgA responses.6 In this regard, differentiation of Th17 cells requires IL-1β), IL-6, and TGF-β (Datta, S. K., et al. Proc. Natl Acad. Sci. USA 107, 10638-10643 (2010); Harrington, L. E., et al. Nat. Immunol. 6, 1123-1132 (2005)), which are cytokines that support IgA responses. Unlike Th1 and Th2 cytokines, which activate JAK-STAT signaling pathways, signaling through IL-17R activates Act1 for subsequent activation of the classical nuclear factor kB (NF-κB) signaling pathway (Gaffen, S. L., et al. Nat. Rev. Immunol. 9, 556-567 (2009)). Furthermore, IL-17A directly triggers Ig class switching to IgG2a and IgG3, but not to IgG1 (Mitsdoerffer, M., et al. Proc. Natl Acad. Sci. USA 107, 14292-14297 (2010)). To our knowledge, it is still unclear whether production of IgA is directly regulated by IL-17A/IL-17RA signaling in B cells. The NF-κB pathway plays an important role in inflammatory responses and a number of stimuli can lead to NF-κB translocation to the nucleus (Vallabhapurapu, S., et al. Annu. Rev. Immunol. 27, 693-733 (2009)). Previous studies have shown that the NF-κB pathway can mediate both pro- and anti-inflammatory effects (Greten, F. R., et al. Cell 130, 918-931(2007); Lawrence, T., et al. Nat. Med. 7, 1291-1297 (2001)) depending on the immune cells in which the IKKβ-NF-κB signaling occurs (Fong, C. H., et al. J. Exp. Med. 205, 1269-1276 (2008)) and stimuli to which they are exposed. A recent study showed a link between activation of the non-canonical NF-kB pathway in B cells and their ability to undergo immunoglobulin class switch for the production of IgA (Jin, J., et al. Nat. Immunol. 13, 1101-1109 (2012)). However, it remains unclear if IKKβ-dependent signaling in myeloid cells regulates IgA responses to mucosal vaccination.

Sublingual tissues have been used as a delivery site for bacterial and viral vaccines (Raghavan, S., et al. Infect. Immun. 78, 4251-4260 (2010); Song, J. H., et al. Proc. Natl Acad. Sci. USA 105, 1644-1649 (2008)), and cervical lymph nodes (CLNs) were identified as the primary site of antigen presentation after sublingual immunization (Song, J. H., et al. J. Immunol. 182, 6851-6860 (2009)). However, how innate immune cells in sublingual tissues and/or CLNs regulate antibody production remains unknown. Edema toxin (EdTx) is one of the exotoxins produced by the Gram-positive, spore-forming rod Bacillus anthracis (Moayeri, M., et al. Curr. Opin. Microbiol. 7, 19-24 (2004)). EdTx is composed of two subunits: a binding subunit and an enzymatic subunit. The binding subunit, or protective Ag (PA), allows the binding of these toxins to the anthrax toxin receptors that are expressed by most cells. The enzymatic subunit, or edema factor, is a calmodulin- and calcium-dependent adenylate cyclase that catalyzes the conversion of ATP to cyclic AMP (Moayeri, M., et al. Curr. Opin. Microbiol. 7, 19-24 (2004); Tang, W. J., et al. Mol. Aspects Med. 30, 423-430 (2009)). We previously showed that EdTx is a mucosal adjuvant that promotes mucosal and systemic immunity to intranasally co-administered vaccine antigens (Duverger, A., et al. J. Immunol. 185, 5943-5952 (2010); Duverger, A., et al. J. Immunol. 176, 1776-1783 (2006)). Here we addressed the contribution of monocytes/macrophages to mucosal SIgA responses after sublingual immunization. Using B. anthracis EdTx as a model of vaccine adjuvant to target anthrax toxin receptors, we show a previously unknown role of neutrophils as negative regulators of IgA responses. Thus, recruitment of neutrophils into sublingual tissues and CLNs shortly after sublingual immunization impaired the development of IgA responses. The negative role of neutrophils in IgA responses was confirmed in vivo by depletion of neutrophils before immunization with EdTx and in vitro by co-culture of B cells with neutrophils.

Methods

Mice. Control C57BL/6 mice were obtained from The Jackson Laboratory (Bar Harbor, Me.) or NCI-Frederick (Frederick, Md.) and acclimated to our facility for at least 2 weeks before being used. IKKβ^{ΔMye} mice were kindly provided by Dr Karin (University of California at San Diego) and were generated by crossing LysMCre mice, expressing Cre downstream of the lysozyme promoter in myeloid-lineage cells, with IKKβ^{f/f} mice harboring a loxP-flanked IKKβ gene (Greten, F. R., et al. Cell 130, 918-931 (2007); Lawrence, T., et al. Nat. Med. 7, 1291-1297 (2001)). Mice were bred in our facility, maintained in a pathogen-free environment and were used at 8-12 weeks of age. All experiments were performed in co-housed mice in accordance with both NIH and Institutional Animal Care and Use Committee guidelines.

Immunization and sample collection. Sublingual immunization was performed on mice anesthetized by intraperitoneal injection of ketamine and xylazine hydrochloride. The F1-V antigen and Bacillus anthracis protective antigen (PA) and edema factor (EF) were obtained from BEI Resources (Manassas, Va.). Mice were immunized three times, i.e., Day 0, 7, and 14 by administration of 30 μl of PBS containing 50 μg of F1-V antigen alone, or 50 μg of F1-V antigen plus 15 μg EdTx, i.e., 15 μg PA and 15 μg EF. To prevent accidental spill-over into the digestive tract, 15 μl of antigen plus adjuvant was administered twice at 6-hour intervals. Blood and external secretions (fecal extracts, vaginal washes, and saliva) were collected as previously described (Boyaka P N, et al. (2003) J Immunol 170(1):454-462). To identify the anatomic sites targeted by the adjuvant EdTx, mice were euthanized at 3, 6 or 12 hours after sublingual application of 15 μg EdTx. Sublingual tissues and CLNs were then collected and analyzed. To address the contribution of neutrophil to Ab isotype responses, mice were administered i.p. with 0.5 mg of the neutrophil Ly6G-specific 1A8 monoclonal Ab (BioXCell) 2 days before the sublingual immunization.

Immunoblot analysis of transcription factors. The expression of pNF-κB, pSTAT3, and β-actin was analyzed by immunoblot as previously described (Bonnegarde-Bernard A, et al. (2014) Mucosal Immunol 7(2):257-267) using anti-pNF-κB and pSTAT3 antibodies (Cell Signaling, Danvers, Mass.).

ELISA and ELISPOT assays for antigen-specific Ab responses and total Ig isotype levels. Antigen-specific Ab levels in plasma and external secretions were assessed as described previously on microtiter plates coated with 5 μg/ml of F1-V or PA (Duverger A, et al. (2010) J Immunol 185(10):5943-5952; Duverger A, et al. (2006) J Immunol 176(3):1776-1783). For analysis of B cell epitope responses, microtiter plates were coated with 50 μg/ml of overlapping peptides spamming the entire F1 molecule (BEI Resources). The frequency of antibody secreting cells (ASCs) was determined by ELISPOT assay with anti-IgM, IgG and IgA Abs (SBA, Birmingham, Ala.) (Boyaka P N, et al. (2003) J Immunol 170(1):454-462).

Macrophage toxicity assay to assess anti PA neutralizing Abs. The protective effects of PA-specific Abs were determined by analyzing survival of RAW 264.7 macrophages exposed to Bacillus anthraces lethal toxin in vitro as described previously (Duverger A, et al. (2010) J Immunol 185(10):5943-5952; Duverger A, et al. (2006) J Immunol 176(3):1776-1783).

In vitro cultures and flow cytometry analysis. To address the role of IKKβ in myeloid cells, myeloid cell-depleted CD11b⁻ splenocytes were co-cultured with autologous or heterologous CD11b⁺ cells at 7:1 ratios, i.e., 1.4×10⁵ to 0.2×10⁵ cells in the presence or absence of 5 μg/ml LPS (Sigma-Aldrich, St. Louis, Mo.) and 2 μg/ml EdTx. The CD11b⁻ and CD11b⁺ splenocytes were purified using CD11b MicroBeads with an autoMACS (Miltenyi Biotec, Auburn, Calif.).

For studies that addressed the direct effect of neutrophils on immunoglobulin production by B cells, CD19⁺ splenocytes from C57BL/6 mice were incubated overnight with 100 ng/ml of LPS, washed extensively and then co-cultured with autologous neutrophils from C57BL/6 mice or heterologous neutrophils from IKKβ^{ΔMye} mice without additional stimuli. The mRNA responses were analyzed 24 hours later, and culture supernatants were collected on day 5 for analysis of immunoglobulin isotypes (IgM, IgG, and IgA). For analysis of T helper cytokine responses, single-cell suspensions of spleen cells were incubated with 5 μg/ml F1-V at 37° C. for 5 days (Duverger A, et al. (2010) J Immunol 185(10):5943-5952; Duverger A, et al. (2006) J Immunol 176(3):1776-1783). Flow cytometry analysis was performed after extracellular and intracellular staining using the following fluorophore-conjugated monoclonal antibodies: B220, CD11b (Miltenyi Biotec, Auburn, Calif.), CD3e, CD4, CD8, IFN-γ, IL-4, IL-17A, I-A^{b} (MHC II), Gr-1, IgE, CCR2 and CXCR2 (BD Biosciences, San Jose, Calif.), F4/80 (AbD Serotec, Raleigh, N.C.), IL-17RA (eBiosciences, San Diego, Calif.), IgM, IgG and IgA (SBA, Birmingham, Ala.), LTB4R2 (Santa Cruz Biotechnology, Inc., Dallas, Tex.). Labeled cells were analyzed with an Accuri C6 flow cytometer (BD Biosciences, San Jose, Calif.)

Quantitative real-time RT-PCR. Extraction of total RNA, cDNA generation and RT-PCR were performed as previously described (Duverger A, et al. (2010) J Immunol 185(10):5943-5952). RT-PCR for the expression of IgA heavy chain transcripts was determined using the following forward and reverse primers set: 5′-AGC TGG TAA AGC CTG GGG CCT-3′ (SEQ ID NO:6) and 5′-CGG TGA CTG AGG TTC CTT GAC CC-3′ (SEQ ID NO:7) with cDNA as template. The level of mRNA expression in each target gene was calculated with the formula: 20−(mean of Ct_{target gene}−mean of Ct_{β-actin}).

Statistical analysis. Factorial ANOVA, followed by analysis of interaction effect and Duncan's multiple range test were used to compare values across categorical groups. Tests were considered significant at a probability of p≤0.05. Statistical analyses were performed with the Statistica 10.0 software package (StatSoft, Tulsa, Okla.).

Results

Toxin adjuvants differentially recruit myeloid-lineage cells into sublingual tissues

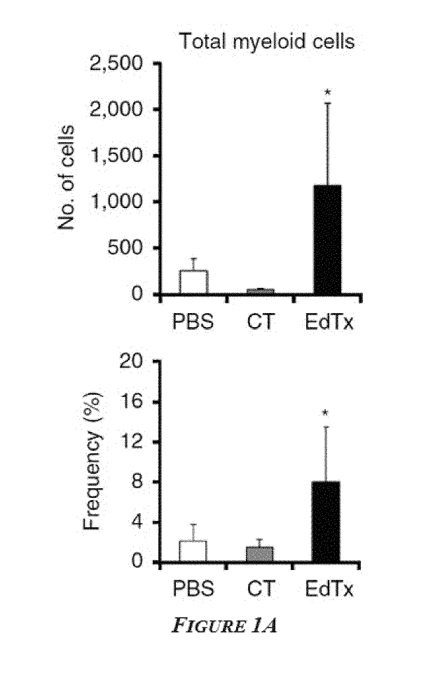

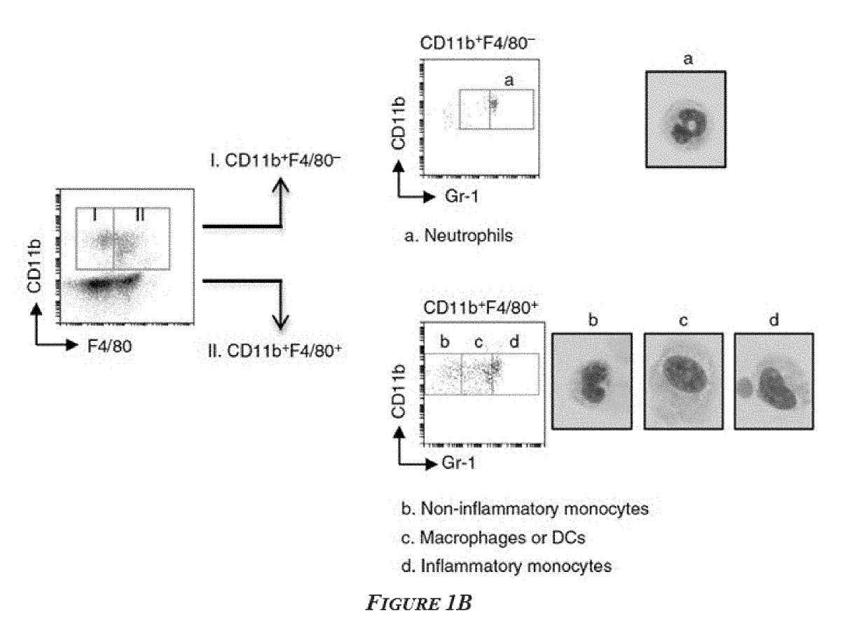

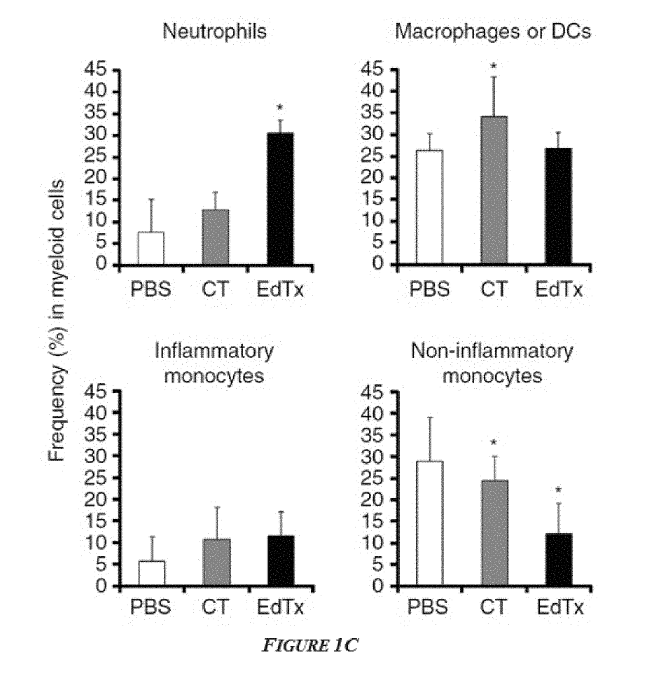

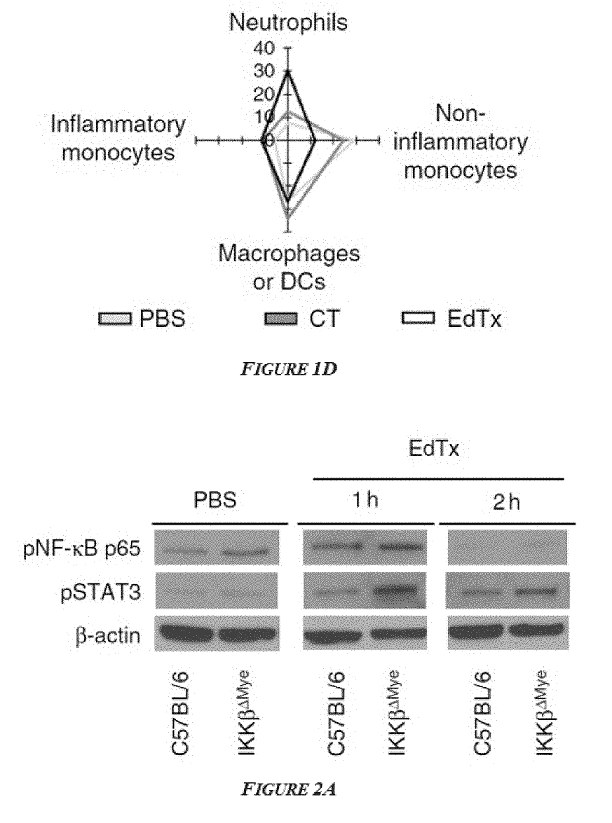

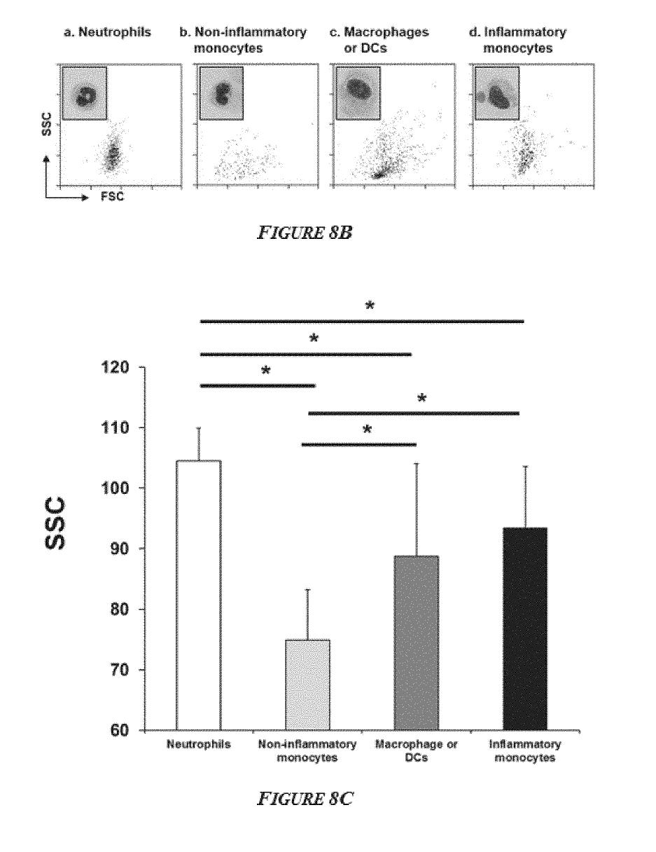

Both CT (Boyaka, P. N., et al. J. Immunol. 170, 454-462 (2003)) and EdTx (Duverger, A., et al. J. Immunol. 185, 5943-5952 (2010); Duverger, A., et al. J. Immunol. 176, 1776-1783 (2006)) are mucosal adjuvants that promote mucosal SIgA responses via the nasal route. CT can also promote IgA responses when used as an adjuvant for vaccines given by the epicutaneous route or topically on the sublingual mucosa (Song, J. H., et al. J. Immunol. 182, 6851-6860 (2009); Cuburu, N., et al. Vaccine 25, 8598-8610 (2007)). In contrast, EdTx was not effective at inducing IgA when used as an epicutaneous (Duverger et al., unpublished observation) or sublingual adjuvant. To elucidate the mechanism underlying the inability of EdTx to induce IgA by the sublingual route, innate cell subsets present in sublingual tissues after sublingual application of EdTx were first analyzed. The number of CD11b+ myeloid cells increased in the sublingual tissues of mice 3 h after application of EdTx, but not CT (FIG. 1A). Flow cytometric (Geissmann, F., et al. Immunity 19, 71-82 (2003)) and morphologic analysis of the myeloid cell subsets (FIGS. 8B to 8D) in sublingual tissues 3 h after application of EdTx showed a high frequency (32%) of CD11b⁺F4/80⁻Gr-1^{high} cells (neutrophils) and a lower frequency (12%) of CD11b⁺F4/80⁺Gr-1⁻cells (non-inflammatory monocytes). The frequencies of CD11b⁺F4/80⁺Gr-1^{low} cells (macrophages or DCs) and CD11b⁺F4/80⁺Gr-1^{high} cells (inflammatory monocytes) were not affected by EdTx. In contrast with EdTx, CT increased the frequency (34 vs. 26%) of macrophages/dendritic cell (FIG. 1C, 1D).

EdTx does not recruit neutrophils into sublingual tissues of mice lacking IKKβ in myeloid cells

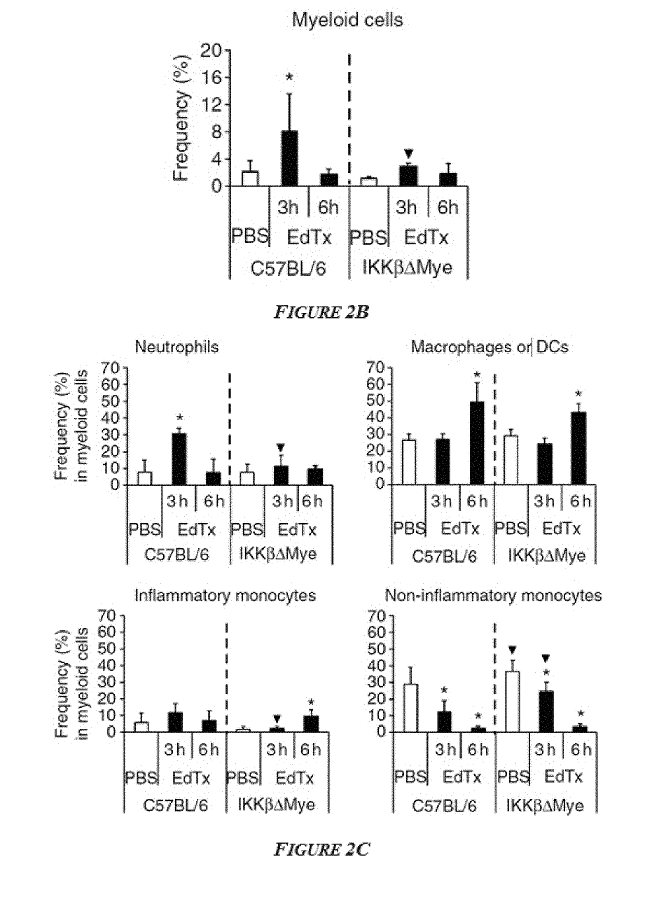

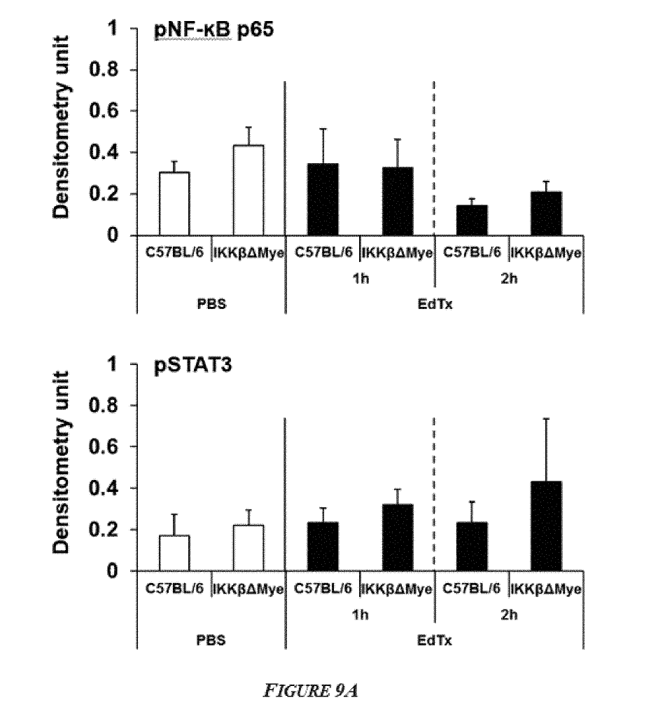

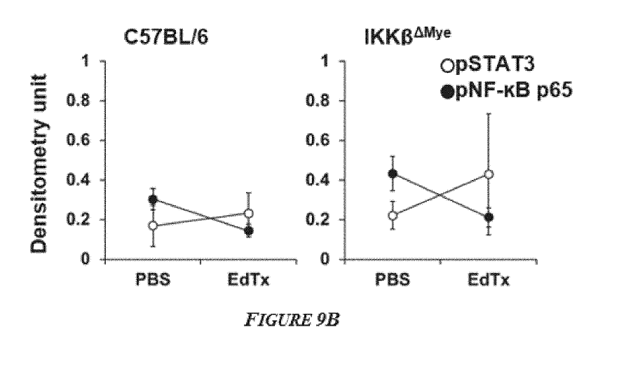

The adjuvant activities of CT and EdTx involve pro-inflammatory responses and acquisition of antigen-presenting cell functions by myeloid cells (Duverger, A., et al. J. Immunol. 185, 5943-5952 (2010); Duverger, A., et al. J. Immunol. 176, 1776-1783 (2006); Bromander, A. K., et al. Scand. J. Immunol. 37, 452-458 (1993); McGee, D. W., et al. J. Immunol. 151, 970-978 (1993)). The transcription factor NF-κB is a master regulator of cytokine responses and migration of innate cells (Grivennikov, S. I., et al. Cytokine Growth Factor Rev. 21, 11-19 (2010)). Activation of NF-κB in mouse epithelial cells lacking IKKβ and with impaired ability for nuclear translocation of phospoNF-κB p65, resulted in increased pSTAT3 responses in gut tissues (Bonnegarde-Bemard, A., et al. Mucosal Immunol. 7, 257-267 (2014)). Thus, how EdTx affects the expression of STAT3 in sublingual tissues of control and IKKβ^{ΔMye} mice, which lack IKKβ in myeloid cells, was examined. As depicted in FIGS. 2A and 9, pSTAT3 levels were low in tissues of mice that received saline and increased after application of EdTx. In contrast, pNF-κB levels were higher in tissues of phosphate-buffered saline- than in EdTx-treated mice. Although the transcription factors appeared to be regulated in the opposite direction after EdTx-treatment, the difference in their levels of expression failed to reach statistical significance during the time frame analyzed (FIGS. 2A and 9).

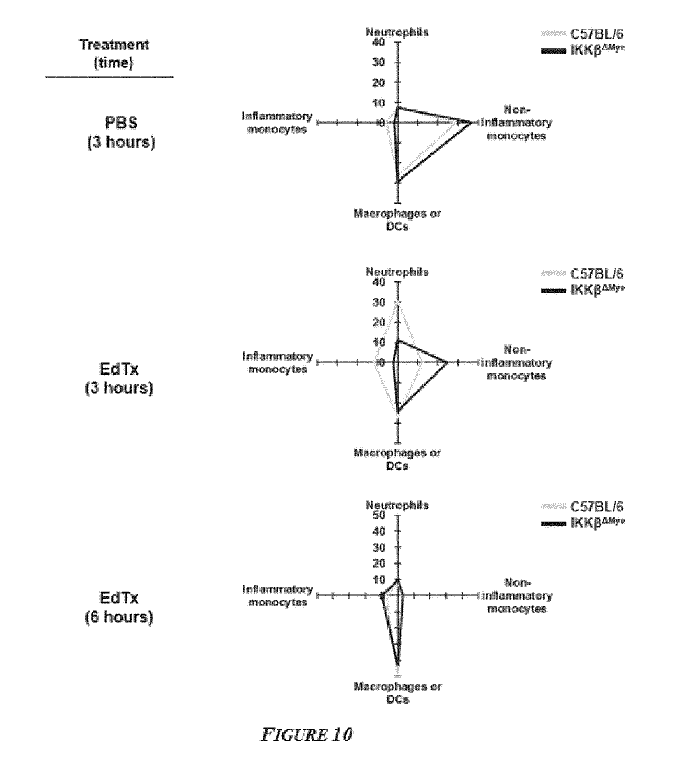

Myeloid cells in sublingual tissues were also analyzed at 3 and 6 h after application of EdTx (FIGS. 2B, 2C, and 10). Unlike control C57BL/6 mice, the IKKβ^{ΔMye} mice did not exhibit an important increase in the frequency of CD11b [symbol] cells in the sublingual tissues 3 h after application of EdTx (FIG. 2B). Control C57BL/6 and IKKβ^{ΔMye} mice exhibited a similar proportion of myeloid cell subsets before application of EdTx, except for non-inflammatory monocytes, which were higher in IKKβ^{ΔMye} than in control C57BL/6 mice (FIGS. 2C and 10). Three hours after application of EdTx, sublingual tissues of IKKβ^{ΔMye} mice showed significantly lower frequencies of neutrophils when compared with C57BL/6 (FIGS. 2C and 10).

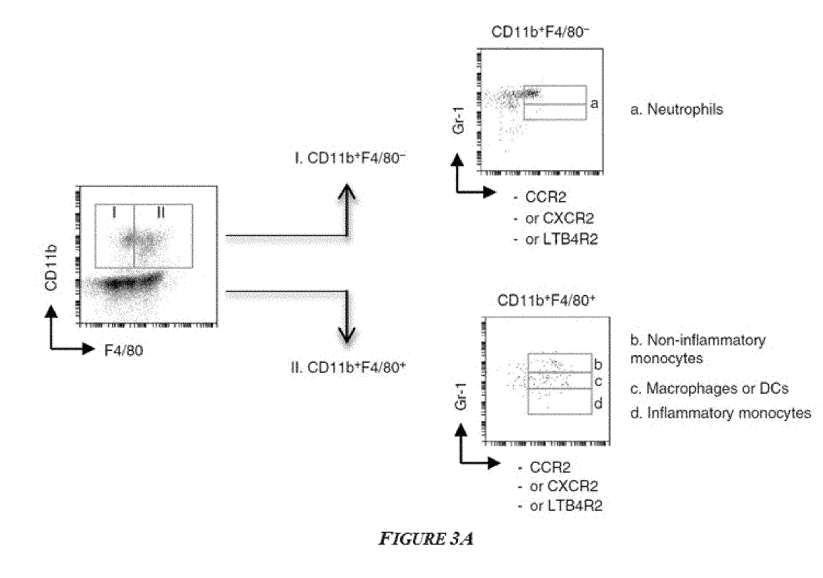

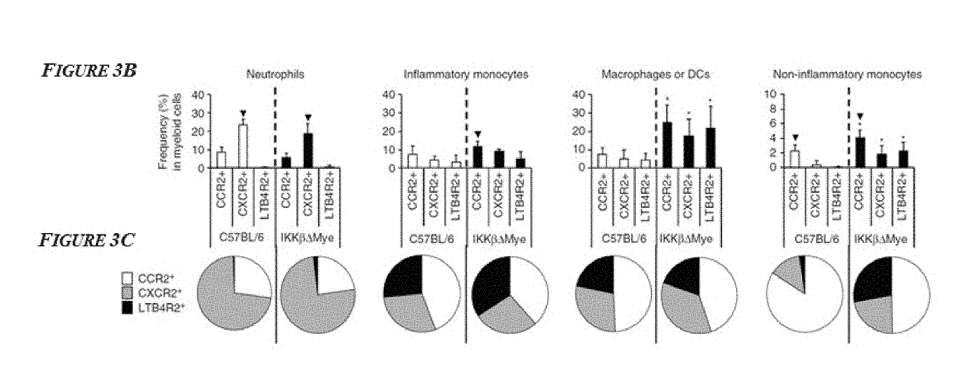

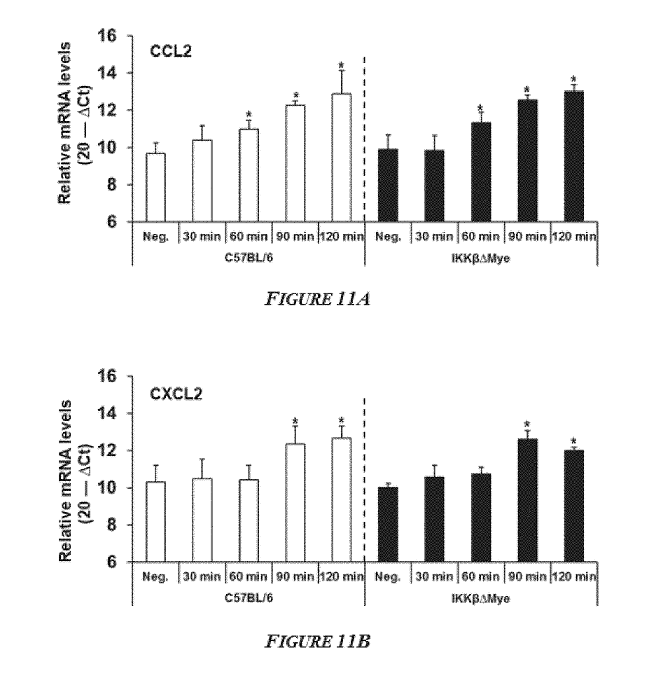

EdTx was reported to differentially affect the recruitment and cytokine secretion by some immune cells (Klezovich-Benard, M., et al. PLoS Pathog. 8, e1002481 (2012)). Therefore, the expression of CCL2 and CXCL2, two chemokines known to recruit inflammatory monocytes and neutrophils, respectively, was also examined. Sublingual tissue cells of control C57BL/6 and IKKβ^{ΔMye} mice had similar basal levels of CCL2 and CXCL2 messenger RNA (mRNA), and exhibited similar kinetics and magnitude of responses after exposure to EdTx (FIG. 11). The expression of CCR2 and CXCR2, the receptors of CCL2 and CXCL2 by myeloid cell subsets, was also examined in sublingual tissues 3 h after application of EdTx in vivo (FIG. 3). Since leukotriene B4 could mediate chemotaxis of macrophages and granulocytes (Grespan, R., et al. Arthritis Rheum. 58, 2030-2040 (2008); Chou, R. C., et al. Immunity 33, 266-278 (2010)), the expression of the leukotriene B4 receptor (LTB4R2) was also investigated. Neutrophils in sublingual tissues of C57BL/6 and IKKβ^{ΔMye} mice exhibited similar profiles of receptor expression (FIGS. 3B, 3C). On the other hand, macrophages/DCs and non-inflammatory monocytes collected in sublingual tissues of IKKβ^{ΔMye} mice exhibited higher frequencies of CCR2⁺, CXCR2⁺, and LTB4R2⁺ cells. Alone, these results cannot explain the higher number of neutrophils in the sublingual tissues of C57BL/6. The pie diagram (FIG. 3C), which summarizes the relative contribution of each receptor in myeloid cell subsets shows a broader profile of receptor expression in macrophages/DCs and non-inflammatory monocytes of IKKβ^{ΔMye} mice. Thus, these cells may have a competitive advantage for responding to chemoattractant signals via ligand binding to these receptors.

IKKβ Deficiency in Myeloid Cells Enhances the Adjuvant Activity of EdTx for Sublingual Immunization and Promotes Ag-Specific SIgA Responses

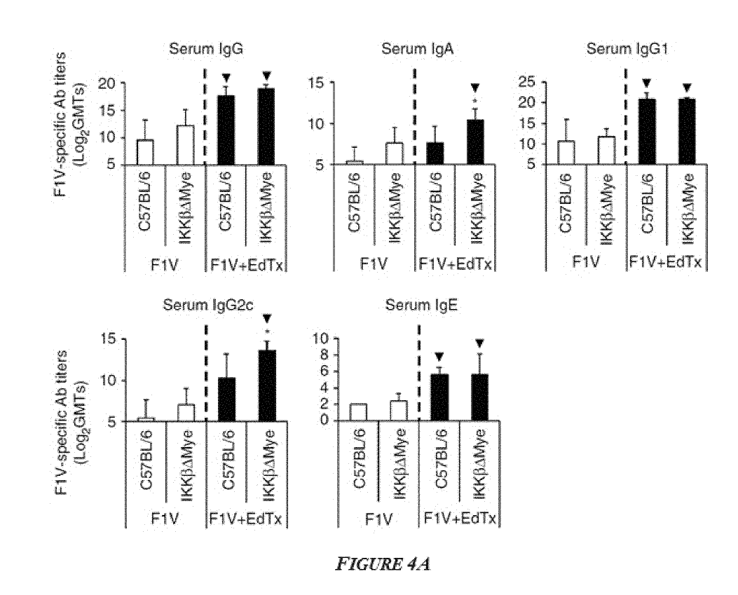

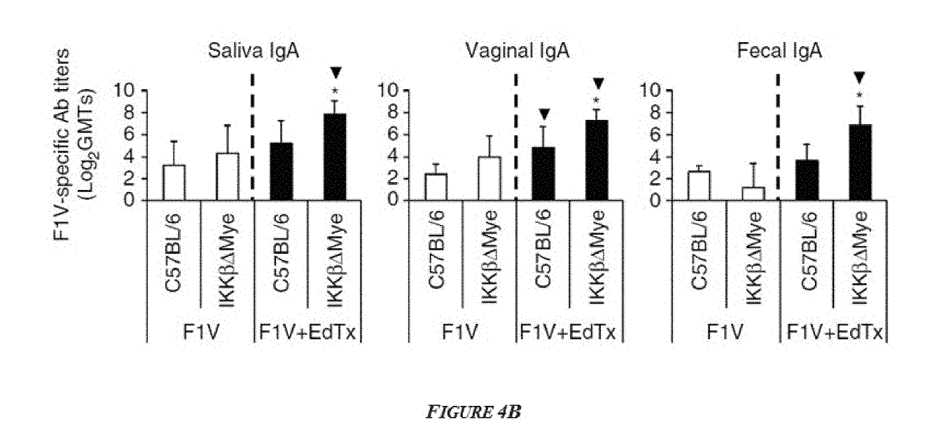

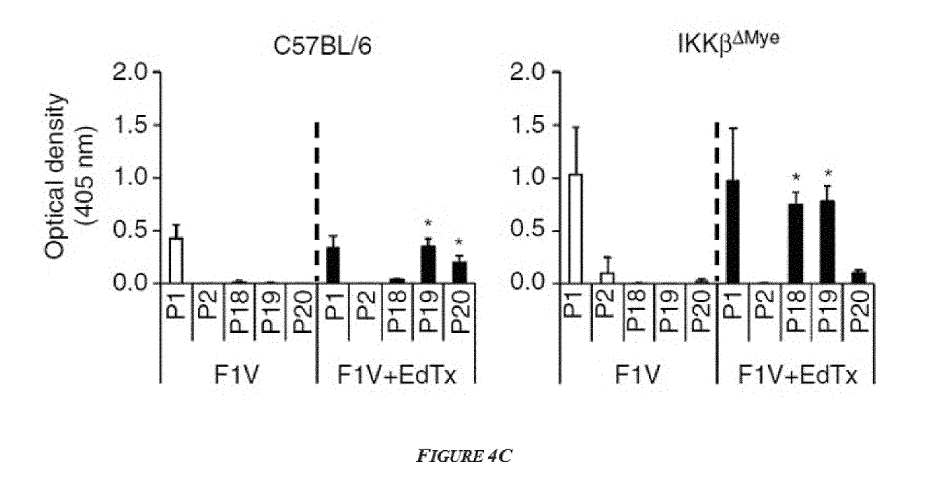

Experiments were conducted to determine whether the broader expression of chemokine receptors and LTB4R2s by macrophage/dendritic cell and non-inflammatory monocytes in sublingual tissues of IKKβ^{ΔMye} mice after application of EdTx could affect the profile of immune responses induced by this adjuvant. For this purpose, mice were immunized via the sublingual route with recombinant Yersinia pestis F1-V antigen and B. anthraces EdTx as adjuvant. Sublingual co-application of EdTx enhanced antigen (F1-V)-specific serum IgG responses (IgG and IgG1) and no difference was seen between control C57BL/6 and IKKβ^{ΔMye} mice (FIG. 4A). The similar levels of IgE responses were seen in control C57BL/6 and IKKβ^{ΔMye} mice, suggesting that Th2-dependent Abs were not affected in IKKβ^{ΔMye} mice. On the other hand, IKKβ^{ΔMye} mice exhibited enhanced IgG2c responses (FIG. 4A). Interestingly, unlike control C57BL/6 mice, IKKβ^{Mye} mice developed antigen-specific serum IgA responses, (FIG. 4A). The increase in serum IgA responses in IKKβ^{ΔMye} mice was associated with antigen-specific SIgA in the saliva, vaginal washes, and fecal extracts (FIG. 4B). Experiments were also conducted to determine whether the specificity and function of antibody (Ab) induced by EdTx as sublingual adjuvant were affected by the absence of IKKβ-dependent signaling in myeloid cells. After sublingual immunization with F1-V alone, control C57BL/6 and IKKβ^{ΔMye} mice developed IgG Abs, which were directed against the same peptide (i.e., P1-17 or P1) of the F1-capsular antigen (FIG. 4C and Table 1). EdTx as an adjuvant promoted Abs that reacted to two additional epitope peptides in control C57BL/6 and IKKβ^{ΔMye} mice. However, only one of the additional peptides (P19) was shared by Abs from control C57BL/6 and IKKβ^{ΔMye} mice (FIG. 4C and Table 1).

TABLE 1
Linear epitopes of Yersinia pestis F1-capsular antigen recognized
by antibodies after sublingual immunization

| Peptide | Residues | Sequence | |
|---|---|---|---|
| P1 | 1-17 | 5′-MKKISSVIAIALFGTIA-3′ | SEQ ID NO: 1 |
| P2 | 7-23 | 5′-VIAIALFGTIATANAAD-3′ | SEQ ID NO: 2 |
| P18 | 103-119 | 5′-HQFTTKVIGKDSRDFDI-3′ | SEQ ID NO: 3 |
| P19 | 109-125 | 5′-VIGKDSRDFDISPKVNG-3′ | SEQ ID NO: 4 |
| P20 | 115-131 | 5′-RDFDISPKVNGENLVGD-3′ | SEQ ID NO: 5 |

Abbreviation ELISA, enzyme-linked immunosorbent assay.
Mice were immunized three times at weekly intervals by sublingual application of F1-V alone or F1-V plus EdTx as an adjuvant. Serum samples were collected 1 week (Day 21) after the last immunization, and the B-cell epitope responses were evaluated by ELISA using overlapping peptides spamming the all F1 molecule (BEI Resources, Manassas, VA). Shown are the sequences and corresponding residues of peptides that reacted with sera of mice immunized with F1-V alone or F1-V plus EdTx as an adjuvant.

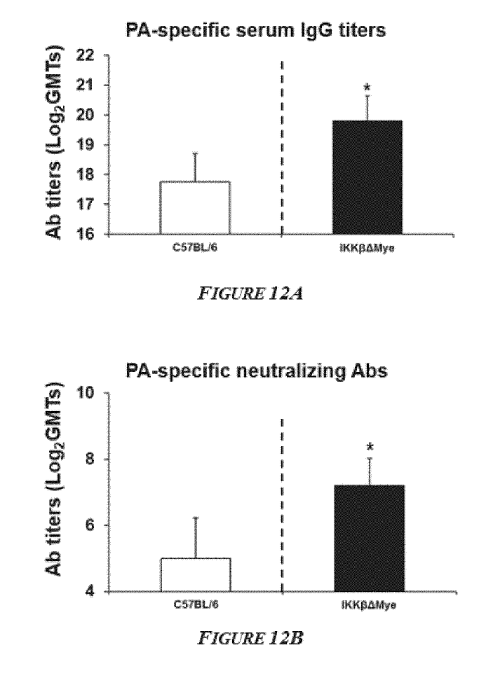

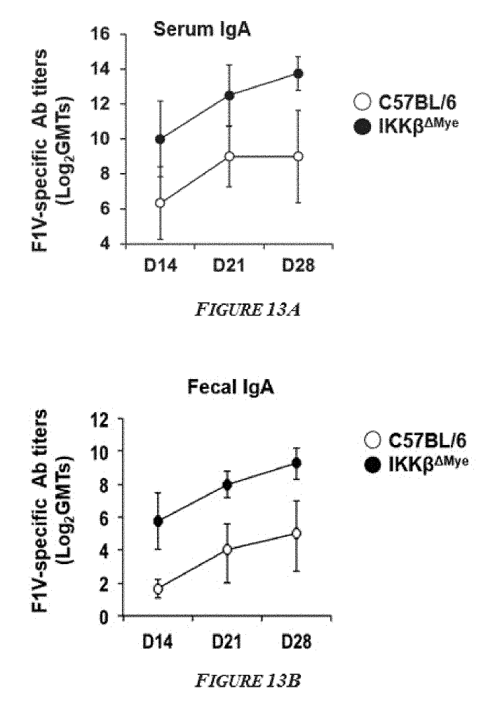

Experiments were also conducted to determine whether the enhanced IgA responses seen in IKKβ^{ΔMye} mice were restricted to FI-V as antigen and EdTx as adjuvant. Nasal immunization with EdTx is known to promote immunity against the EdTx binding subunit PA (Duverger, A., et al. J. Immunol. 185,5943-5952 (2010); Duverger, A., et al. J. Immunol. 176,1776-1783 (2006)). Sublingual immunization with EdTx also enhanced PA-specific serum IgG Ab titers in IKKβ^{ΔMye} mice (FIG. 12A) and this was consistent with the enhanced levels of PA-specific neutralizing Abs (FIG. 12B). In addition, serum and mucosal IgA responses induced by CT as a sublingual adjuvant were enhanced in IKKβ^{ΔMye} mice (FIG. 13).

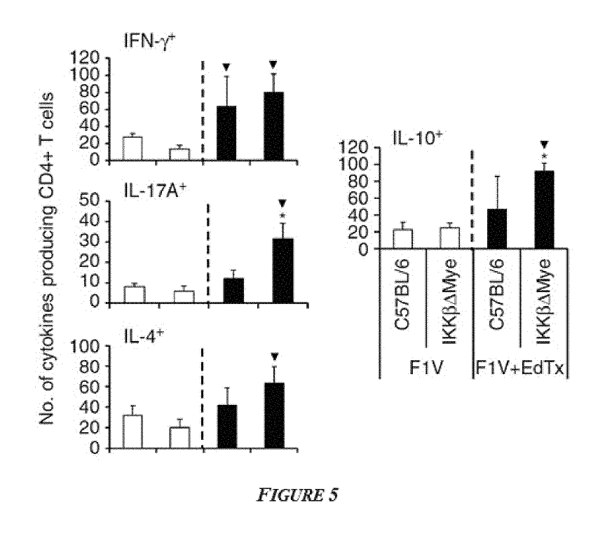

Finally, antigen (F1-V)-specific Th cytokine responses supported by EdTx as an adjuvant for sublingual vaccination was analyzed. In wild-type C57BL/6 mice, the sublingual adjuvant EdTx enhanced the frequency of antigen-specific IFN-γ producing Th cells in the spleen (FIG. 5). On the other hand, the IKKβ^{ΔMye} mice exhibited a broader profile of Th-cell responses with a significant increase of antigen-specific IFN-γ (Th1), IL-4 (Th2), IL-10⁺, and IL-17A⁺ producing Th cells (FIG. 5).

The Frequency of Neutrophils Inversely Correlates with Production of IgA in CLNs

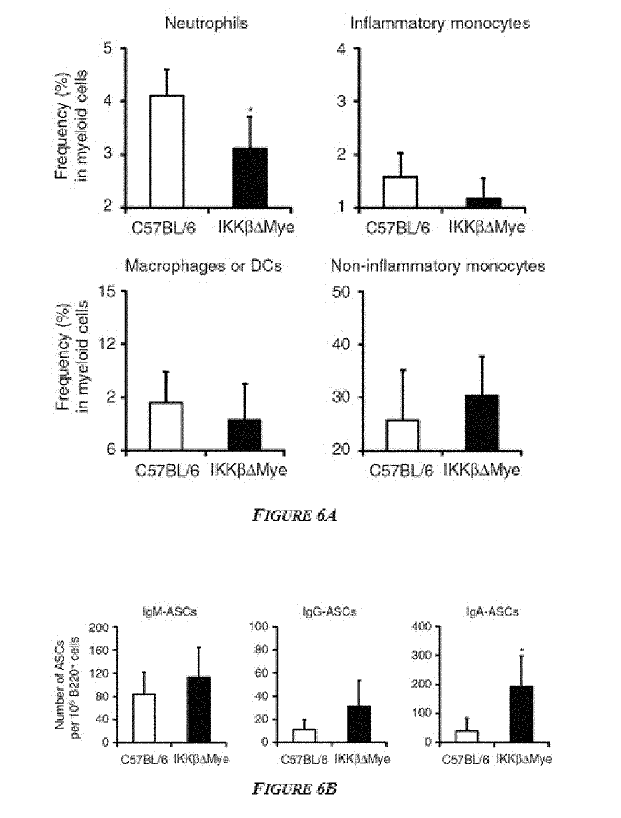

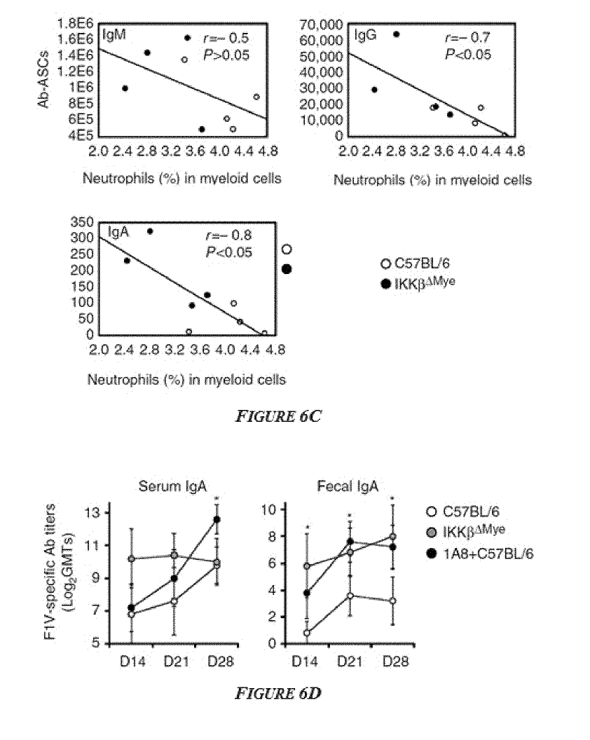

CLNs are considered inductive sites for adaptive immune responses after sublingual and nasal immunization. 6 h after application of EdTx, the frequency of CD11b⁺ cells returned toward basal levels in sublingual tissues (FIG. 2B). Cells may have migrated to CLN and thus myeloid cell subsets in these lymphoid tissues were analyzed. The frequency of neutrophils was significantly reduced in CLNs of IKKβ^{ΔMye} compared to control C57BL/6 mice, while the other myeloid cell subsets remained unchanged (FIG. 6A). CLN cells from EdTx-treated mice were then cultured in the presence of lipopolysaccharide. Three days later, a significantly higher number of IgA-secreting cells in IKKβ^{ΔMye} than in control C57BL/6 mice (FIG. 6B). Of interest, the number of IgA-secreting cells in the CLNs of both control C57BL/6 and IKKβ^{ΔMye} mice were inversely correlated (r=−0.8) with the numbers of neutrophils in these tissues (FIG. 6C).

Reduction of Neutrophils Augments the Adjuvant Effect of EdTx on Ag-specific IgA Responses

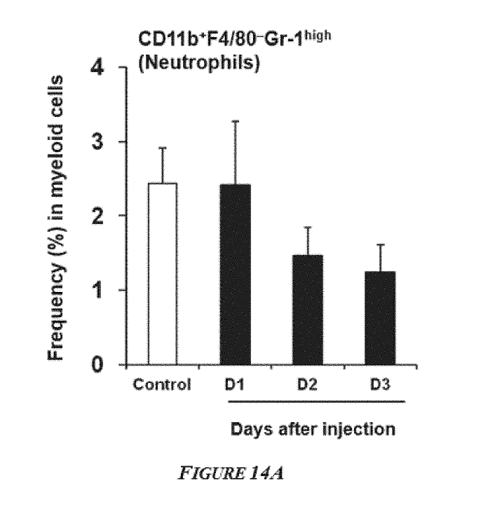

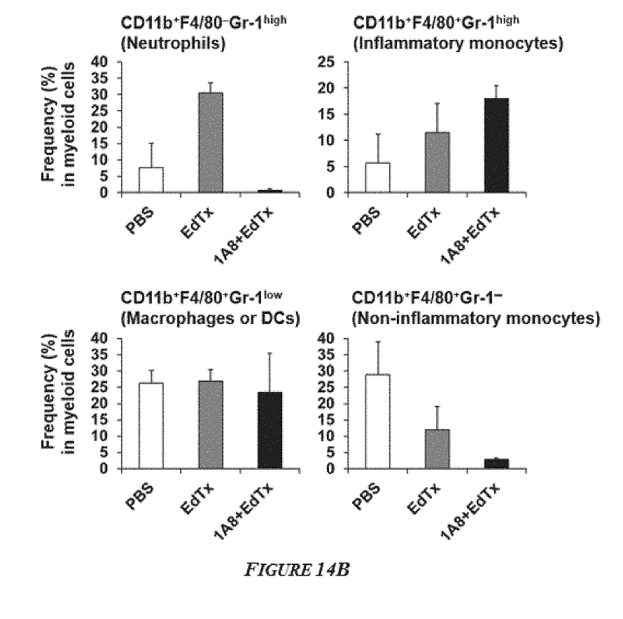

To further establish that an inverse correlation exists between the frequency of neutrophils in sublingual tissues and CLNs, and antigen-specific IgA responses, wild-type C57BL/6 were injected (IP) with a neutrophil Ly6G-specific 1A8 monoclonal Ab 2 days before sublingual immunization with F1-V and EdTx as an adjuvant. This treatment reduced the frequency of neutrophils in CLNs of wild-type C57BL/6 mice (FIG. 14A), and C57BL/6 mice pre-treated with 1A8 (1A8+C57BL/6) contained virtually no neutrophils in sublingual tissues after application of EdTx (FIG. 14B). These 1A8+C57BL/6 mice also gradually developed F1-V-specific serum IgA titers over the time points tested and reached higher serum IgA titers than non-treated C57BL/6 or IKKβ^{ΔMye} mice at Day 28 (FIG. 6D). Interestingly, depletion of neutrophils also enhanced mucosal IgA Ab responses; 1A8+C57BL/6 mice produced high levels of F1-V-specific fecal IgA Abs, which were comparable to those measured in IKKβ^{ΔMye} mice (FIG. 6D).

Neutrophils Suppress Production of IgA by B Cells

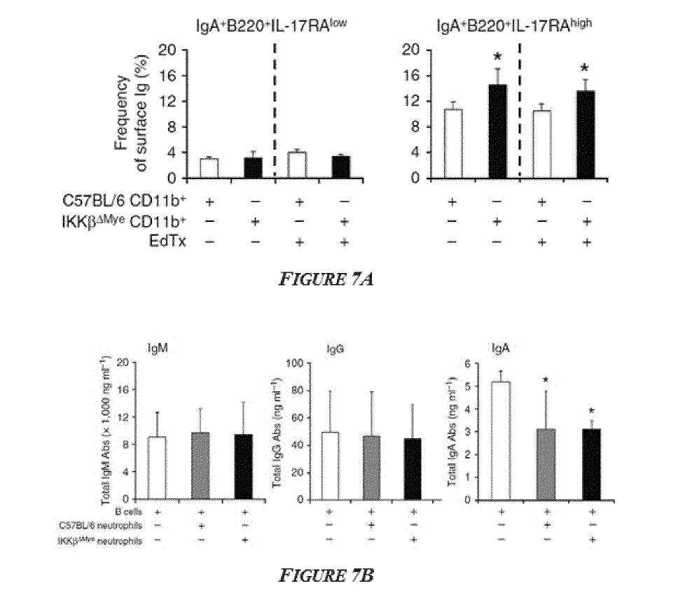

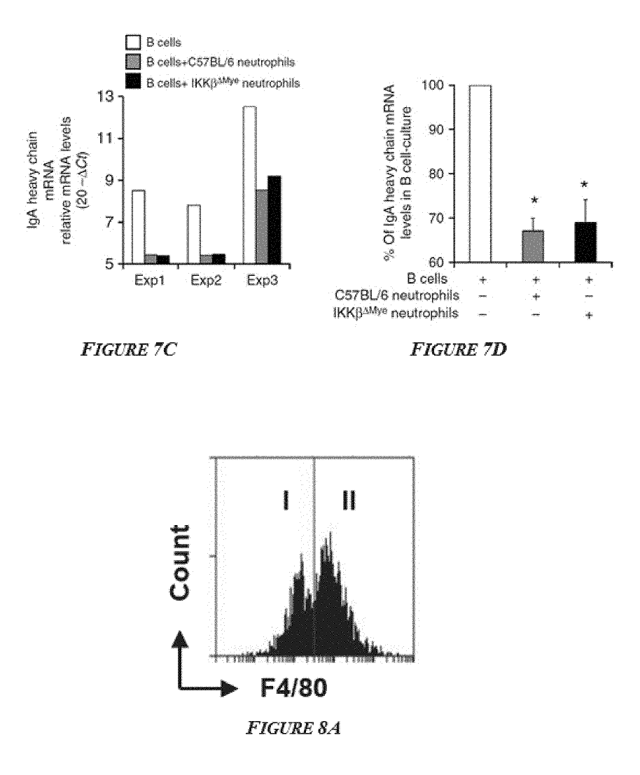

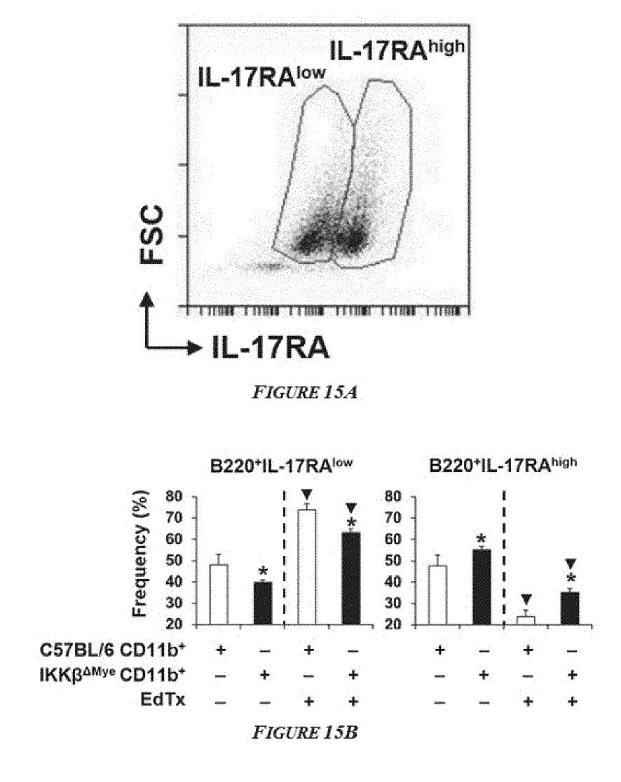

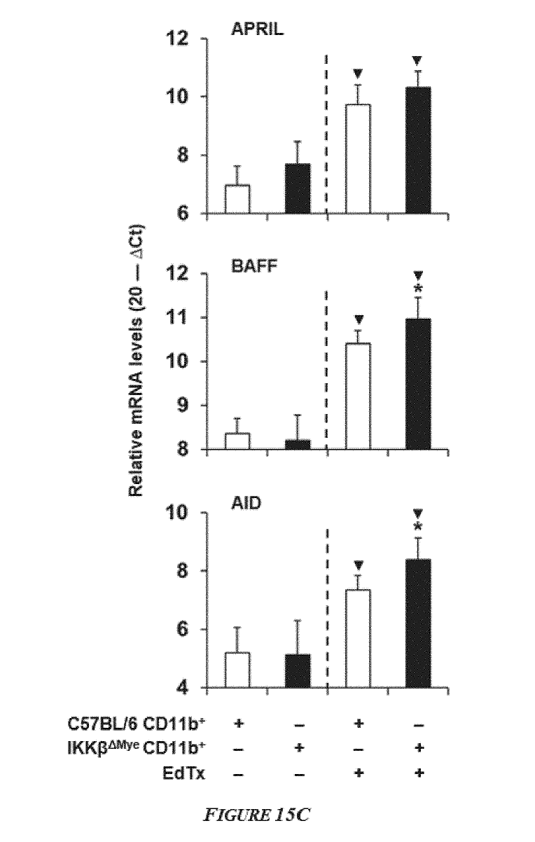

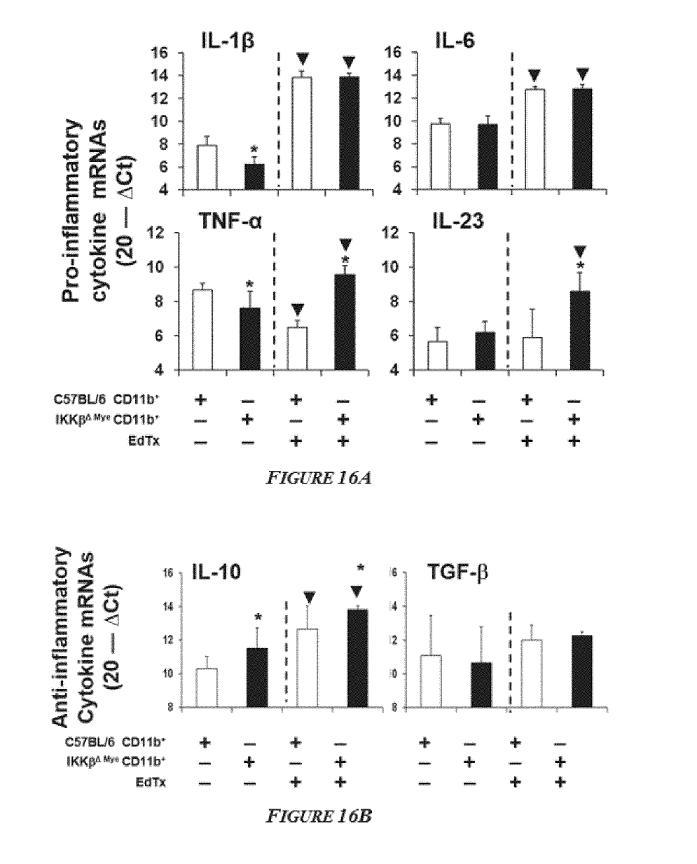

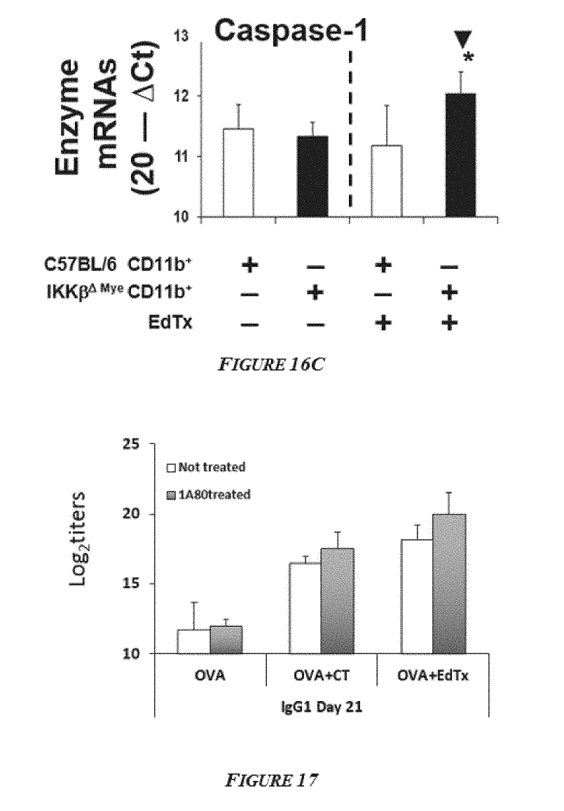

Results clearly show that the ability to generate IgA responses is enhanced in the absence of IKKβ in myeloid cells or when the number of neutrophils is reduced. In addition, the ability of EdTx to induce systemic and mucosal IgA responses in IKKβ^{ΔMye} mice is associated with increased Th17 responses and production of IL-17A (FIG. 5). Thus, experiments were conducted to determine how alteration of canonical NF-κB-mediated signaling via IKKB-deletion in myeloid cells (IKKβ^{ΔMye}) could support Ig class switch and Ab production by B cells. For this purpose, CD11b-depleted spleen cells from C57BL/6 mice were co-cultured with 20% autologous CD11b⁺ cells (C57BL/6 CD11b⁺) or CD11b⁺ cells from IKKβ^{ΔMye} mice (IKKβ^{ΔMye} CD11b⁺) with or without EdTx in the presence of lipopoly-saccharide (Lycke, N., et al. J. Immunol. 142, 3781-3787 (1989)). After 5 days of culture, cells were segregated into IL-17RA^{low} and IL-17RA^{high} cells (FIG. 15A). Co-culture with IKKβ^{ΔMye} CD11b⁺ cells significantly increased the frequency of B220⁺ IL-17RA^{high} B cells (FIG. 15B). As shown in FIG. 7A, these cultures contained low frequencies of surface IgA cells among IL-17RA^{low} B cells regardless of the presence of IKKβ^{ΔMye} CD11b^{|} cells. Interestingly, high frequencies of IL-17RA^{high} B cells expressed surface IgA and co-culture with IKKβ^{Mye} CD11b⁺ cells further increased these frequencies. To further elucidate signals that supported IgA responses, mRNA levels of the B-cell activators a proliferation-inducing ligand (APRIL) and B-cell activation factor of the TNF family (BAFF), and activation-induced deaminase (AID) were analyzed. Addition of EdTx to cultures of spleen cells enhanced mRNA levels of APRIL, BAFF, and AID, and the presence of IKKβ^{ΔMye} CD11b [symbol] further enhanced BAFF- and AID-specific mRNA expression (FIG. 15C). The presence of IKKβ^{ΔMye} CD11b⁺ did not affect EdTx-induced IL-1(3 and IL-6 mRNA levels, but increased EdTx-induced TNF-α, IL-23, IL-10, and Caspase-1 mRNA levels (FIG. 16). Taken together, these results show that myeloid cells lacking IKKβ provide a microenvironment favorable for Ig class switch and B-cell production of IgA.

To gain insight into the mechanism of how neutrophils affect IgA responses, B cells from C57BL/6 mice were co-cultured with or without neutrophils from C57BL/6 or IKKβ^{ΔMye} mice for 5 days. The addition of neutrophils from either C57BL/6 or IKKβ^{ΔMye} mice to cultures of B cells did not affect the secretion of IgM or IgG Abs into culture supernatants (FIG. 7B). Interestingly, co-culture with neutrophils significantly reduced the amounts of IgA Abs secreted by B cells and this inhibitory effect was independent of the presence of functional IKKβ in neutrophils (FIG. 7B). Finally, mRNA analysis of B cells co-cultured with neutrophils showed that neutrophils reduced the level of IgA heavy chain transcripts in B cells (FIG. 7C).

Discussion

Recent studies have identified sublingual immunization as a potentially safer alternative to nasal immunization. However, inductive sites for generating immune responses to sublingual immunization, the identity and function of the cells involved, and the signaling pathways for induction of SIgA via this mucosal route are poorly understood. Disclosed herein is the ability of a sublingual vaccine to mount a SIgA response inversely correlates with the presence of neutrophils in sublingual tissue and CLNs. Depletion of Ly6G⁺ cells improves the development of IgA responses after sublingual immunization and that neutrophils impair the transcription of IgA heavy chain in B cells. This work also shows that myeloid cells lacking IKKβ-dependent NF-κB signaling provide an environment that supports the production of IgA by B cells.

Alum is the most widely used adjuvant for injected vaccines. However, attempts to include alum in mucosal vaccines aimed at prompting SIgA responses have been unsuccessful because this adjuvant fails to effectively induce IgA (Jackson, E. M., et al. PLoS One 7, e41529 (2012)). Studies that addressed mechanisms underlying the adjuvant activity of alum have shown that alum acts via Gr-1 splenic myeloid cells expressing IL-4 to stimulate early B-cell priming (Jordan, M. B., et al. Science 304, 1808-1810 (2004)). Other studies have shown that the NALP3 inflammasome was a crucial element in the adjuvant activity of alum by promoting the maturation of inflammatory cytokines (Eisenbarth, S. C., et al. Nature 453, 1122-1126 (2008)); and furthermore, alum recruits inflammatory monocytes (Kool, M., et al. J. Exp. Med. 205, 869-882 (2008)). In other studies, intranasal co-administration of human neutrophil proteins enhanced antigen-specific serum IgG responses, but failed to promote SIgA responses (Lillard, J. W., et al. Proc. Natl Acad. Sci. USA 96, 651-656 (1999)). These reports are consistent with the disclosed finding that less recruitment of neutrophils into sublingual tissues and CLNs of IKKβ^{ΔMye} mice is a reliable indication of the ability of EdTx as an adjuvant to promote SIgA responses. Because IgG production is not impaired by the recruitment of neutrophils, it is unlikely that neutrophils limit SIgA responses by limiting antigen access to antigen-presenting cells or interactions between the latter and T cells as was previously suggested (Yang, C. W., et al. J. Immunol. 185, 2927-2934 (2010)). Induction of SIgA is well-known to require priming of effector cells in unique inductive sites. Thus, the disclosed finding, that the low proportion of Gr-1⁺ inflammatory monocytes and/or higher proportion of Gr-1⁻ non-inflammatory monocytes in the sublingual tissue correlates with induction of broad Ab responses consisting of both serum IgG and SIgA responses, is in agreement with the recent report that neutrophils also control the spread of T-cell responses to distant lymph nodes (Yang, C. W., et al. J. Exp. Med. 210, 375-387 (2013)). The Gr⁻ monocytes, also described as tissue resident myeloid cells, have been classified as alternatively activated macrophages (M2 macrophages) capable of producing IL-10 and TGF-β (Geissmann, F., et al. Immunity 19, 71-82 (2003); Martinez, F. O., et al. Annu. Rev. Immunol. 27, 451-483 (2009)). Interestingly, these two cytokines are central for Ig class switch in B cells and for production of IgA.

Experiments using IKKβ^{ΔMye} mice provided new insights into signaling for the induction of SIgA responses. Previous studies have shown that the NF-κB pathway can mediate both pro- and anti-inflammatory effects (Greten, F. R., et al. Cell 130, 918-931 (2007); Lawrence, T., et al. Nat. Med. 7, 1291-1297 (2001)). The disclosed data suggest that activation of IKKβ-NF-κB signaling in myeloid cells may in fact reduce their capacity to help B cells undergo Ig class switch for production of IgA. This finding is interesting in light of the recent report that the kinase TBK1 in B cells limits IgA class switch by negative regulation of the non-canonical NF-κB pathway (Jin, J., et al. Nat. Immunol. 13, 1101-1109 (2012)). Thus, stimulation of non-canonical NF-kB signaling either directly in B cells or in other antigen-presenting cells could represent a major pathway for induction of IgA Abs. In this regard, IKKβ deficiency in intestinal epithelial cells increases IgA responses induced by CT used as an oral adjuvant (Bonnegarde-Bernard, A., et al. Mucosal Immunol. 7, 257-267 (2014)). The notion that IKKβ can reduce or suppress the functions of macrophages or DCs is consistent with previous studies by others, suggesting that IKKβ may suppress activation of M1 macrophages during infections through inhibition of STAT-1 (Fong, C. H., et al. J. Exp. Med. 205, 1269-1276 (2008)). In those studies, deletion of IKKβ in macrophages increased STAT-1 activation and promoted a shift toward the M1 phenotype, characterized by increased production of pro-inflammatory and inflammatory cytokines, i.e., IL-1β, TNF-α, IL-12 and IFN-γ, and iNOS in response to IP injection of Group B streptococcus or E. coli lipopolysaccharide (Greten, F. R., et al. Cell 130, 918-931 (2007); Fong, C. H., et al. J. Exp. Med. 205, 1269-1276 (2008)). While the disclosed studies showed enhanced antigen-specific Th1 cytokine responses in IKKβ^{ΔMye} mice after sublingual immunization, the most striking observation was the enhanced IL-17 response.

The IKKβ^{ΔMye} mice were useful tools that helped identify the repressive effect of neutrophils on IgA responses. Analysis of chemokine receptors expression by myeloid cell subsets in sublingual tissues revealed a broader expression of CCR2, CXCR2, and LTB4R2 on macrophages/dendritic cell and non-inflammatory monocytes from IKKβ^{ΔMye} mice. One can speculate that this pattern of receptor expression could improve cellular responses to corresponding ligands and facilitate migration to inductive sites and support IgA responses. Injection of alum recruits neutrophils and induces the formation of nodules consistent with those of extracellular DNA traps (Munks, M. W., et al. Blood 116, 5191-5199 (2010)). A recent report showed that the formation of neutrophil extracellular traps requires phosphorylation of p65 NFkB (Lapponi, M. J. et al. J. Pharmacol. Exp. Ther. 345, 430-437 (2013)). However, neutrophil extracellular traps are primarily known to be involved in the killing of pathogens (Branzk, N., et al. Semin. Immunopathol. 35, 513-530 (2013)). Furthermore, the disclosed results showing that neutrophils from both wild-type and IKKβ^{ΔMye} mice suppress transcription of IgA heavy chain suggest the involvement of other mechanisms.

Nasal immunization with the cyclic AMP-inducing adjuvant CT (Datta, S. K., et al. Proc. Natl Acad. Sci. USA 107, 10638-10643 (2010)) or E. coli heat LT-I (Brereton, C. F., et al. J. Immunol. 186, 5896-5906 (2011)) promotes Th17 responses. EdTx as a sublingual adjuvant promotes antigen-specific Th17 responses in spleen, and is associated with in vitro induction of IL-1β and IL-6. The hallmark cytokine produced by Th17 cells is IL-17A (Korn, T., et al. Annu. Rev. Immunol. 27, 485-517 (2009)). Unlike most Th-derived cytokines, IL-17A does not activate JAK-STAT (Gaffen, S. L., et al. Nat. Rev. Immunol. 9, 556-567 (2009)), but engages Act1 leading to activation of IKKβ and downstream NF-κB, C/EBP, and AP-1, which in turn lead to expression of pro-inflammatory cytokines (Maitra, A., et al. Proc. Natl Acad. Sci. USA 104, 7506-7511 (2007); May, M. J. Nat. Immunol. 12, 813-815 (2011)). CD11b^{|} cells from IKKβ^{ΔMye} mice increase specific B-cell populations, i.e., IL-17RA^{high} B cells, and the IL-17RA^{high} B cells express higher levels of surface IgA. IL-17A was reported to act as a helper for the development of germinal centers (Hsu, H. C., et al. Nat. Immunol. 9, 166-175 (2008)). The disclosed results suggest that IKKβ^{ΔMye} cells stimulate B cells to be more responsive to IL-17A. This pathway could be one of the mechanisms that rescues the mucosal adjuvant EdTx and the induction of SIgA Abs.

The limited understanding of molecular and cellular mechanisms that regulate IgA responses has hampered the development of safe mucosal vaccines capable to promote mucosal IgA production. Using an experimental vaccine adjuvant that does not normally induce SIgA after sublingual immunization, IKKβ is shown to be one of the key regulatory pathways for induction of SIgA responses by sublingual vaccines. Neutrophils were also shown to negatively regulate IgA production by B cells, an effect that can be countered by Gr-1⁻ myeloid cells lacking a functional IKKβ. The disclosed results provide new insights for the development of sublingual vaccines that can promote both IgA at mucosal surfaces and IgG in the blood stream for optimal protection against infectious agents.

### Example 2

### Pharmacological Inhibitors of Neutrophil Function

In this example, the effect of pharmacological inhibitors of neutrophil function was analyzed. These pharmacological inhibitors of neutrophil functions can provide a broad choice of tools that can be incorporated into vaccine formulations in order to induce high levels of IgA responses in mucosal secretions such as saliva, vaginal secretion, the intestinal lumen, and tears. More specifically, the ability of pharmacological agents targeting Gr-1⁺ cells (i.e., neutrophils) to promote IgA production by B cells in vitro was analyzed. Finally, the pharmacological inhibitors that promote IgA production in vitro were tested to determine if they can elicit generalized IgA responses, including in mucosal secretions, when incorporated into injected or needle-free epicutaneous vaccines.

Several Pharmacological Agents Targeting Gr-1⁺ Cell Products/Functions Promote IgA Production by B Cells In Vitro

TABLE 2
Inhibitors of neutrophil function that were tested

| | Inhibitors | | | |
|---|---|---|---|---|
| | Acetyl-salicylic | Sivelestat | | |
| | acid (ASA) | Or ONO 5046 | Sulfasalazine | Roflumilast |
| Function | Cox-2 inhibitor | Neutrophil | Neutrophil NET | PDE4 inhibitor |
| | | elastase inhibitor | inhibitor | |
| Current | Marketed as Aspirin | Marketed as | Marketed as | Marketed as Daxas |
| use | (for pain, fever, | Elaspol | Azulfidine | or Daliresp |
| | inflammation) | (for acute | (for Rheumatoid | (for COPD) |
| | | respiratory | arthritis) | |
| | | failure) | | |

Spleen cells (2×10⁶/ml) of naïve C57BL/6 mice were cultured 5 days in the presence of LPS (0.5 μg/ml) alone or LPS with ASA or specific inhibitors of neutrophil functions. The IgA Abs secreted in culture supernatants were quantified by ELISA.

TABLE 3
Effect of pharmacological inhibitors of neutrophil function on IgA production by B cells

| | Acetyl-salicylic | | Sivelestat | | | | | |
|---|---|---|---|---|---|---|---|---|
| | acid (ASA) | | Or ONO 5046 | | Sulfasalazine | | Roflumilast | |
| Relative stimulation | ++ | | ++ | | +++ | | + | |
| of IgA secretion | | | | | | | | |
| Dose effect | 10 | uM (++) | 4.6 | uM (+) | 10 | uM (++) | 0.062 | uM (−) |
| (Relative stimulation | 100 | uM (++) | 46 | uM (++) | 100 | uM (+++) | 0.62 | uM (−) |
| of IgA) | 500 | uM (+) | 230 | uM (++) | 1000 | uM (+) | 6.2 | uM (+) |

Relative stimulation of IgA secretion is expressed as the relative level of IgA secreted when compared with cells cultured in the presence of LPS alone.
The neutrophil elastase inhibitor Sivelestat promotes IgA responses by a sublingual vaccine.

Groups of wild-type C57BL/6 mice were immunized three times at weekly intervals by sublingual application of Yersinia pestis F1-V antigen (50 μg) either alone or in the presence of an edema toxin derivative (15 EdTx), or EdTx plus 100 uM Sulfasalazine. F1-V-specific IgA Ab responses in serum and fecal samples were analyzed by ELISA and end-point titers were expressed as Log₂ GMTs. ±SD. * p≤0.05 compared with C57BL/6 in each day (n=5).

TABLE 4
Effect of a pharmacological inhibitor of neutrophil
function on IgA responses to a sublingual vaccine.

| | Serum IgG | Serum IgA | Saliva IgA | Fecal IgA |
|---|---|---|---|---|
| F1V alone | 21 ± 0.8 | <5 | <2 | <2 |
| F1V + EdTx | 21 ± 0.1 | <5 | <2 | <2 |
| F1V + EdTx + | 22 ± 0.8 | 11 ± 0.6 | 9 ± 0.5 | 6 ± 0.5 |
| Sulfasalazine | | | | |

### Example 3

### Effect of Neutrophil-Depletion on Antigen-Specific IgA Responses to Epicutaneous Vaccination

In this example, the effect of neutrophil depletion on antigen-specific IgA responses to epicutaneous vaccination was examined. Neutrophils were depleted using the 1A8 mAb, given to C57BL/6 mice i.p. on days −3, 4, and 11. The OVA antigen was given by epicutaneous immunization on intact skin (abdomen) using a volume of 100 μl. Mice were divided into three groups: 1) OVA only (1 mg) 2) OVA+cholera toxin (CT, 25 μg) and 3) OVA+B. anthracis edema toxin (EdTx: 50 μg of PA+50 μg of EF). Antibody responses in serum and mucosal secretion were examined at days 14, 21, and 28.

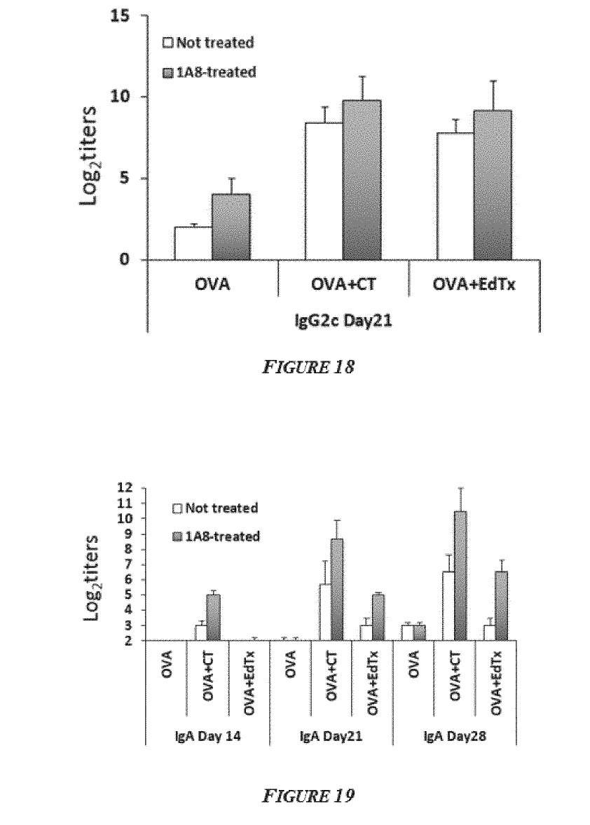

As seen in FIGS. 17 and 18, there was a slight increase in both IgG1 and IgG2c in mice treated with the 1A8 mAb for depletion of neutrophils. There was no change in the IgG1/IgG2c ratio, suggesting there is no major impact on Th2/Th1 responses.

As seen in FIG. 19, depletion of neutrophils with the 1A8 mAb increased levels of OVA-specific serum IgA responses induced by cholera toxin as adjuvant and also led to the development of serum IgA responses in mice immunized with edema toxin as adjuvant.

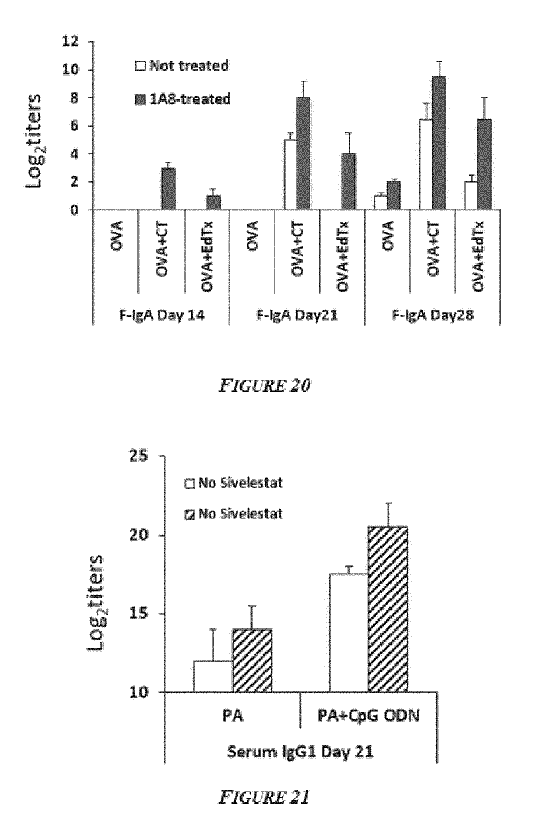

As seen in FIG. 20, depletion of neutrophils with the 1A8 mAb increased levels of fecal IgA (secretory IgA) responses induced by cholera toxin as adjuvant and also led to the development of fecal IgA (secretory IgA) responses in response to edema toxin as adjuvant.

### Example 4

### The Neutrophil Elastase Inhibitor Sivelestat Enhances Systemic IgG and Mucosal IgA Responses Induced by Experimental Vaccine Containing CpG as Adjuvant

A vaccine capable of promoting anti-PA Abs in the bloodstream and in the airways may provide better protection against inhalational anthrax than the current injected alum-adsorbed PA-based vaccines. To examine this, a neutrophil elastase inhibitor (sivelestat) was examined in combination with CpG adjuvant to determine its effect on systemic IgG and mucosal IgA responses. C57BL/6 mice were given a nasal immunization of protective antigen of Bacillus anthracis (PA) (50 μg/dose given at a volume of 15 μl per nostril) three times at weekly intervals (days 0, 7, and 14). CpG adjuvant was administered at 5 μg/dose (CpG ODN 1826).

Mice were divided into four treatment groups: (1) PA only (no Sivelestat) (2) PA+CpG (3) PA+ neutrophil elastase inhibitor (Sivelestat) and (4) PA+CpG+neutrophil elastase inhibitor (Sivelestat). Antibody responses in serum and mucosal secretions were examined at days 14, 21, and 28.

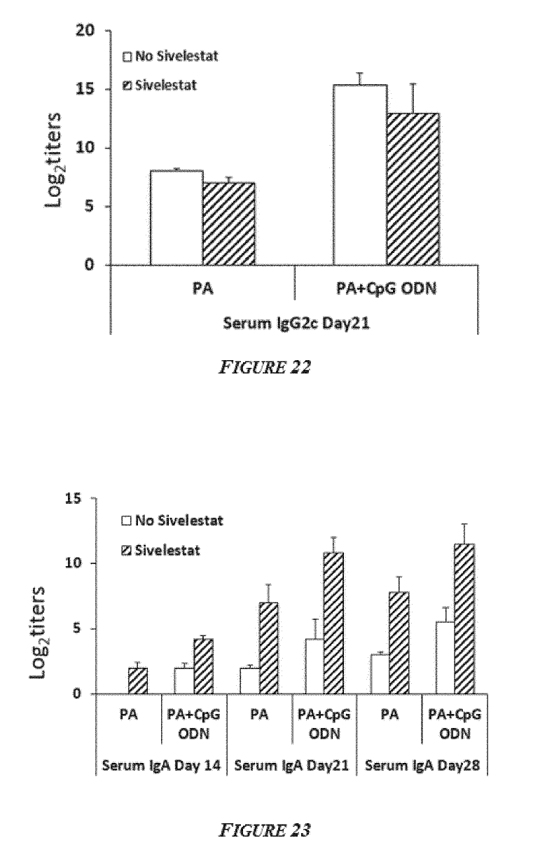

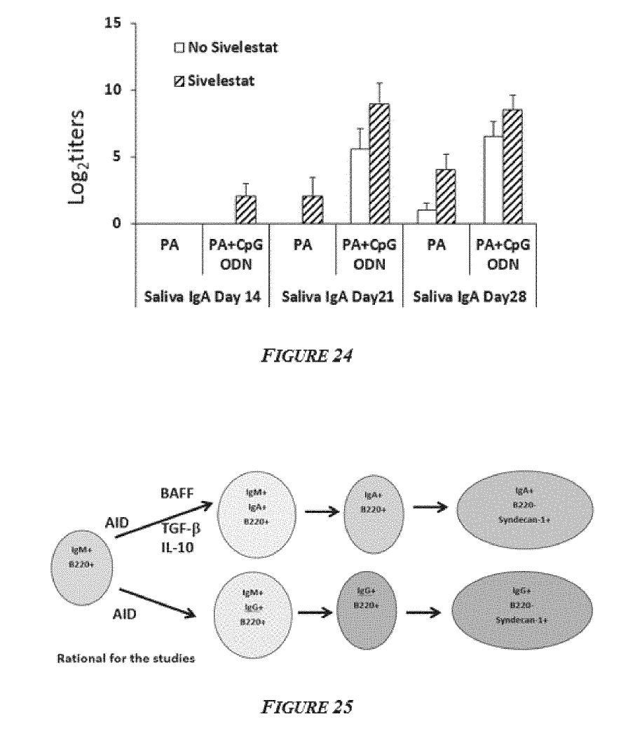

As shown in FIG. 21, sivelestat enhances PA-specific serum IgG1 response. In addition, sivelestat enhances PA-specific serum IgA response (FIG. 23) and PA-specific salivary IgA response (FIG. 24). However, sivelestat did not affect the PA-specific serum IgG2c response (FIG. 22).

### Example 5

### The Different Inhibitors of Neutrophil Function Differentially Regulate Immunoglobulin Class Switching and Production of IgA Responses

In this example, the signaling induced by neutrophil inhibitors were examined to provide insight into mechanisms used to promote immunoglobulin class switching and production of IgG and/or IgA antibodies. In these experiments, the spleen cells of nice mice were cultured 24 hrs without or with neutrophil inhibitor (roflumilast (Phosphodiesterase-4 inhibitor), sivelestat (Neutrophil elastase inhibitor), sulfasalazine (Neutrophil NET inhibitor), or aspirin (COX inhibitor)). Gene expression was analyzed by qRT-PCR for BAFF, AID, TGFb, and IL-10. Mice were divided into 5 groups: (1) Control (Media) (2) Roflumilast (6.5 μM) (3) Sivelestat (46 μM) (4) Sulfasalazine (100 uM) and (5) Aspirin (100 μM).

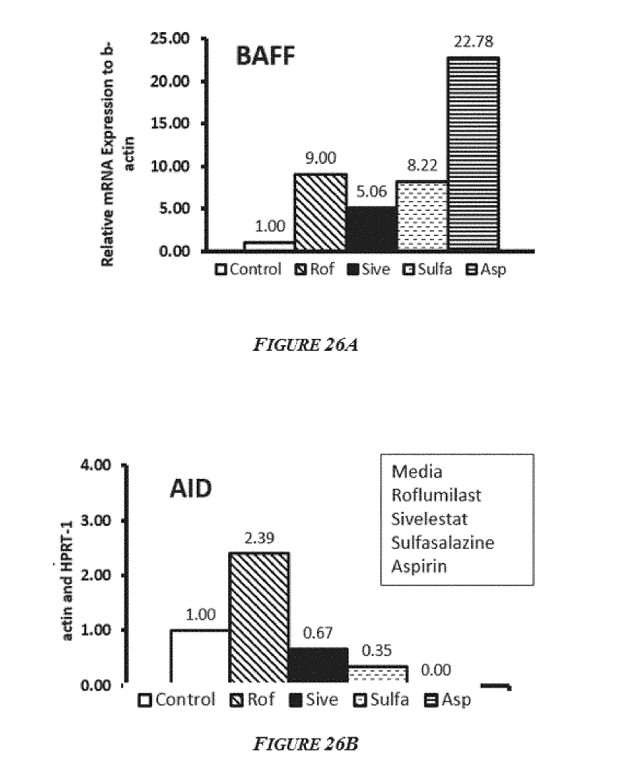

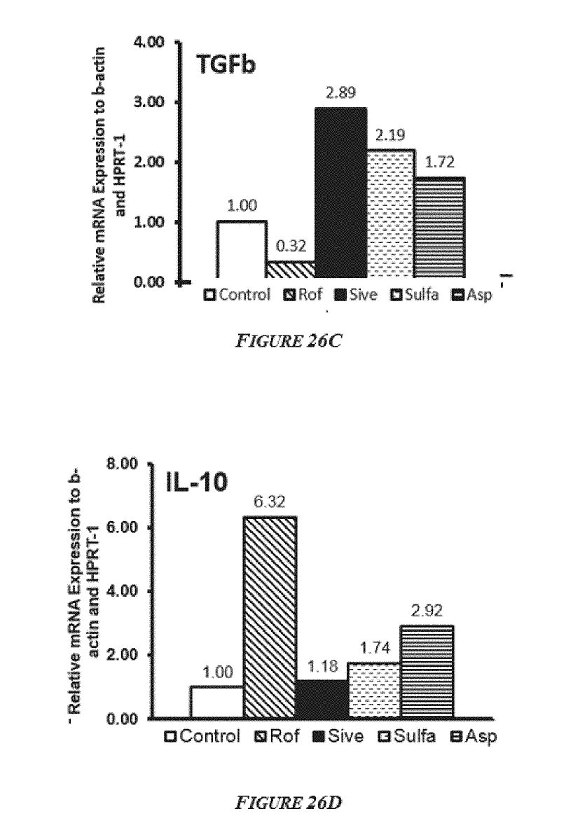

As seen in FIG. 26, the different neutrophil inhibitors tested (roflumilast, sivelestat, sulfasalazine, and aspirin) differentially regulate genes involved in immunoglobulin class switching (BAFF, AID, TGFb, and IL-10).

In additional experiments, in vitro regulation of Ig class switch and IgG and IgA production by neutrophil inhibitors was examined. Spleen cells of nice mice were cultured for 4 days in the presence of cholera toxin B subunit (CT-B) in the presence or absence of a neutrophil inhibitor (roflumilast (6.5 μM), sivelestat (100 μM), sulfasalazine (100 μM), or aspirin (100 μM)). The frequency of IgA secreting cells was analyzed by ELISPOT. Finally, IgG and IgA production were analyzed by immunofluorescence on cytospins.

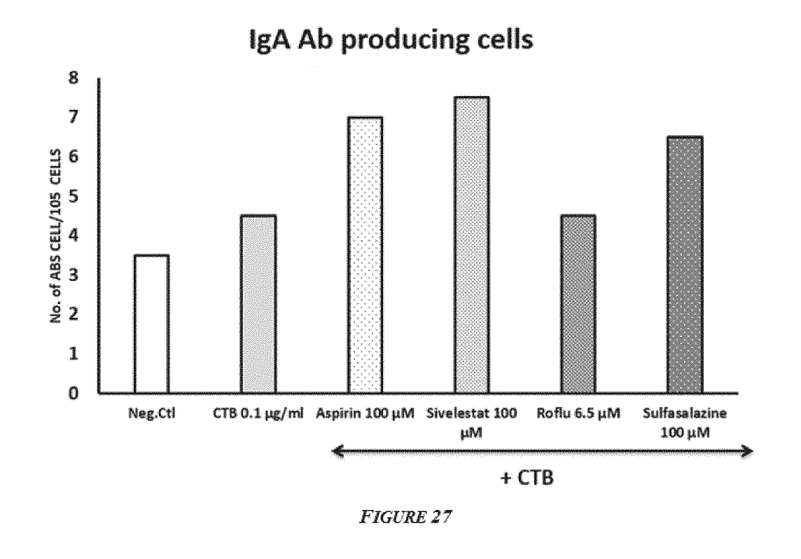

As seen in FIG. 27, the neutrophil inhibitors sivelestat (100 μM), sulfasalazine (100 μM), and aspirin (100 μM)) enhanced IgA secretion by spleen cells in the presence of the adjuvant cholera toxin B (CTB) subunit.

Neutrophil inhibitors enhance IgG and IgA secretion by spleen cells cultured in the presence of the adjuvant cholera toxin B subunit (CTB). Spleen cells of nice mice were cultured for 4 days in the presence of cholera toxin B subunit (CT-B) in the presence or absence of a neutrophil inhibitor (roflumilast (6.5 μM), sivelestat (100 μM), sulfasalazine (100 μM), or aspirin (100 μM)). IgG and IgA production were analyzed by immunofluorescence after staining cytospin samples with FITC-conjugated anti-IgA or PE-conjugated anti-IgG antibodies. Aspirin enhanced IgG and IgA production, while larger increases in IgG and IgA production were seen in spleen cells treated with sivelestat.

### Example 6

### Inhibitors of Elastase Stimulate B Lymphocyte Differentiation into IgG- and IgA-Producing Cells

It is well established that dendritic cells and macrophages play a role in antigen presentation to B and T cells and in shaping B and T cell responses via cytokines they produce. Previous experiments show that depletion of neutrophils improves the production of mucosal IgA after sublingual immunization with Bacillus anthraces edema toxin as adjuvant. These past studies also demonstrated that an inverse correlation exists between the number of neutrophils and production of IgA by B cells. Using specific inhibitors of elastase, the elastase activity of neutrophil was investigated as to whether it interferes with production of IgA and possibly other immunoglobulin isotypes. It was found that murine splenocytes and mesenteric lymph node cells cultured for 5 days in the presence of neutrophil elastase inhibitors secreted higher levels of IgG and IgA than cells cultured in the absence of inhibitor. The effect of the inhibitors was dose-dependent and was consistent with increased frequency of CD138⁺ cells expressing IgG or IgA. Finally, neutrophil elastase inhibitors increased transcription of mRNA for AID, IL-10, BAFF and APRIL, factors involved in B cell differentiation. These findings identify inhibitors of elastase as adjuvants for increasing production of antibodies.

### Background

A series of finely regulated events lead to maturation of B cells and their differentiation into plasma cells that secrete antibodies [1]. Thus, after antigen stimulation of IgM-bearing inactivated B cells, these cells undergo Ig class switch recombination (CSR) to produce antibodies of different isotypes but with the same antigen specificity than the IgM. The activation-induced cytidine deaminase (AID or AICD) plays a crucial role in CSR and somatic hypermutation [2]. Additional signals for immunoglobulin CSR are provided by the ligation of CD40L on B cells and other factors such as B cell-activating factor of the TNF family (BAFF), and a proliferation-inducing ligand (APRIL). Furthermore, an array of cytokines including IL-4, IL-13, IFN-γ, TGF-β, and IL-10 regulate the Ig class switching toward selected isotype and subclasses, while other provide survival and proliferation signals (IL-5, and IL-6), or enhance antibody affinity maturation in the germinal centers (i.e., IL-21) [3]. A number of cells contribute co-stimulatory and cytokines signals required for Ig CSR and production of antibodies by B cells. Macrophages and dendritic cells contribute via their expression of CD40 and secretion of BAFF, APRIL, as well as pro-inflammatory (i.e., IL-6, IFN-γ) and anti-inflammatory (i.e., TGF-β, IL-10) cytokines. Epithelial cells can produce BAFF and APRIL, as well as cytokines, including IL-6 and TGF-β. Cytokines produced by T helper cell, and innate lymphoid cells in mucosal tissues, play an important role in both Ig CSR and affinity maturation. Mast cells produce IL-6 and IL-10 and a mast cell activator compound (i.e., compound 48/80) was shown to promote IgA responses in vivo by stimulating the migration of dendritic cells (DC) into T cell area [4].

Neutrophils represent the largest number of myeloid cells in the blood stream and the major phagocytic cells that eliminate invading pathogens [5,]. An inverse relationship is observed between IgA response and the early recruitment of neutrophils in sublingual tissues and cervical lymph nodes after sublingual immunization with Bacillus anthraces edema toxin as an adjuvant [6]. Neutrophils were also found to suppress IgA production in vitro via mechanisms independent of NF-κB pathway [6]. The primary (or azurophilic) granules of neutrophils contain defensins, myeloperoxidase, lysozymes, and three serine proteases: neutrophil elastase, cathepsin G and protease 3 [5, 7]. Neutrophil elastase (NE) is a cationic glycoprotein stored in readily active form in primary granules at concentrations exceeding millimolar range and thus, making it a major antimicrobial enzyme of neutrophils [8, 9].

It was also investigated whether the elastase activity of neutrophils could mediate their suppressive effect on IgA production. Here, it was shown that inhibitors of NE activity stimulate production of IgG and IgA by spleen and mesenteric lymph node cells in vitro, which indicates that elastase-like serine proteases modulate Ig CSR by B cells and production of antibodies.

### Results and Discussion

Neutrophil Elastase Inhibitors Stimulate Production of IgG and IgA.

NE contributes to tissue destruction in inflammatory diseases including emphysema, glomerulonephritis, rheumatoid arthritis [10] and colitis [11]. NE also exacerbates lung pathologies via stimulation of goblet cell metaplasia and mucus production [12]. The latter effect of NE was shown to be associated with stimulation of cytokine responses (i.e., KC and IL-5) and recruitment of lymphocytes and eosinophils in the airways [12]. Another study has shown that NE regulates lung inflammatory responses after infection with P. aeruginosa, and that purified NE stimulates IL-6, TNF-α and MIP-2 responses by murine macrophages [13]. The effects of NE extend beyond regulation of cytokine responses, because uptake of NE by breast cancer cells upregulates their MHC class I molecule expression and enhances the presentation of tumor-associated antigen [14].

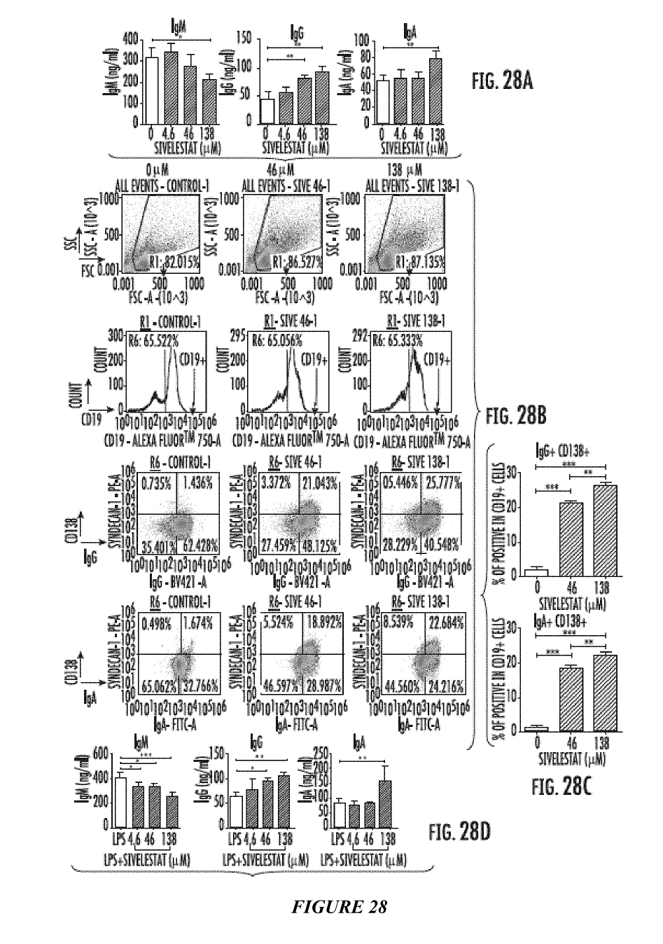

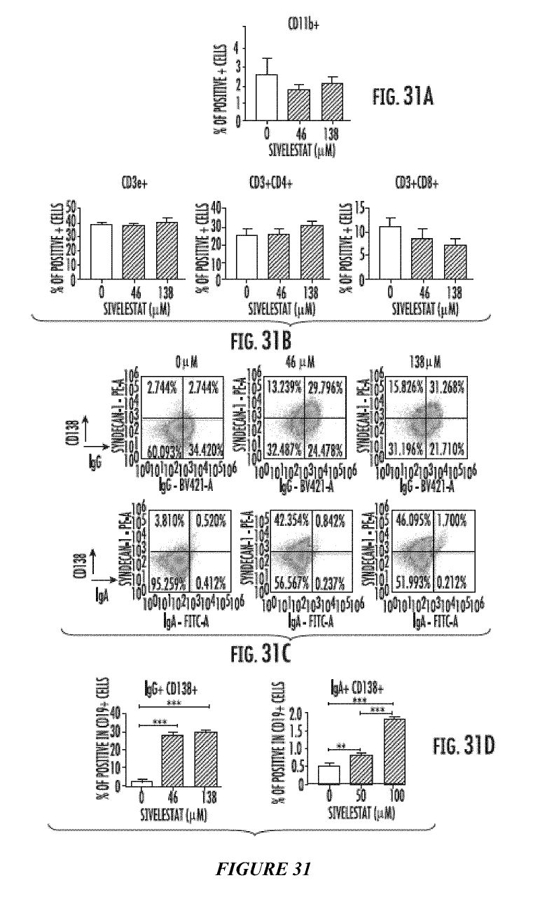

To address whether NE regulates immunoglobulin production, spleen cells were cultures for 5 days in the presence of doses of the Sivelestat (ONO-5046), a potent and inhibitor of neutrophil elastase [15]. Analysis of immunoglobulin isotypes in culture supernatants showed that Sivelestat reduced secretion of IgM in a dose-dependent manner (FIG. 28A). This effect more likely reflected a stimulation of immunoglobulin class switching because it correlated with a dose-dependent increase of IgG and IgA secretion (FIG. 28A). In order to elucidate how the inhibitor stimulated secretion of immunoglobulins, splenocytes were analyzed by flow cytometry after 2 days of culture. The presence of Sivelestat did not affect the frequency of myeloid cells (CD11b⁺) FIG. 31A), T cell subsets (FIG. 31B) or B cells (CD19⁺) (FIG. 28B). Interestingly, this inhibitor increased the frequency of CD138⁺ among CD19⁺ (FIGS. 28B, 31C, and 31D). The CD138⁺ CD19⁺ cells induced by Sivelestat expressed surface IgG or a low but significant percentage expressed surface IgA (FIG. 31D). Intracellular cellular staining of immunoglobulin isotypes confirmed induction of CD138⁺CD19⁺IgG⁺ and CD138⁺CD19⁺IgA⁺ cells by Sivelestat (FIGS. 28B and 28C). Finally, addition of Sivelestat also increased secretion of IgG and IgA by splenocytes cultured in the presence of LPS (FIG. 28D).

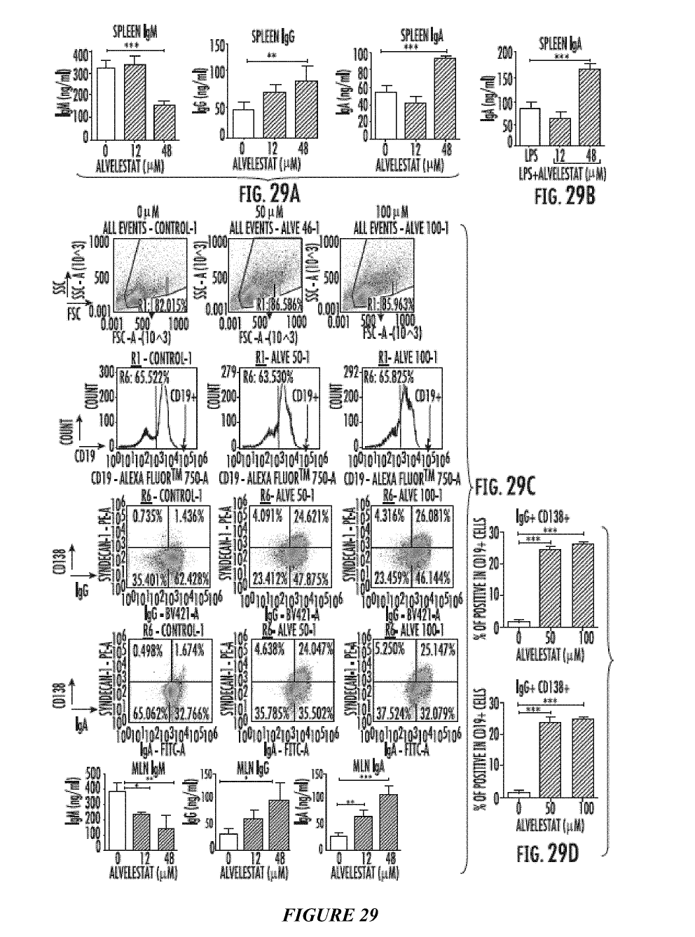

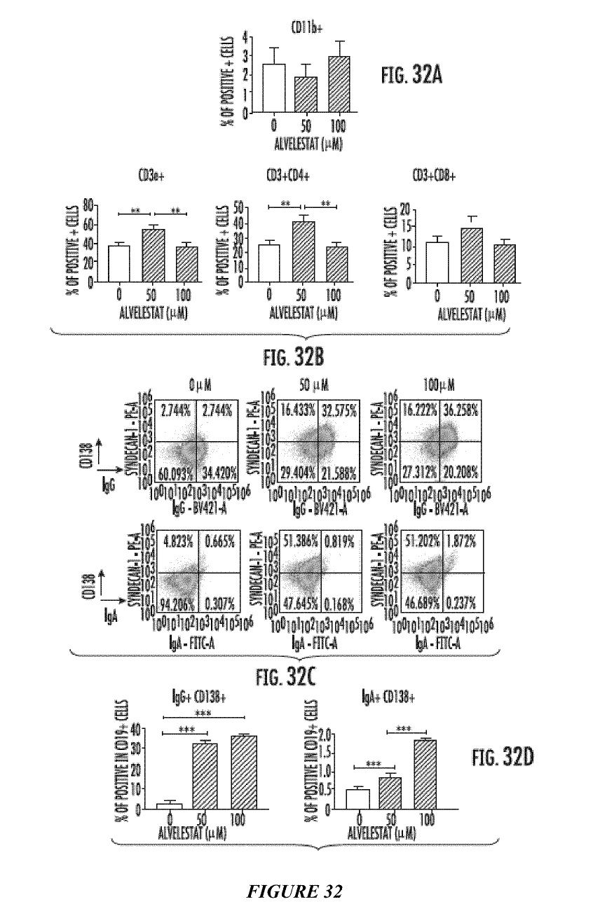

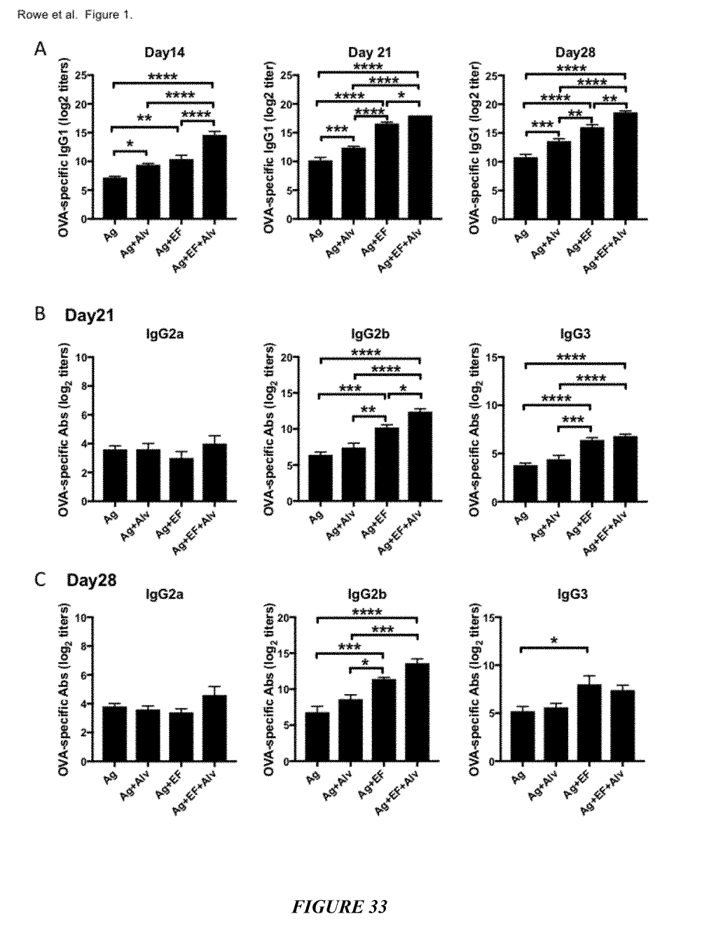

To confirm that the effect of Sivelestat on IgG and IgA production was a characteristic of elastase inhibitors, Alvelestat (AZD9668), a fluorinated inhibitor of NE, was evaluated. The presence of Alvelestat reduced IgM and increased IgG and IgA secreted by splenocytes in vitro (FIG. 29A). This effect was dose-dependent and significant increase in IgA production was also seen when Alvelestat was added to spleen cells cultured in the presence of LPS (FIG. 29B). Next, the cellular changes induced by Alvelestat to support IgG and IgA production were analyzed. The frequencies of CD11b⁺ (FIG. 32A) or CD19⁺ spleen cells (FIG. 29C), were not affected after 2 days culture in the presence of Alvelestat. This treatment however, increased the frequency of CD4+ T cells (FIG. 32B). Of interest for antibody secretion, Alvelestat increased the frequency of CD138⁺ CD19⁺ cells, which expressed surface IgG or a low but significant percentage of surface IgA (FIGS. 29C, 32C and 32D). Production of IgA or IgA by Alvelestat-induced CD138⁺CD19⁺ was further confirmed by intracellular cellular staining of immunoglobulin isotypes (FIGS. 29C and 2D). It was also established that the regulatory effects of NE inhibitors on B cell differentiation and IgG and IgA production were not limited to spleen cells. Thus, similar patterns of response were seen in mesenteric lymph node cells cultured in the presence of Alvelestat, which produced higher levels of IgG and IgA in culture supernatants (FIG. 29E).

These findings are in line with a previous report that depletion of neutrophils before systemic injection of vaccines increased the magnitude of antigen-specific CD4⁺ T cell responses, and the levels of antigen-specific serum IgG responses [16]. These authors showed that neither reactive oxygen species, nor nitric oxide, were involved in the suppressive effect of neutrophils. It should be noted that their study identified antigen presentation by macrophages and dendritic cells as the step limited by neutrophils and that neutrophils could mediate this effect in the absence physical interactions with dendritic cells or T cells [16], suggesting that factors secreted by neutrophils were responsible for the effect. These findings that inhibition of NE activity stimulates secretion of IgG and IgA indicates that elastase is the molecule (or one of the molecules) secreted by neutrophils that impairs antibody production, and perhaps adaptive immunity. This notion is consistent with the report that increased levels of NE in hematopoietic stem cells and progenitor cells in the bone marrow of mice fed high-fat diet was associated with reduced B lymphocytes production [17].

The Neutrophil Elastase Inhibitors Stimulate Cytokine Signals for Ig Class Switch Recombination

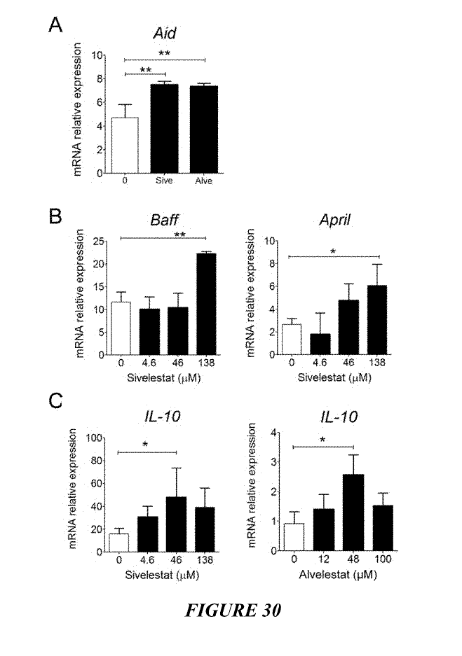

To gain insight into mechanisms used by NE inhibitors to facilitate production of IgG and IgA, the expression of AID, BAFF and APRIL, and IL-10 transcripts were analyzed in spleen cells cultured 24 hrs in the presence of NE inhibitors. Both Alvelestat and Sivelestat were found to stimulated AID mRNA synthesis (FIG. 30A). The NE inhibitors also induced expression of BAFF, APRIL (FIG. 30B) and IL-10 mRNA in a dose-dependent manner (FIG. 30C).

Neutrophil elastase and the related serine proteases cathepsin G and proteinase 3, were shown to regulate cytokine responses through activation or degradation of cytokines, cytokine receptors, or toll-like receptor [5, 18]. This is consistent with the finding that NE inhibitors stimulate IL-10 expression, but also BAFF and APRIL in cultures of splenocytes. IL-10 was reported to regulate expression of AID for induction of Ig CSR [19] and it is well-established that IL-10 facilitates Ig class switching for production of IgA [20]. Thus, this data suggests that NE inhibitors stimulate antibody production through stimulation of AID and inhibition of the activation/degradation of cytokines and cytokine receptors by elastase. NE was also shown to inhibit maturation and function of dendritic cells, including expression of the costimulatory molecules CD40, CD80 and CD86 [21]. Thus, the presence of neutrophil inhibitors may have promoted the maturation of dendritic cells and their expression BAFF and APRIL and perhaps costimulatory molecules (i.e., CD40) for enhanced antibody production.

### Conclusion

It was previously shown that neutrophil peptide defensins enhance IgG, but not IgA responses against nasally co-administered vaccine antigens [23]. Here, it was have shown that the serine protease activity of neutrophil elastase, and possibly other serine protease such as B cell elastase, also participate in the regulation of B cell biology and adaptive immunity. However, unlike neutrophil peptide defensins, NE negatively regulate differentiation of B lymphocytes into IgG and IgA secreting cells. It was previously reported that depletion of neutrophils enhances IgA responses to sublingual immunization with Bacillus anthracis edema toxin as adjuvant [6]. This finding here shows that inhibitors of serine proteases are an alternative to neutrophil depletion as a strategy for enhancing IgG and IgA responses.

### Material and Methods

Mice

C57BL/6 mice were obtained from The Jackson Laboratory (Bar Harbor, Me.) and were used at 8-12 weeks of age. All experiments were in accordance with both National Institutes of Health and Institutional Animal Care and Use Committee guidelines to avoid pain and destress.

In vitro cultures

Single cell suspensions of spleen cells, mesenteric lymph nodes were resuspended (2×10⁶ cells/ml) in RPMI 1640 supplemented with L-glutamine, HEPES, penicillin, streptomycin and 10% heat-inactivated fetal calf serum. Cells were cultured for 1, 5, or 6 days at 37° C. and 5% CO₂ in the presence of various doses of the neutrophil elastase inhibitor Sivelestat (ONO-5046, C₂₀H₂₂N₂O₇S) or Alvelestat (AZD9668, C₂₅H₂₂F₃N₅O₄S) (Selleckchem, Houston, Tex.). In selected experiments, neutrophil elastase inhibitors were added to cells cultured in the presence of Salmonella typhimurium LPS (0.05 μg/ml, Sigma, Saint Louis, Mo.).

Real-Time RT-PCR

For analysis of mRNA responses, cells were collected after 1-day culture and washed once in RPMI before addition of TRIzol (Invitrogen, Carlsbad, Calif.). RNA was isolated, and cDNA was synthesized by using Superscript III (Invitrogen). Real-time PCR was performed as previously described [24] and data were expressed as relative mRNA expression=2^{-ΔΔCt} where ΔCt=Ct_{unknown}−Ct_{HKG}, and normalized against two house-keeping genes (HKG): β-actin and HPRT1.

Flow Cytometry Analysis

Cell suspensions were collected after 2 days of culture and stained with combinations of the following Abs: APC anti-CD11b (eBioscience), AF700 anti-CD3e, AF750 anti CD4, PerCp-Cy5.5 anti-CD8a, BV421 anti-IgG, AF750 anti-CD19 (Biolegend), PE anti-CD138 (Syndecan-1, R&D Systems), FITC anti-IgA (Southern Biotech). For intracellular detection of IgG and IgA, golgi-stop was added to cell cultures 4 hours before harvest and extracellular staining with anti-CD19 and anti-CD138 Abs. Cells were then fixed and permeabilized before intracellular staining with anti-IgG and anti-IgA Abs. Stained cells were analyzed with an Attune NxT flow cytometer (Thermo Fisher Scientific, Waltham, Mass.).

Immunoglobulin Isotype ELISA

The amount of immunoglobulin isotype secreted in culture supernatants after 6 days of culture was measured as previously described [25] by ELISA, using goat anti-murine IgM, IgG, and IgA antibodies (Southern Biotech, Birmingham, Ala.) and extrapolation against standards.

Statistical Analysis

Results were expressed as mean±one standard deviation. Statistical significance was determined by ANOVA. Results were considered significant at p<0.05. Statistical tests were performed using GraphPad Prism 6 (GraphPad Software Inc, La Jolla, Calif.).

### References Cited in this Example

[1] M. Shapiro-Shelef, K. Calame, Regulation of plasma-cell development, Nat Rev Immunol, 5 (2005) 230-242.
[2] M. Muramatsu, K. Kinoshita, S. Fagarasan, S. Yamada, Y. Shinkai, T. Honjo, Class switch recombination and hypermutation require activation-induced cytidine deaminase (AID), a potential RNA editing enzyme, Cell, 102 (2000) 553-563.
[3] E. V. Acosta-Rodriguez, M. C. Merino, C. L. Montes, C. C. Motran, A. Gruppi, Cytokines and chemokines shaping the B-cell compartment, Cytokine Growth Factor Rev, 18 (2007) 73-83.
[4] J. B. McLachlan, C. P. Shelburne, J. P. Hart, S. V. Pizzo, R. Goyal, R. Brooking-Dixon, H. F. Staats, S. N. Abraham, Mast cell activators: a new class of highly effective vaccine adjuvants, Nat Med, 14 (2008) 536-541.
[5] B. Amulic, C. Cazalet, G. L. Hayes, K. D. Metzler, A. Zychlinsky, Neutrophil function: from mechanisms to disease, Annu Rev Immunol, 30 (2012) 459-489.
[6] J. Jee, A. Bonnegarde-Bernard, A. Duverger, Y. Iwakura, E. Cormet-Boyaka, T. L. Martin, H. E. Steiner, R. C. Bachman, P. N. Boyaka, Neutrophils negatively regulate induction of mucosal IgA responses after sublingual immunization, Mucosal Immunol, 8 (2015) 735-745.
[7] N. Borregaard, O. E. Sorensen, K. Theilgaard-Monch, Neutrophil granules: a library of innate immunity proteins, Trends Immunol, 28 (2007) 340-345.
[8] T. G. Liou, E. J. Campbell, Nonisotropic enzyme-inhibitor interactions: a novel nonoxidative mechanism for quantum proteolysis by human neutrophils, Biochemistry, 34 (1995) 16171-16177.
[9] E. P. Reeves, H. Lu, H. L. Jacobs, C. G. Messina, S. Bolsover, G. Gabella, E. O. Potma, A. Warley, J. Roes, A. W. Segal, Killing activity of neutrophils is mediated through activation of proteases by K+flux, Nature, 416 (2002) 291-297.
[10] F. Dallegri, L. Ottonello, Tissue injury in neutrophilic inflammation, Inflamm Res, 46 (1997) 382-391.
[11] Y. Morohoshi, K. Matsuoka, H. Chinen, N. Kamada, T. Sato, T. Hisamatsu, S. Okamoto, N. Inoue, H. Takaishi, H. Ogata, Y. Iwao, T. Hibi, Inhibition of neutrophil elastase prevents the development of murine dextran sulfate sodium-induced colitis, J Gastroenterol, 41 (2006) 318-324.
[12] J. A. Voynow, B. M. Fischer, D. E. Malarkey, L. H. Burch, T. Wong, M. Longphre, S. B. Ho, W. M. Foster, Neutrophil elastase induces mucus cell metaplasia in mouse lung, Am J Physiol Lung Cell Mol Physiol, 287 (2004) L1293-1302.
[13] R. Benabid, J. Wartelle, L. Malleret, N. Guyot, S. Gangloff, F. Lebargy, A. Belaaouaj, Neutrophil elastase modulates cytokine expression: contribution to host defense against Pseudomonas aeruginosa-induced pneumonia, J Biol Chem, 287 (2012) 34883-34894.
[14] A. Chawla, G. Alatrash, A. V. Philips, N. Qiao, P. Sukhumalchandra, C. Kerros, I. Diaconu, V. Gall, S. Neal, H. L. Peters, K. Clise-Dwyer, J. J. Molldrem, E. A. Mittendorf, Neutrophil elastase enhances antigen presentation by upregulating human leukocyte antigen class I expression on tumor cells, Cancer Immunol Immunother, 65 (2016) 741-751.
[15] K. Kawabata, M. Suzuki, M. Sugitani, K. Imaki, M. Toda, T. Miyamoto, ONO-5046, a novel inhibitor of human neutrophil elastase, Biochem Biophys Res Commun, 177 (1991) 814-820.
[16] C. W. Yang, B. S. Strong, M. J. Miller, E. R. Unanue, Neutrophils influence the level of antigen presentation during the immune response to protein antigens in adjuvants, J Immunol, 185 (2010) 2927-2934.
[17] J. Y. Huang, Q. L. Zhou, C. H. Huang, Y. Song, A. G. Sharma, Z. Liao, K. Zhu, M. W. Massidda, R. R. Jamieson, J. Y. Zhang, D. G. Tenen, Z. Y. Jiang, Neutrophil Elastase Regulates Emergency Myelopoiesis Preceding Systemic Inflammation in Diet-induced Obesity, J Biol Chem, 292 (2017) 4770-4776.
[18] U. Meyer-Hoffert, O. Wiedow, Neutrophil serine proteases: mediators of innate immune responses, Curr Opin Hematol, 18 (2011) 19-24.
[19] K. A. Fairfax, M. P. Gantier, F. Mackay, B. R. Williams, C. E. McCoy, IL-10 regulates Aicda expression through miR-155, J Leukoc Biol, 97 (2015) 71-78.
[20] O. Pabst, New concepts in the generation and functions of IgA, Nat Rev Immunol, 12 (2012) 821-832.
[21] A. Roghanian, E. M. Drost, W. MacNee, S. E. Howie, J. M. Sallenave, Inflammatory lung secretions inhibit dendritic cell maturation and function via neutrophil elastase, Am J Respir Crit Care Med, 174 (2006) 1189-1198.
[22] A. Biro, G. Sarmay, Z. Rozsnyay, E. Klein, J. Gergely, A trypsin-like serine protease activity on activated human B cells and various B cell lines, Eur J Immunol, 22 (1992) 2547-2553.
[23] J. W. Lillard, Jr., P. N. Boyaka, O. Chertov, J. J. Oppenheim, J. R. McGhee, Mechanisms for induction of acquired host immunity by neutrophil peptide defensins, Proc Natl Acad Sci U S A, 96 (1999) 651-656.
[24] A. Duverger, J. M. Cane, J. Jee, S. H. Leppla, E. Cormet-Boyaka, W. J. Tang, D. Tome, P. N. Boyaka, Contributions of edema factor and protective antigen to the induction of protective immunity by Bacillus anthracis edema toxin as an intranasal adjuvant, J Immunol, 185 (2010) 5943-5952.
[25] A. Duverger, R. J. Jackson, F. W. van Ginkel, R. Fischer, A. Tafaro, S. H. Leppla, K. Fujihashi, H. Kiyono, J. R. McGhee, P. N. Boyaka, Bacillus anthracis edema toxin acts as an adjuvant for mucosal immune responses to nasally administered vaccine antigens, J Immunol, 176 (2006) 1776-1783.

### Example 7

### A Novel Supplementation Approach to Enhance Host Response to Sublingual Vaccination

Sublingual immunization is emerging as an alternative to nasal immunization and induction of mucosal IgA responses. Using Bacillus anthracis edema toxin (EdTx) as an adjuvant, it was previously showed that innate responses triggered after sublingual immunization could limit generation of IgA responses. Co-administration of a neutrophil elastase inhibitor (NEI) was tested whether it could rescue the ability of EdTx to induce broad antibody responses, including mucosal IgA. NEI supplementation of sublingual vaccines containing EdTx promoted antigen-specific serum IgA responses but also enhanced serum IgG1, and IgG2b responses. This enhancing effect of NEI did not extend to all antibody isotypes and IgG subclasses, since it reduced serum IgE responses and did not affect IgG2a/c and IgG3 responses. The NEI also promoted anti-Bacillus anthracis protective antigen (PA) neutralizing antibodies and enhanced high affinity IgG1 and IgA antibodies. In addition to serum IgA, NEI supplementation stimulated antigen-specific mucosal IgA responses in the GI tract, and enhanced antigen-specific IgG responses in vaginal washes. Analysis of CD4⁺ T helper cell responses revealed that co-administration of NEI broadened the profile of cytokine responses, by stimulating Th1, Th2, Th17, and Tfh cytokines. It was also noted that NEI had a higher stimulatory effect on IL-5, IL-10, IL-17 responses.

### Background

Needle-free vaccines delivered via mucosal surface have the potential of being better-accepted by the most vulnerable and commonly vaccinated population of children. They also present higher likelihood to generate the necessary B and T cell responses for optimal protection at the portal of entry of infectious agents, in addition to promoting the levels of systemic immunity generally achieved by conventional injected vaccines (1). Secretory IgA (SIgA) represents the hallmark of immune responses at mucosal surfaces. The high resistance of these polymeric immunoglobulins to degradation in the harsh environment of mucosal surfaces, including the lumen of the gastrointestinal tract, allow them to provide frontline protection at the portal of entry of most infectious microbes (2). While mucosal delivered subunit-vaccines have the potential of stimulating broad mucosal and systemic immune responses, their ability to trigger mucosal IgA relies on the addition of effective vaccine adjuvants. Stimulation of inductive site immune responses in different mucosal sites (i.e., gastrointestinal tract, respiratory tract, rectal) imprints the expression of discrete mucosal homing receptors and addressing which allow effector B and T cells to home in distinct mucosal tissues. For example, intranasal delivery of vaccines containing appropriate mucosal adjuvants can promote specific immune responses in the airways. However, safety issues were reported following intranasal application of ganglioside-binding toxin as adjuvant, and sublingual immunization is now being considered as an alternative to the intranasal route of vaccination. Nonetheless, a major challenge for the development of sublingual vaccine is the identification of appropriate antigen-adjuvant formulations (2, 3).

It was shown that Bacillus anthraces edema toxin (EdTx) is an effective adjuvant capable of promoting both systemic immunity and mucosal SIgA responses against nasally co-administered vaccine antigens (4, 5). However, when EdTx was tested as adjuvant for sublingual vaccination, it promoted antigen-specific IgG responses in the bloodstream but failed to elicit IgA responses in the serum or mucosal secretions (6). This lack of IgA responses was not due to the route of immunization itself, since sublingual immunization could induce these responses when vaccines were administered with a range of adjuvants including bacterial enterotoxins, toll-like receptor ligands, and STING ligands (7-11). Interestingly, the lack of IgA response correlated with the recruitment of neutrophil after sublingual administration of EdTx, and partial depletion of neutrophils before sublingual immunization restored the adjuvant activity of EdTx for IgA responses (6). Neutrophils represent the largest population of myeloid cells in the bloodstream and characterize the initial response to inflammatory events through their own degranulation and cytokine production (12). Unfortunately, depletion of neutrophils prior to immunization is not a feasible approach and thus, new strategies are needed to improve the efficacy of EdTx-based, and possibly other, sublingual vaccines.

Neutrophils are recruited by inflammatory cytokines, including IL-6, IL-β, and TNFα, and were more recently recognized to contribute to the chemotaxis of other myeloid cells through the products released after neutrophil degranulation (12, 13). Neutrophil elastase inhibitors (NEI) are a class of serine protease inhibitors that target the neutrophil granule protein elastase, commonly implicated in chronic lung inflammation (14-16).

### Results

### Co-Sdministration of a NEI Enhances Serum IgG1 and IgG2b Responses to a Sublingual Vaccine

It was first tested whether supplementation with a NEI affects IgG responses induced by a model sublingual vaccine containing EdTx as adjuvant. Analysis of OVA-specific IgG1 responses revealed that NEI increased the magnitude of responses induced by EdTx, and this effect was evident as early as day 14 after the first immunization (FIG. 33A). It was also found that the NEI used in these studies had an intrinsic adjuvant activity and increased IgG1 responses when co-administered with antigen in the absence of EdTx (FIG. 33A). Analysis of other IgG subclasses showed no evidence that the NEI enhanced IgG2a/c, or IgG3 when co-administered with antigen in the absence of EdTx (FIGS. 33B and 33C). However, the NEI significantly increased serum IgG2b responses induced by EdTx as adjuvant (FIGS. 33B and 33C).

Supplementation with a NEI Suppresses Serum IgE Responses Induced by a Sublingual Vaccine

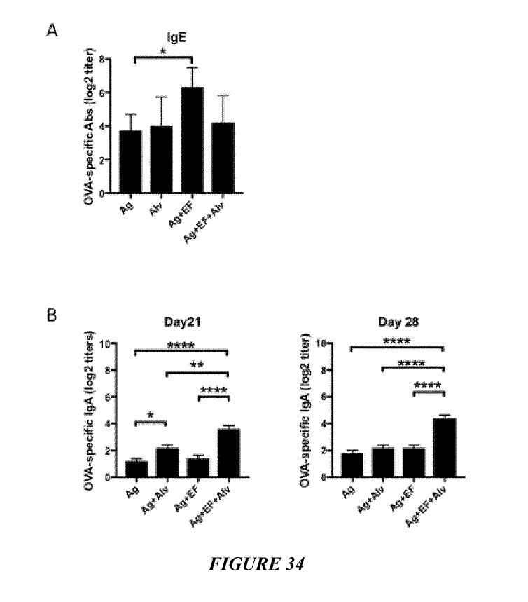

Since the presence of NEI differentially regulated IgG subclass responses, the potential effects on other immunoglobulin isotype responses was addressed. Sublingual immunization with EdTx as adjuvant induced OVA-specific IgE responses which peaked 2 weeks after the first immunization (day 14) (FIG. 34A). Co-administration of NEI significantly reduced the magnitude of IgE responses to levels barely at the detection threshold (FIG. 34A). Furthermore, no IgE response was detected following sublingual application of NEI and antigen alone (FIG. 34A).

Supplementation of a Sublingual Vaccine with a NEI Promotes Serum IgA Responses

As previously reported (6), sublingual immunization with EdTx as adjuvant failed to induce OVA-specific serum IgA responses (FIG. 34B). Co-administration of antigen and NEI alone did not result in the development of serum IgA responses. Interestingly, the NEI promoted serum IgA responses in mice immunized with EdTx as adjuvant (FIG. 34B).

NEI Supplementation Enhances the Amount of High Affinity Abs and Toxin Neutralizing Responses in Mice Immunized with EdTx as Adjuvant

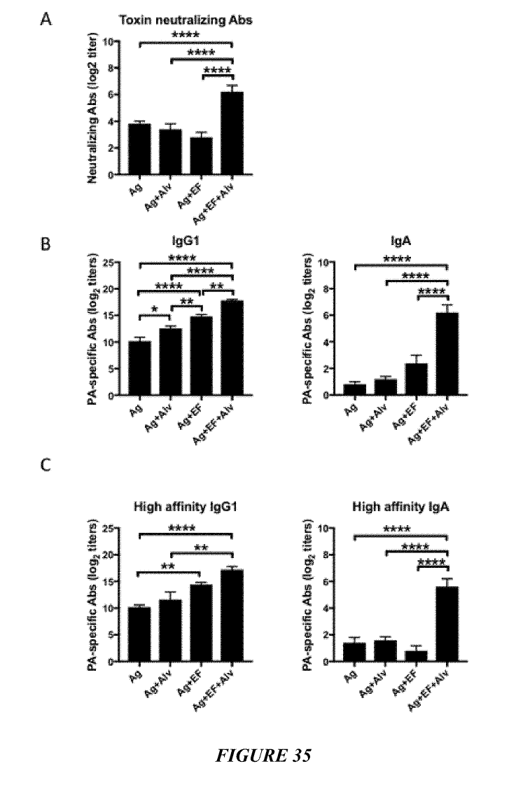

Results summarized above show that NEI supplementation improves the kinetic of IgG1 responses and allows mice immunized with EdTx to reach maximum IgG1 titers more quickly (FIG. 33). They also show that NEI broadens the antibody responses by enhancing IgG2b and promoting IgA (FIGS. 33 and 34). To determine the biological significance of the broader antibody responses measured in the blood of mice administered vaccines supplemented with NEI, anti-PA neutralizing antibodies are assessed. Serum of mice immunized with antigen alone (OVA plus PA), antigen plus NEI, or antigen plus NEI contained low and comparable levels of anti-PA neutralizing antibodies (FIG. 35A). On the other hand, anti-PA neutralizing antibody titers were significantly increased in the group of mice that received EdTx and NEI (FIG. 35A).

It was also investigated whether the enhanced neutralization of LeTx toxicity was due to the increase in anti-PA antibody titers or whether it also resulted from an increase in high affinity antibodies. As with anti-OVA responses above (FIG. 33), NEI supplementation enhanced the titers of PA-specific IgG1 and IgA responses in mice with EdTx as adjuvant (FIG. 35B). Most importantly, the IgG1 and IgA promoted by NEI supplementation were high affinity antibodies (FIG. 35C).

Supplementation of a Sublingual Vaccine with a NEI Promotes Mucosal IgA Responses

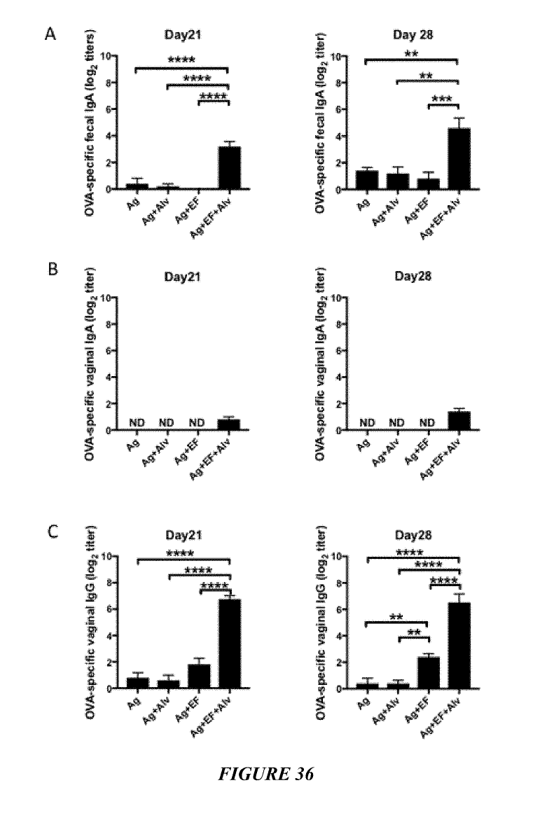

Our previous studies have shown that a major limitation of EdTx as adjuvant for sublingual vaccines is its inability to promote IgA responses in the bloodstream and in mucosal secretions (6). Sublingual application of antigen alone or antigen plus NEI did not stimulate production of antigen-specific IgA in gut. Addition of NEI to a vaccine formulation containing antigen and EdTx resulted in induction of fecal IgA responses, which were detectable 3 weeks after the first immunization (day 21) and were further increased one week later (day 28) (FIG. 36A). To determine if NEI supplementation promoted generalized IgA responses in other mucosal secretion, these responses were analyzed in vaginal washes. Unlike mucosal secretions of the GI tract, the vaginal washes of mice immunized with EdTx and NEI showed only non-significant levels of antigen-specific IgA responses (FIG. 36B). However, NEI supplementation was able to enhance the vaginal IgG responses induced by EdTx as adjuvant (FIG. 36C). These data show that NEI might promote expression of gut homing receptors by IgA producing cells, while the elevated IgG antibodies found in the secretions of the genitourinary tract might be coming from diffusion of IgG from the blood.

### Addition of NEI to a Sublingual Vaccine Broadens the Profile of CD4+T Helper Cell Responses

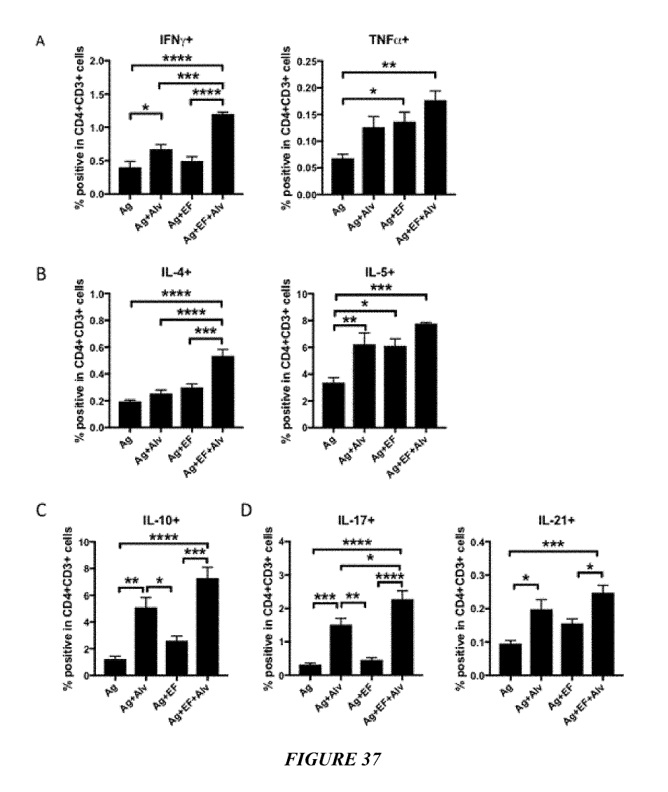

Flow cytometry analysis of cytokines produced by CD4+ T cells were analyzed upon in vitro restimulation with OVA to elucidate the profile of T helper cytokines that supported the antibody responses induced by addition of NEI. Spleen cells from mice immunized with antigens plus NEI exhibited the same frequencies of Th1 cells (IFNγ^{|}CD4^{|}T cells and TNFα^{|}CD4^{|} T cells) as spleen cells from mice that received antigen plus EdTx (FIG. 37A). The frequencies of Th2 cells producing IL-5 were also similar between these two groups, but mice immunized with antigens plus NEI exhibited lower frequencies of IL-4⁺ CD4⁺ cells (FIG. 37B). These analyses also show that immunization with antigen plus NEI promoted higher frequencies of IL-10+ CD4⁺ T cells and IL-17⁺ CD4⁺ T cells than immunization with antigen plus EdTx (FIGS. 37C and 37D). Furthermore, NEI and EdTx promoted the same frequencies of antigen-specific IL-21⁺ CD4 T cells (FIG. 37D). Consistent with the broad increase in antibody responses, co-administration of EdTx and NEI significantly increased all CD4⁺ T helper cytokine responses, including Th1, Th2, Th17 and Tfh responses (FIG. 37).

### Discussion

The sublingual route is currently used for drug delivery and allergen specific immunotherapy (ASIT). Sublingual vaccination is theoretically very simple as it involves application of liquid or rapidly dissolving tablets to the floor of the oral cavity. Like vaccines delivery at other mucosal sites, sublingual vaccines have the potential of producing mucosal SIgA in addition to IgG responses in the general bloodstream. Because sublingually administered vaccines are less likely to reach CNS tissues, they are potentially safer than nasal vaccines. Despite these advantages, identification of the appropriate adjuvant formulations has limited the development of sublingual vaccines. Here, it was shown that supplementation of the EdTx adjuvant with a chemical inhibitor of a neutrophil function broadens B and T cell responses induced after sublingual immunization and promotes antigen-specific SIgA Abs.

It was shown that the breadth of antibody responses induced by EdTx as a sublingual adjuvant is limited by the recruitment of neutrophils in mucosal inductive sites (6). While this work was the first to establish an inverse relationship between the presence of neutrophils and production of IgA, the mechanisms underlying the suppressive effect of neutrophils on IgA production remained elusive. It was previously shown that neutrophil peptide defensins enhance IgA, but not IgA against nasally co-administered vaccine antigen (17). The primary (or azurophilic) granules of neutrophils contain defensins, myeloperoxidase, lysozymes, and three serine proteases: neutrophil elastase, cathepsin G and protease 3 (12, 18). It is now shown that inhibition of a single function of neutrophils can rescue the adjuvant activity of EdTx and promote IgA responses both in the blood stream and in mucosal tissues. This finding is significant because the use of a serine protease inhibitor might be a more realistic alternative to neutrophil depletion as a strategy for promoting IgA responses. It also raises the question of the mechanism behind neutrophil elastase limiting IgA production. Neutrophil elastase and the related serine proteases cathepsin G and proteinase 3, were shown to regulate cytokine responses through activation or degradation of cytokines, cytokine receptors, or toll-like receptor (12, 19). Separate studies have shown that NEI induce the expression of IL-10 mRNA but also AID and BAFF responses by spleen cells in vitro (Attia 2018, submitted). Because IL-10 facilitates Ig class switching for production of IgA (20) and regulate the expression of AID for induction of Ig CSR (21), this may be a mechanism for enhanced IgA responses in mice sublingually immunized with EdTx in the presence of NEI.

Sublingual immunization of mice with STING ligands as adjuvant induces IgA responses in mucosal tissues of the airways (7, 10) and the genitourinary tract (10). However, neither CpG nor the STING ligand 3′33-cGAMP as sublingual adjuvants induced SIgA in the secretions of the GI tract (i.e., in fecal extracts) (10). As shown here, when added to an EdTx-based sublingual vaccine, NEI primarily promoted SIgA responses in mucosal secretions of the GI tract. No significant levels of IgA were measured in other mucosal secretions although IgG responses were significantly increased in vaginal washes. The best-described factors that regulate homing of immune cells into the gut are CCR9 and gut homing receptors α4β7 (22, 23). These factors are induced through signals provided by retinoid acid, a product of myeloid cells in mucosal tissues (22). No study has yet examined whether neutrophils regulate expression of gut homing receptors. But it is worth noting that in conditions of normal homeostasis, neutrophils are not present in mucosal tissues where the concentration of IgA is the highest in the body. The observation that NEI primarily promotes SIgA in the gut shows that it either directly stimulated retinoic acid production or stimulated cytokines such as IL-5, IL-6 and IL-21, which promote or enhance expression of gut homing receptors (24, 25).

IgA was not the only immunoglobulin isotype affected by supplementation of the EdTx-based sublingual vaccine. In fact, NEI also enhanced IgG1 and IgG2b responses. This is consistent with the previous report that elastase inhibits the maturation and function of dendritic cells, including expression of the costimulatory molecules CD40, CD80 and CD86 (26). Thus, the presence of NEI may have promoted the maturation of dendritic cells and their expression BAFF and APRIL and perhaps costimulatory molecules (i.e., CD40) for enhanced antibody production. Another important point revealed in the present study is that the quality of antibody responses was also improved by the presence of NEI. Indeed, NEI increased the titers of high affinity IgG and IgA antibodies, an effect that translated into higher titers of anti-PA neutralizing antibodies. These findings are in line with the recent report that neutrophil regulate germinal center formation in secondary lymphoid organs and production of autoreactive plasma cells in lupus (27). Analysis of antigen-specific T helper cytokine responses provided some clues on how NEI improved the breath, magnitude and quality of antibody responses in mice immunized via the sublingual route with EdTx as adjuvant. It was found that addition of NEI resulted in higher frequencies of Th1 cells producing IFN-γ, Th2 cells producing IL-5 and IL-10, Th17 cells producing IL-17, and Tfh cells producing IL-21. This broad profile of Th cell cytokine responses has most likely contributed to an increased B cell survival via signals from IL-5 and IL-17 (28-32). It is also consistent with the observation by other that neutrophils preferentially accumulate in proximity of T cells in secondary lymphoid organs during the early stage of lupus and that neutrophil depletion accelerate germinal center formation (27). In this study, the NEI may have played the same role as neutrophil depletion and enhanced development of germinal centers and production of high affinity antibodies by facilitating differentiation of Tfh producing IL-21.

Altogether, these results show that supplementation of sublingual vaccine by co-administration of a NEI can improve the kinetic, breadth and the quality of antibody responses both in the bloodstream and in mucosal tissues. It is also shown that NEI alone exhibited some adjuvant activity and enhanced IgG1 and IgA responses against the sublingually co-administered vaccine antigens.

Material and methods

Animals

Female C57BL/6J mice were obtained from The Jackson Laboratory (Bar Harbor, Me.). Mice were maintained in a specific pathogen-free environment and used at 10±2 wks of age. All the experiments were approved by and performed in accordance with the guidelines of NIH and The Ohio State University IACUC.

Sublingual immunization and samples collection

Sublingual immunization was performed as previously described (6, 10) by administration of 10-13 μl of a PBS solution to anesthetized mice. All mice received antigen [i.e., 20 μg of PA (Bacillus anthracis protective antigen, BEI Resources, Manassas, Va.) plus 40 μg of ovalbumin grade V (Sigma-Aldrich, Saint Louis, Mo.)]. To assess the adjuvant activity of Bacillus anthracis edema toxin [which results from combination of PA and Bacillus anthracis edema factor (EF), groups of mice were given antigens plus 15 μg of EF (BEI Biosource)]. To address the effect of NEI supplementation, groups of mice given antigens plus 100 μM of Alvelestat (AZD9668, C₂₅H₂₂F₃N₅O₄S) (Selleckchem, Houston, Tex.), or antigen plus EF plus Alvelestat. All groups were immunized at weekly intervals for 3 consecutive weeks (days 0, 7, and 14). Serum, fecal pellet and vaginal wash samples were collected weekly, and nasal washes were collected at day 28.

Evaluation of Antigen-Specific Antibody Responses

To determine OVA-specific and PA-specific antibody titers, ELISA was performed with OVA-coated plates as described previously (4-6, 33, 34) Briefly, microtiter plates were coated with OVA (1 mg/ml) or PA (15 μg/ml). For detection of OVA-specific IgG and IgA Abs, serial dilutions of serum or fecal material extract were added to the plates and the binding antibodies were detected with HRP-conjugated anti-mouse γ- or α-heavy chain-specific antisera (Southern Biotech Associates Inc., Birmingham, Ala.). Biotin-conjugated rat anti-mouse IgG1, IgG2a/c, IgG2b or IgG3 monoclonal Abs and HRP-conjugated streptavidin (BD Bioscience, san Jose, Calif.) were used to measure IgG subclass responses. The Ab titers were determined as the last dilutions of samples with an absorbance of >0.1 above that of control samples from naïve mice.

For assessment of IgA levels in the intestinal secretions, freshly emitted fecal pellets were normalized by homogenization in PBS (1 ml per 0.1 g feces). After centrifugation, dilutions of supernatants were used for evaluation of antigen-specific IgA levels as described above.

Quantification of High Affinity Antibody Responses

High affinity antibody responses were measured by ELISA as described above with a minor modification. Briefly, plates were coated with PA and incubated with dilutions of the samples. Urea (4 mM) was then added to the plates to remove antibodies that bound to the antigen with low affinity. After washing, the detection antibodies were added and the remaining steps of the ELISA conducted as described above.

Assessment of Toxin Neutralizing Antibodies

Toxin neutralization assay was performed as previously described (4-6). Briefly, sample dilutions were added to cultures of J774 macrophages. Bacillus anthracis lethal toxin (LeTx) [i.e., PA plus Bacillus anthracis lethal factor (LF, List Biological, Campbell, Calif.)] were then added to the plates. The viability of macrophages was assessed after overnight incubation and the toxin neutralizing antibody titers were determined as the lowest concentration of serum that protects macrophages from the cytotoxicity of LeTx.

Analysis of Antigen-Specific T Helper Cell Cytokines

Antigen-specific T helper cell cytokine responses were analyzed by flow cytometry after in vitro restimulation and intracellular staining with cytokine-specific fluorescent antibodies. Briefly, splenocytes and cervical lymph nodes were collected on day 28 after the first immunization and restimulated with antigen (i.e., OVA) in vitro as previously described (4-6). After 5 days culture, cells were subjected to extracellular staining with lineage-specific antibodies [i.e., anti-CD3, and anti-CD4 (Biolegend, San Diego, Calif.)] and, after fixation, to intracellular staining with Th1, Th2, Th17, and Tfh cytokine-specific antibodies (Biolegend). Labeled cells were analyzed with an Attune NxT flow cytometer (Thermo Fisher Scientific, Waltham, Mass.).

Statistical Analysis

Results are expressed as the mean±1 one standard deviation. Statistical significance was determined by one-way ANOVA, followed by Tukey post-hoc test. All statistical analyses were performed with the StataSE 12.0 software (StataCorp LLC, College Station, Tex.) and Prism 7 software (Graphpad Software, La Jolla, Calif.).

References Cited in this example
1. Holmgren, J., and C. Czerkinsky. 2005. Mucosal immunity and vaccines. Nat Med 11: S45-53.
2. Boyaka, P. N. 2017. Inducing Mucosal IgA: A Challenge for Vaccine Adjuvants and Delivery Systems. J lmmunol 199: 9-16.
3. Czerkinsky, C., and J. Holmgren. 2010. Topical immunization strategies. Mucosal Immunol 3: 545-555.
4. Duverger, A., J. M. Carre, J. Jee, S. H. Leppla, E. Cormet-Boyaka, W. J. Tang, D. Tome, and P. N. Boyaka. 2010. Contributions of edema factor and protective antigen to the induction of protective immunity by Bacillus anthracis edema toxin as an intranasal adjuvant. J lmmunol 185: 5943-5952.
5. Duverger, A., R. J. Jackson, F. W. van Ginkel, R. Fischer, A. Tafaro, S. H. Leppla, K. Fujihashi, H. Kiyono, J. R. McGhee, and P. N. Boyaka. 2006. Bacillus anthracis edema toxin acts as an adjuvant for mucosal immune responses to nasally administered vaccine antigens. J lmmunol 176: 1776-1783.
6. Jee, J., A. Bonnegarde-Bernard, A. Duverger, Y. Iwakura, E. Cormet-Boyaka, T. L. Martin, H. E. Steiner, R. C. Bachman, and P. N. Boyaka. 2015. Neutrophils negatively regulate induction of mucosal IgA responses after sublingual immunization. Mucosal Immunol 8: 735-745.
7. Ebensen, T., J. Debarry, G. K. Pedersen, P. Blazejewska, S. Weissmann, K. Schulze, K. C. McCullough, R. J. Cox, and C. A. Guzman. 2017. Mucosal Administration of Cycle-Di-Nucleotide-Adjuvanted Virosomes Efficiently Induces Protection against Influenza H5N1 in Mice. Front Immunol 8: 1223.
8. Huang, C. F., T. C. Wu, C. C. Wu, C. C. Lee, W. T. Lo, K. S. Hwang, M. L. Hsu, and H. J. Peng. 2011. Sublingual vaccination with sonicated Salmonella proteins and mucosal adjuvant induces mucosal and systemic immunity and protects mice from lethal enteritis. APMIS 119: 468-478.
9. Kraan, H., H. Vrieling, C. Czerkinsky, W. Jiskoot, G. Kersten, and J. P. Amorij. 2014. Buccal and sublingual vaccine delivery. J Control Release 190: 580-592.
10. Martin, T. L., J. Jee, E. Kim, H. E. Steiner, E. Cormet-Boyaka, and P. N. Boyaka. 2017. Sublingual targeting of STING with 3′3′-cGAMP promotes systemic and mucosal immunity against anthrax toxins. Vaccine 35: 2511-2519.
11. Song, J. H., H. H. Nguyen, N. Cuburu, T. Horimoto, S. Y. Ko, S. H. Park, C. Czerkinsky, and M. N. Kweon. 2008. Sublingual vaccination with influenza virus protects mice against lethal viral infection. Proc Natl Acad Sci USA 105: 1644-1649.
12. Amulic, B., C. Cazalet, G. L. Hayes, K. D. Metzler, and A. Zychlinsky. 2012. Neutrophil function: from mechanisms to disease. Annu Rev Immunol 30: 459-489.
13. Soehnlein, O., L. Lindbom, and C. Weber. 2009. Mechanisms underlying neutrophil-mediated monocyte recruitment. Blood 114: 4613-4623.
14. Doring, G. 1994. The role of neutrophil elastase in chronic inflammation. Am J Respir Crit Care Med 150: S114-117.
15. Henriksen, P. A. 2014. The potential of neutrophil elastase inhibitors as anti-inflammatory therapies. Curr Opin Hematol 21: 23-28.
16. Stevens, T., K. Ekholm, M. Granse, M. Lindahl, V. Kozma, C. Jungar, T. Ottosson, H. Falk-Hakansson, A. Churg, J. L. Wright, H. Lal, and A. Sanfridson. 2011. AZD9668: pharmacological characterization of a novel oral inhibitor of neutrophil elastase. Pharmacol Exp Ther 339: 313-320.
17. Lillard, J. W., Jr., P. N. Boyaka, O. Chertov, J. J. Oppenheim, and J. R. McGhee. 1999. Mechanisms for induction of acquired host immunity by neutrophil peptide defensins. Proc Natl Acad Sci USA 96: 651-656.
18. Borregaard, N., O. E. Sorensen, and K. Theilgaard-Monch. 2007. Neutrophil granules: a library of innate immunity proteins. Trends Immunol 28: 340-345.
19. Meyer-Hoffert, U., and O. Wiedow. 2011. Neutrophil serine proteases: mediators of innate immune responses. Curr Opin Hematol 18: 19-24.
20. Pabst, O. 2012. New concepts in the generation and functions of IgA. Nat Rev Immunol 12: 821-832.
21. Fairfax, K. A., M. P. Gantier, F. Mackay, B. R. Williams, and C. E. McCoy. 2015. IL-10 regulates Aicda expression through miR-155. J Leukoc Biol 97: 71-78.
22. Hammerschmidt, S. I., M. Friedrichsen, J. Boelter, M. Lyszkiewicz, E. Kremmer, O. Pabst, and R. Forster. 2011. Retinoic acid induces homing of protective T and B cells to the gut after subcutaneous immunization in mice. J Clin Invest 121: 3051-3061.
23. Mora, J. R., and U. H. von Andrian. 2008. Differentiation and homing of IgA-secreting cells. Mucosal Immunol 1: 96-109.
24. Cao, A. T., S. Yao, B. Gong, R. I. Nurieva, C. O. Elson, and Y. Cong. 2015. Interleukin (IL)-21 promotes intestinal IgA response to microbiota. Mucosal Immunol 8: 1072-1082.
25. Mora, J. R., M. Iwata, B. Eksteen, S. Y. Song, T. Junt, B. Senman, K. L. Otipoby, A. Yokota, H. Takeuchi, P. Ricciardi-Castagnoli, K. Rajewsky, D. H. Adams, and U. H. von Andrian. 2006. Generation of gut-homing IgA-secreting B cells by intestinal dendritic cells. Science 314: 1157-1160.
26. Roghanian, A., E. M. Drost, W. MacNee, S. E. Howie, and J. M. Sallenave. 2006. Inflammatory lung secretions inhibit dendritic cell maturation and function via neutrophil elastase. Am J Respir Crit Care Med 174: 1189-1198.
27. Bird, A. K., M. Chang, J. Barnard, B. I. Goldman, N. Meednu, J. Rangel-Moreno, and J. H. Anolik. 2017. Neutrophils Slow Disease Progression in Murine Lupus via Modulation of Autoreactive Germinal Centers. J lmmunol 199: 458-466.
28. Blaho, V. A., M. W. Buczynski, E. A. Dennis, and C. R. Brown. 2009. Cyclooxygenase-1 orchestrates germinal center formation and antibody class-switch via regulation of IL-17. J lmmunol 183: 5644-5653.
29. Hirota, K., J. E. Turner, M. Villa, J. H. Duarte, J. Demengeot, O. M. Steinmetz, and B. Stockinger. 2013. Plasticity of Th17 cells in Peyer's patches is responsible for the induction of T cell-dependent IgA responses. Nat Immunol 14: 372-379.
30. Horikawa, K., and K. Takatsu. 2006. Interleukin-5 regulates genes involved in B-cell terminal maturation. Immunology 118: 497-508.
31. Mizoguchi, C., S. Uehara, S. Akira, and K. Takatsu. 1999. IL-5 induces IgG1 isotype switch recombination in mouse CD38-activated sIgD-positive B lymphocytes. J lmmunol 162: 2812-2819.
32. Sonoda, E., R. Matsumoto, Y. Hitoshi, T. Ishii, M. Sugimoto, S. Araki, A. Tominaga, N. Yamaguchi, and K. Takatsu. 1989. Transforming growth factor beta induces IgA production and acts additively with interleukin 5 for IgA production. J Exp Med 170: 1415-1420.
33. Bonnegarde-Bernard, A., J. Jee, M. J. Fial, F. Aeffner, E. Cormet-Boyaka, I. C. Davis, M. Lin, D. Tome, M. Karin, Y. Sun, and P. N. Boyaka. 2014. IKKbeta in intestinal epithelial cells regulates allergen-specific IgA and allergic inflammation at distant mucosal sites. Mucosal Immunol 7: 257-267.
34. Bonnegarde-Bernard, A., J. Jee, M. J. Fial, H. Steiner, S. DiBartola, I. C. Davis, E. Cormet-Boyaka, D. Tome, and P. N. Boyaka. 2014. Routes of allergic sensitization and myeloid cell IKKbeta differentially regulate antibody responses and allergic airway inflammation in male and female mice. PLoS One 9: e92307.

### Example 8

### Methods for Induction of High Affinity Antibody and Mucosal Immunity by Parenterally Delivered Vaccines

Most infectious agents enter the body through mucosal tissues. Infectious agents induce T and B cells responses, including mucosal IgA, in mucosal tissues could limit infectivity and provide first line of defense against infectious agents.

Most current vaccines are injected vaccines (parenterally delivered). Many of these vaccines contain alum (aluminum salts) as adjuvant. Current issues with vaccines are that they often require multiple boosts to achieve high serum antibody titers and to induce high affinity antibodies needed for protection. In addition, they do not stimulate expression of mucosal homing receptors by effector T and B cells. In addition, most vaccines do not induce mucosal IgA responses and cytotoxic T cells in mucosal tissues.

Improved vaccines should quickly induce high affinity or functionally protective antibodies in the bloodstream, limit production of IgE (by some adjuvants), induce expression mucosal homing receptors by effector B and T cells to generate two layers of protections against infectious agents (i.e., mucosal immunity+immunity in the bloodstream), and induce mucosal IgA.

Here, the inventors have modified current parenteral vaccines by addition of agents that suppress the function of an enzyme produced by innate immune cells. Co-administration of such agents: 1) speed-up generation of antibody responses in the bloodstream; 2) increase production of high affinity IgG and IgA antibodies; 3) induce expression of mucosal homing receptors by effector B and T cells; and 4) promote generation of mucosal IgA response. The inventors have developed compounds and methods for improving vaccines, including parenteral vaccines, that have improved IgG responses in the bloodstream. Thus, co-administration of elastase inhibitors: 1) broaden serum IgG antibody responses; 2) stimulates expression of mucosal homing receptors (CCR9 and α4β7); and 3) triggers IgA responses, including mucosal IgA.

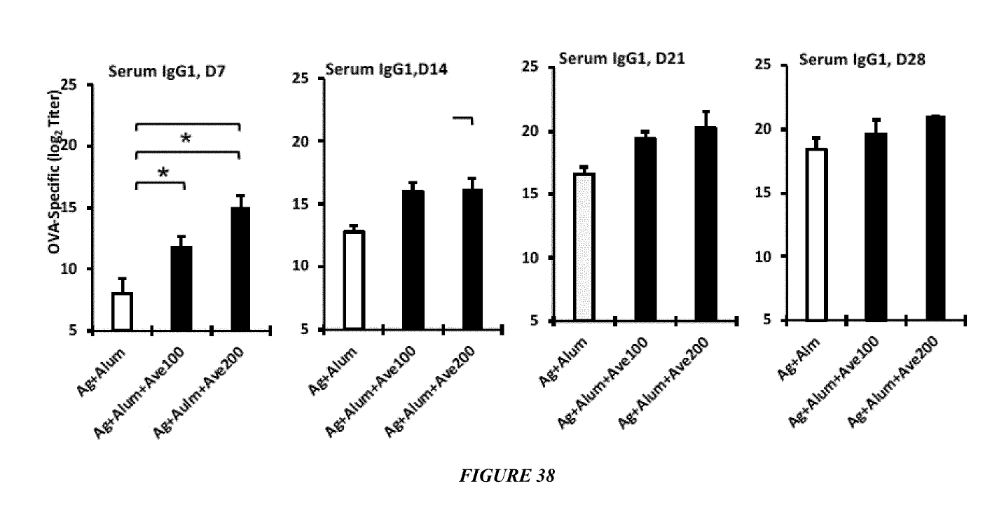

As shown in FIG. 38, co-administration of NEI enhances the kinetic and magnitude of serum IgG in mice immunized parenterally immunized with alum as adjuvant. Mice were immunized three times at weekly intervals by i.p. injection of alum-adsorbed vaccine antigens (40 μg of OVA plus 20 μg of Bacillus anthracis PA) in the absence or presence of the NEI Alvelestat (100 or 200 μM). Blood was collected weekly and titers of OVA-specific serum Ab responses were determined by ELISA. Data are presented as mean antibody titers±one SD and represent one of at least four independent experiments with five mice per group. Thus, supplementation with NEI improves kinetic and magnitude of serum IgG responses to an alum-based parenteral vaccine.

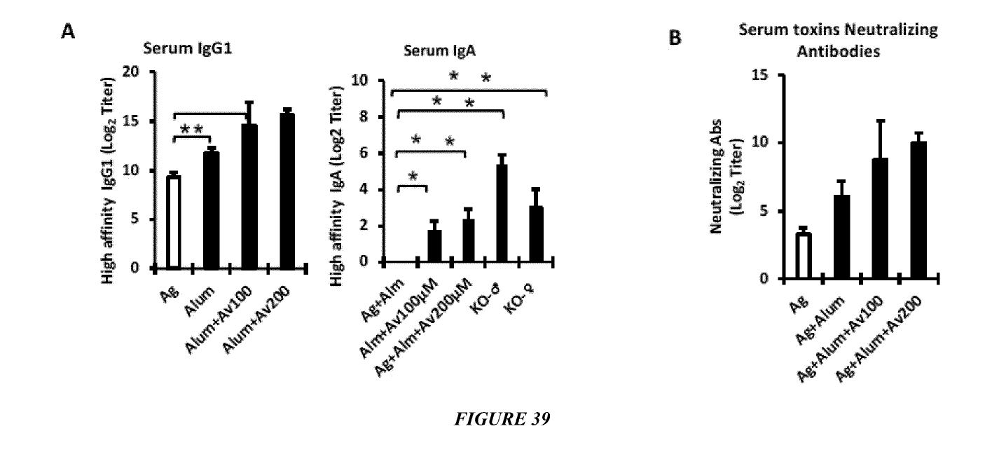

As shown in FIG. 39, co-administration of NEI or ELANE deficiency promotes high affinity Abs with enhanced neutralizing activity. Mice were immunized three times at weekly intervals by i.p. injection of alum-adsorbed vaccine antigens (40 μg of OVA plus 20 μg of Bacillus anthracis PA) alone or in the presence of the NEI Alvelestat (100 or 200 μM). ELANE deficient mice were immunized i.p. injection of alum-adsorbed vaccine antigens (40 μg of OVA plus 20 μg of Bacillus anthracis PA) alone. Serum samples were collected one week after the last immunization (Day 21). (A) High affinity PA-specific serum IgG1 and IgA Ab responses were determined by a modified ELISA where low affinity Abs were removed by incubation with urea (4 mM). (B) Serum anti-PA neutralizing Ab responses were determined by the Bacillus anthracis lethal toxin in vitro macrophage toxicity assay. Thus, supplementation of an alum-based parenteral vaccine with NEI enhances production of high affinity and functionally protective serum antibodies.

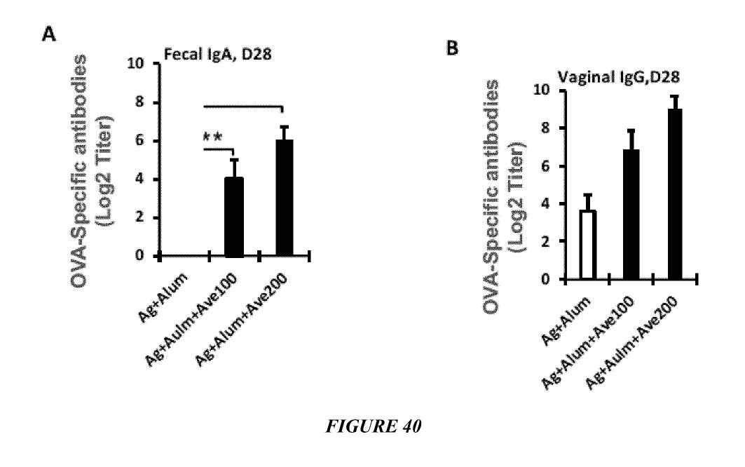

As shown in FIG. 40, co-administration of NEI promotes mucosal IgA responses in the gut of mice immunized parenterally with alum as adjuvant. Mice were immunized three times at weekly intervals by i.p. injection of alum-adsorbed vaccine antigens (40 μg of OVA plus 20 μg of Bacillus anthracis PA) in the absence or presence of the NEI Alvelestat. OVA-specific IgA and IgG were analyzed in fecal extracts (A) and vaginal washes (B) collected two weeks after the last immunization (day 28). Data are presented as mean antibody titers±one SD and represent one of at least four independent experiments with five mice per group. Thus, supplementation of an alum-based parenteral vaccine with NEI mucosal IgA responses in the gut of mice of immunized parenterally with alum as adjuvant.

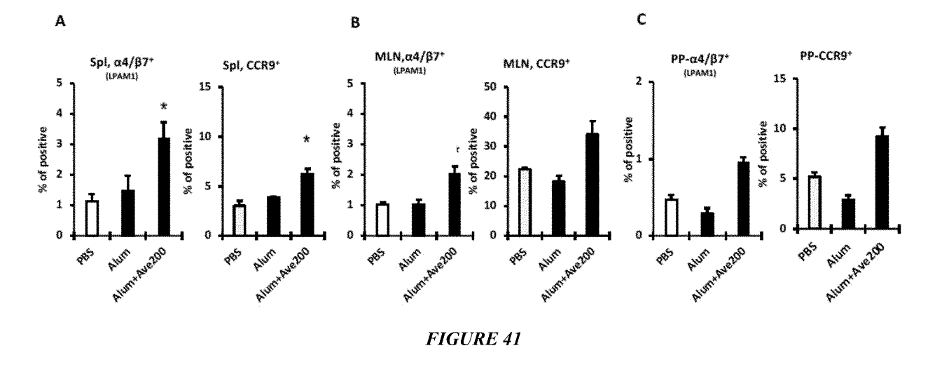

As shown in FIG. 41, co-administration of NEI promotes expression of gut homing receptors in mice immunized parenterally with alum as adjuvant. Mice were given alum-adsorbed vaccine antigens (40 μg of OVA plus 20 μg of Bacillus anthracis PA) alone or in the presence of the NEI Alvelestat (200 μM). Control mice were injected saline (PBS). (A) Spleens, (B) mesenteric lymph nodes, and (C) Peyer's patches were collected 24 hours later and the expression of mucosal homing receptors (i.e., CCR9 and a4b7) by B and T cells was analyzed by flow cytometry.

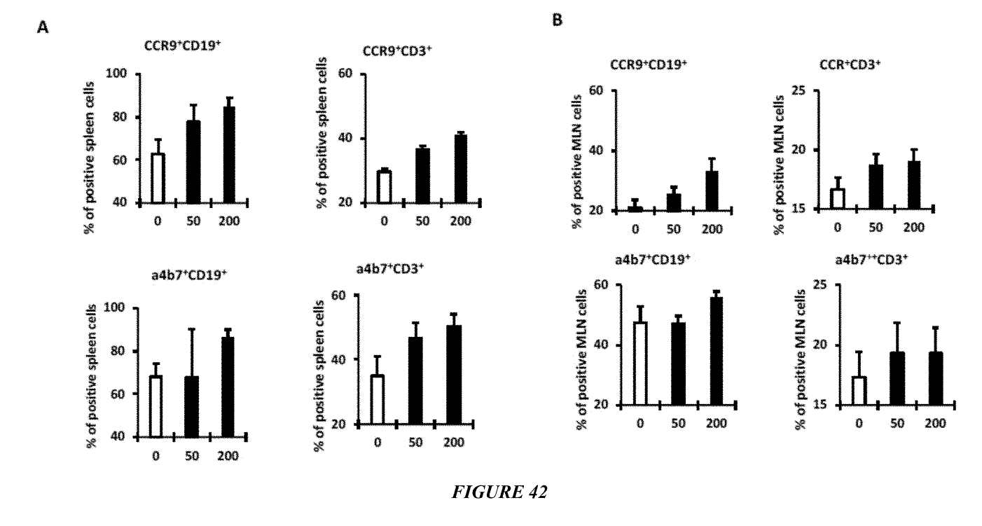

As shown in FIG. 42, NEI promotes expression of gut homing receptors by B and T cells in vitro. Spleen (A) and mesenteric lymph node (B) cells of naïve mice were cultures for 48 hours in the absence or in the presence of the NEI Alvelestat (50 or 200 mM). The expression of mucosal homing receptors CCR9 and a4b7 by B lymphocytes, and T lymphocytes was analyzed by flow cytometry.

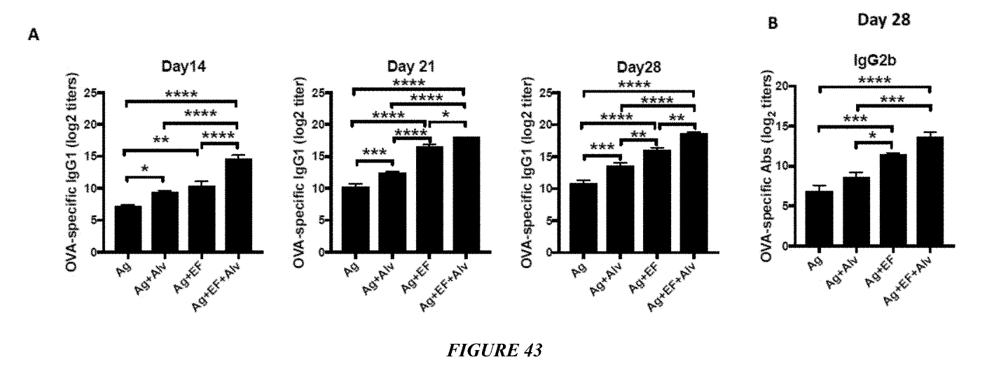

As shown in FIG. 43, sublingual co-application of a NEI enhances antigen-specific serum IgG1 and IgG2b responses. Mice were immunized three times at weekly intervals by sublingual application of antigens (OVA plus PA) alone or antigens plus the NEI Alvelestat. To assess the effect of the NEI on the adjuvanticity of Bacillus anthraces edema toxin [EdTx, which results from combination of PA plus edema factor (EF)], mice were immunized with antigens plus EF or antigens plus EF plus NEI. Serum samples were collected weekly and OVA-specific Ab responses were analyzed by ELISA. (A) Kinetic of IgG1 Ab responses. (B) IgG2b responses two weeks (D28) after the last immunization. Thus, NEI improves the kinetic and magnitude of serum IgG responses to a vaccine given by a non-parenteral route

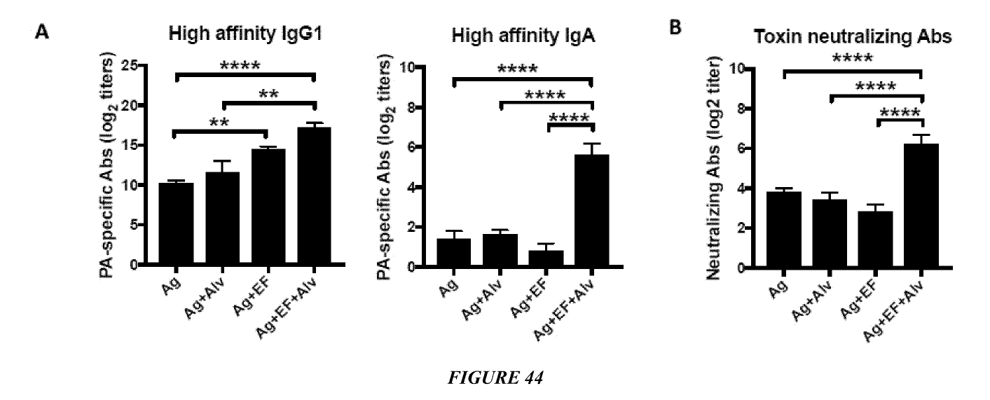

As shown in FIG. 44, sublingual co-application of a NEI promotes high affinity Abs with enhanced neutralizing activity. Mice were immunized three times at weekly intervals by sublingual application of antigens (OVA plus PA) alone, antigens plus the NEI, antigens plus EF, or antigens plus EF plus NEI. (A) Serum anti-PA neutralizing Ab responses. Serum samples were collected one week after the last immunization (Day 21). (A) High affinity PA-specific IgG1 and IgA Ab responses. Antibody titers were assessed by a modified ELISA where low affinity Abs were removed by incubation with urea. (B) PA-specific neutralizing Ab titers were determined by the in vitro macrophage toxicity assay. Thus, NEI promotes high affinity and functionally protective serum antibodies in mice immunized via a non-parenteral route.

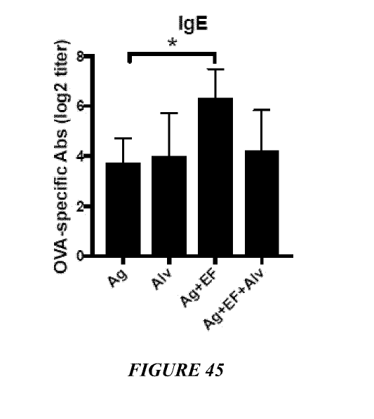

As shown in FIG. 45, sublingual co-application of a NEI suppresses antigen-specific serum IgE responses. Mice were immunized three times at weekly intervals by sublingual application of antigens (OVA plus PA) alone, antigens plus the NEI, antigens plus EF, or antigens plus EF plus NEI. Serum were collected weekly and OVA-specific IgE responses were analyzed by ELISA. Thus, NEI suppresses IgE responses in mice immunized via a non-parenteral route.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

What is claimed is:

1. A method for enhancing an immune response to a vaccine antigen in a subject, comprising co-administering to the subject the vaccine antigen and an adjuvant composition comprising a neutrophil inhibitor in an amount effective to promote an IgG response to the vaccine antigen in the subject.

2. The method of claim 1, wherein the co-administration of the vaccine antigen and the adjuvant composition further promotes an IgA response to the vaccine antigen in the subject.

3. The method of claim 1, wherein the adjuvant composition and vaccine antigen are in the same composition.

4. The method of claim 1, wherein the adjuvant composition and vaccine antigen are administered simultaneously.

5. The method of claim 1, wherein the vaccine antigen is administered prior to the adjuvant composition.

6. The method of claim 1, wherein the vaccine antigen is administered after the adjuvant composition.

7. The method of claim 1, wherein the adjuvant composition is administered sublingually.

8. The method of claim 1, wherein the adjuvant composition is administered intramuscularly, epicutaneously, or subcutaneously.

9. The method of claim 1, wherein the neutrophil inhibitor inhibits neutrophil recruitment.

10. The method of claim 1, wherein the neutrophil inhibitor is a neutrophil elastase inhibitor.

11. The method of claim 1, wherein the neutrophil inhibitor comprises sivelestat, alvelestat, elafin, or a protein of the serpin family.

12. The method of claim 1, wherein the IgG response is an IgG1 response or an IgG2b response.

13. The method of claim 1, wherein the co-administration of the vaccine antigen and the adjuvant composition induces the expression of the mucosal homing receptors CCR9 or α4β7.

14. The method of claim 1, wherein the co-administration of the vaccine antigen and the adjuvant composition induces the expression of BAFF, APRIL, or IL-10.

15. The method of claim 1, wherein the co-administration of the vaccine antigen and the adjuvant composition induces the production of high-affinity IgG antibodies.

16. The method of claim 1, wherein the adjuvant composition further comprises an additional adjuvant.

17. The method of claim 16, wherein the additional adjuvant is an aluminum salt, a TLR9 agonist, a TLR3 agonist, a TLR4 agonist, a TLRS agonist, or a squalene-based oil-in-water emulsion.

18. The method of claim 1, wherein the subject is a mammal.

19. A vaccine composition comprising a vaccine antigen and a neutrophil inhibitor, in an amount effective to promote an IgG response to the vaccine antigen in a subject.

20. An adjuvant composition comprising a neutrophil inhibitor and an additional adjuvant, in an amount effective to promote an IgG response to a vaccine antigen in the subject.
